SEQUENCE LISTING (10 sequences)

SEQ ID NO: 1 - protein, 5 aa, artificial sequences
  SOURCE 1..5: /mol_type=“protein” /note=“CDR-H1” /organism=“artificial sequences”
DTYIH

SEQ ID NO: 2 - protein, 17 aa, artificial sequences
  SOURCE 1..17: /mol_type=“protein” /note=“CDR-H2” /organism=“artificial sequences”
RIYPTNGYTRYADSVKG

SEQ ID NO: 3 - protein, 11 aa, artificial sequences
  SOURCE 1..11: /mol_type=“protein” /note=“CDR-H3” /organism=“artificial sequences”
WGGDGFYAMDY

SEQ ID NO: 4 - protein, 11 aa, artificial sequences
  SOURCE 1..11: /mol_type=“protein” /note=“CDR-L1” /organism=“artificial sequences”
RASQDVNTAVA

SEQ ID NO: 5 - protein, 7 aa, artificial sequences
  SOURCE 1..7: /mol_type=“protein” /note=“CDR-L2” /organism=“artificial sequences”
SASFLYS

SEQ ID NO: 6 - protein, 9 aa, artificial sequences
  SOURCE 1..9: /mol_type=“protein” /note=“CDR-L3” /organism=“artificial sequences”
QQHYTTPPT

SEQ ID NO: 7 - protein, 119 aa, artificial sequences
  SOURCE 1..119: /mol_type=“protein” /note=“heavy chain variable region” /organism=“artificial sequences”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 8 - protein, 108 aa, artificial sequences
  SOURCE 1..108: /mol_type=“protein” /note=“light chain variable region” /organism=“artificial sequences”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 9 - protein, 450 aa, artificial sequences
  SOURCE 1..450: /mol_type=“protein” /note=“heavy chain” /organism=“artificial sequences”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 10 - protein, 214 aa, artificial sequences
  SOURCE 1..214: /mol_type=“protein” /note=“light chain” /organism=“artificial sequences”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2015/0166664 A1
(43) Pub. Date: Jun. 18, 2015

(54) NOVEL THERAPEUTIC TREATMENTS WITH ANTI-HER2 ANTIBODIES HAVING A LOW FUCOSYLATION

(71) Applicant: Glycotope GmbH, Berlin (DE)

(72) Inventors: Steffen Goletz, Berlin (DE); Antje Danielczyk, Berlin (DE)

(73) Assignee: GLYCOTOPE GMBH, Berlin (DE)

(21) Appl. No.: 14/412,797

(22) PCT Filed: Jul. 18, 2013
(86) PCT No.: PCT/EP2013/065189

§ 371 Date: Jan. 5, 2015

Related U.S. Application Data
(60) Provisional application No. 61/673,216, filed on Jul. 18, 2012.
(60) Provisional application No. 61/673,201, filed on Jul. 18, 2012.
(60) Provisional application No. 61/673,229, filed on Jul. 18, 2012.

(30) Foreign Application Priority Data
Dec. 18, 2012 (EP) 12197768.0

(51) Int. Cl. C07K 16/28; A61K 45/06; A61N 5/10; A61K 39/395

(52) U.S. Cl. CPC C07K 16/2863; A61K 39/39533; A61K 45/06; A61N 5/10; C07K 2317/41; C07K 2317/73; A61K 2039/505

(57) ABSTRACT

The present invention pertains to the field of cancer therapy using anti-cancer antibodies. The medical use of anti-HER2 antibodies having improved glycosylation characteristics, in particular a reduced fucosylation, is provided which show enhanced efficacy.

### FIELD OF THE INVENTION

The present invention pertains to novel medical uses of anti-HER2 antibodies having improved glycosylation characteristics. Said anti-HER2 antibodies show therapeutic efficacy where therapy with common therapeutic antibodies and chemotherapeutic agents has failed or is less effective, thereby allowing to successfully treat novel patient groups and in particular patients, that cannot be successfully treated with conventional anti-HER2 antibody therapy. In particular, the present invention provides novel anti-metastatic treatments as well as novel treatments for pretreated patients, including heavily pretreated patients afflicted with a metastazing cancer wherein the cancer or metastases reoccurred despite the prior treatment. Furthermore, the present invention pertains to novel medical uses of anti-HER2 antibodies having improved glycosylation characteristics in the treatment of HER2 positive diseases which show only a low overexpression of HER2, in particular HER2 positive cancers having a HER2 expression of 1+ or 2+ as determined by immunohistochemistry (IHC).

### BACKGROUND OF THE INVENTION

Antibodies are widely used agents in the field of medicine and research. In medicine, they find application in many different fields, in particular as therapeutic agents in the treatment and prophylaxis of a variety of diseases, in particular neoplastic diseases such as cancer. However, therapeutic results obtained by antibody therapy of cancer patients are highly variable. A significant percentage of the therapies using anti-cancer antibodies shows no or only a small alleviation of the disease and sometimes are limited to specific patient groups.

Exemplary established anti-cancer antibodies are antibodies against the human epidermal growth factor receptor 2 (HER2). The human epidermal growth factor receptor 2 (HER2) protein is thought to be a unique and useful target for antibody therapy against cancers that over-express the HER-2/neu gene. HER2 is over-expressed in several cancers, including but not limited to breast cancer, colon cancer, advanced esophageal adenocarcinomas, gastric adenocarcinomas or gastroesophageal junction adenocarcinomas. E.g. HER2 is over-expressed in 20 to 30% of human breast cancers and correlates with a poor clinical prognosis in women with node-positive and node-negative disease. Over-expression of HER2 has also been associated with more aggressive tumors. For treating HER2 positive tumors, such as in particular breast cancer, the use of anti-HER2 antibodies is an established form of therapy. The recombinant humanized anti-HER2 monoclonal antibody trastuzumab (Herceptin®) was approved for clinical use in the United States in 1998 and is approved for the treatment of breast cancer, including metastatic breast cancer and metastatic gastric cancer. Trastuzumab is used as mono- and combination therapy. Trastuzumab is expressed in CHO cells (hamster cells) and therefore is highly fucosylated. Response rates to the antibody given as a single agent (monotherapy) have ranged from 15-26%.

Another anti-HER2 antibody is the antibody pertuzumab (also known as recombinant human monoclonal antibody 2C4; OMNITARG®) which represents the first in a new class of antibodies which are known as HER dimerisation inhibitors (HDI) and functions to inhibit the ability of HER2 to form active heterodimers with other HER receptors (such as EGFR/HER1, HER3 and HER4) and is active irrespective of HER2 expression levels. Pertuzumab blockade of the formation of HER2/HER3 heterodimeres in tumor cells has been demonstrated to inhibit critical cell signalling, which results in reduced tumor proliferation and survival. Pertuzumab has undergone testing as a single agent in the clinic. In a phase I study, patients with incurable, locally advanced, recurrent or metastatic solid tumors that had progressed during or after standard therapy were treated with pertuzumab given intravenously every 3 weeks. Tumor regression was achieved in 3 of 20 patients evaluable for response. 2 Patients had confirmed partial responses. Stable disease lasting for more than 2.5 months was observed in 6 of 21 patients. These results underline the difficulties to achieve even a partial response or stabilization of the disease. Very often, a beneficial effect such as tumor regression or a stabilisation of the disease is only observed for a few months before the disease eventually progresses.

Afucosylated antibodies have been shown to have enhanced antibody-dependent cell-mediated cytotoxicity (ADCC) and therefore provide an opportunity for development of biobetter antibodies. Evidence suggests that the absence of fucose from the primary n-acetylglucosamine, results in increased affinity of binding of IgG1 anitbodies to the FcγRIIIa receptor with consequent increased ADCC efficacy, mediated by natural killer (NK) cells. This is confirmed in studies employing non-fucosylated glycoforms produced in mutant CHO cells that are deficient in addition of the fucose residue, in particular CHO cells in which the α(1-6) fucosyltransferase enzyme has been knocked out. The affinity of the non-fucosylated IgG1 glycoform for FcγRI or the C1 component of complement was reported to be unaffected; a small increase in affinity for FcγRIIa and FcγRIIb was reported, but as the activating/inhibitory ratio was maintained, it was concluded that it would not be functionally significant. The enhanced ADCC observed for afucosylated IgG-Fc results, in part, from the increased affinity overcoming competition of normal serum IgG for FcγRIIIa. Improved ADCC was also presented for afucosylated trastuzumab. The FcγRIIIa receptor is polymorphic and it has been shown that the FcγRIIIa-158V (valine) form has a higher affinity for IgG1 than the FcγRIIIa-158F (phenylalanine) form. It was demonstrated in vitro that fucosylated IgG1 antibody is more efficient at mediating ADCC through homozygous FcγRIIIa-158V bearing cells than through homozygous FcγRIIIa-158F or heterozygous FcγRIIIa-158V/FcγRIIIa-158F cells. It was anticipated, therefore, that similar differences, in ADCC efficacy might pertain in vivo, depending on the polymorphic form of FcγRIIIa expressed. The use of afucosylated antibodies for treating respective subpopulations of weak-responds patients which are F/F homozygous or V/F heterozygousis is suggested in the prior art, for example US 2006/0182741. Afucosylated antibodies and antibodies with a reduced fucose content are also described in EP 1 500 400 and WO 2008/028686. In vitro results for non-focusylated anti-HER2 antibodies are described in Suzuki et al., Clinical Cancer Research 2007; 13:1875-1882; Juntilla et al., Cancer Research 2010; 70:4481-4489 and Zhang et al., mAbs 3:3, 289-298, 2011. In these papers, anti-HER2 antibodies having a reduced fucosylation were compared with the antibody trastuzumab (Herceptin®), which, due to its production in CHO cells has a high fucosylation in the Fc region. The results demonstrate that the anti-HER2 antibody having a reduced fucose content shows superior ADCC activity. However, therapeutic relevance of these results in clinical applications of these antibodies was not yet demonstrated.

A general problem with anti-HER2 antibodies such as trastuzumab is that they are only active on tumors which over-express HER2. Accordingly, only a small population of patients is qualified for a respective treatment. Clinical trastuzumab trials showed that patients with level 0 to 1+ HER2 expression (determined by IHC) regularly do not benefit from the drug and only a few patients with level 2+ expression do benefit from the drug. More patients with level 3+ expression benefit. However, even in the patient groups having a level 3+ there is a substantial amount of no or low responders. It was found that although trastuzumab shows great affinity for the HER2 receptor and a high dose can be administered (due to its low toxicity) at least 70% of the HER2+ patients do not respond to treatment. No or only a reduced anti-tumor activity is even reported for at least 80% of the patients, in particular those of the F/F and F/V receptor allotype. In fact, resistance regularly develops and the disease progresses. In many cases time to progression of the disease is only delayed for a few months if at all.

Patients afflicted with a HER2 positive cancer wherein a treatment with conventional anti-HER2 antibodies such as trastuzumab has failed, respectively wherein the disease progresses despite anti-HER2 antibody treatment, often have limited therapeutic options. This is in particular the case, if the patient has received prior or simultaneous chemotherapeutic treatments which could also not prevent the progression of the disease. Such prior or simultaneous chemotherapeutic treatments are often encountered in the patient group wherein anti-HER2 antibodies such as trastuzumab fail, because trastuzumab is given in metastatic breast cancer as monotherapy for the treatment of patients which have already received at least two chemotherapeutic regimes (and accordingly are pretreated) and in other indications as combination therapy with chemotherapeutic agents such as paclitaxel or docetaxel. If the disease progresses despite multiple treatment with chemotherapeutic agents and/or anti-HER2 antibodies, the survival prognosis of the patient is low. Here, it must also be kept in mind that the general health status of the patient decreases as the number of treatments increase and the disease progresses. Heavily pretreated patients often have a poor performance status (ECOG) and accordingly are excluded from further aggressive treatments such as further chemotherapy. The survival prognosis is particularly low, if the primary cancer metastasizes and continues to metastasize despite treatment.

Metastasis or metastatic disease is the spread of a disease from one organ or part to another non-adjacent organ or part. Cancer occurs after a single cell in a tissue is progressively genetically damaged to produce a cancer stem cell possessing a malignant phenotype. These cancer stem cells are able to undergo uncontrolled abnormal mitosis which serves to increase the total number of cancer cells at that location. When the area of cancer cells at the originating side becomes clinically detectable, it is called a primary tumor. Some cancer cells also acquire the ability to penetrate and infiltrate surrounding normal tissues and the local area, forming a new tumor. The newly formed “daughter” tumor in the adjacent side within the tissue is called a local metastasis. Some cancer cells acquire the ability to penetrate the walls of lymphatic and/or blood vessels, after which they are able to circulate through the bloodstream (circulating tumor cells) to other sides and tissues in the body. This process is known as lymphatic or haematogenous spread. After the tumors cells come to rest at another side, they re-penetrate the vessels or walls and continue to multiply, eventually forming another clinically detectable tumor. This new tumor is known as a metastatic (or secondary) tumor. Metastasis is one hallmark of malignancy. Most tumors in other neoplasms can metastazise, although in varying degrees. When tumor cells metastasize, the new tumor is called a secondary or metastatic tumor, and its cells are similar to those in the original tumor. This means, for example, that, if breast cancer metastasizes to the lungs, the secondary tumor is made up of abnormal breast cells, not of abnormal lung cells.

Metastatic tumors are very common in the late stages of cancer. The spread of metastasis may occur via the blood or the lymphatics or through both routes. If metastasis occurs by lymphatic spread, invasion into the lymphatic system is followed by the transport of tumor cells to regional lmyphnodes and ultimately to other parts of the body. This is the most common route of metastasis for carcinomas. Cancer cells may spread to lymphnodes (regional lymphnodes) near the primary tumor. This is called nodal involvement, positive nodes or regional disease. It is common medical practice to test by biopsy at least to lymphnodes near a tumor site when doing surgery to examine or remove a tumor. Localized spread to regional lymphnodes near the primary tumor is normally not counted as metastasis, although this is a sign of worse prognosis. Transport through lymphatics is the most common pathway for the initial dissemination of carcinomas.

The most common places for the metastasis to occur are the lungs, liver, brain and the bones. However, also skin, metastases are found and occur often in specific cancer types such as breast cancer. Cutaneous metastases (or skin metastases—the terms are used as synonyms herein) refers to the growth of cancer cells in the skin originating from an internal cancer. In most cases, skin metastases develop after the initial diagnosis of the primary internal malignancy (for example breast cancer or lung cancer) and late in the course of the disease. Skin metastasis occurs when cancerous cells brake away from the primary tumor and make their way to the skin through the blood circulation or lymphatic system. Most malignant tumors can produce skin metastasis, but some or more likely to do so than others. The most common sources of skin metastasis in women are the breast (69%), the colon (9%), melanoma (5%), the ovaries (4%) and the lungs (4%). Most skin metastases occur in a body region near the primary tumor. They may break down and ulcerate and accordingly break through the skin. An ulcerating tumor can generally develop in two ways. It may develop as part of a primary tumor or as secondary tumor, i.e. as a metastasis. As described above, if a tumor spreads to the blood and lymphatics system it can travel to the skin and develop into an ulcerating tumor. This is rare and commonly only happens in the advanced stages of cancer. For some people, an ulcerating tumor is the most upsetting aspect of their cancer and it can greatly affect how the patient feels about himself if the ulcerating tumor is visible to other people, for example on the face or abdomen. Furthermore, ulcerating tumors can also smell unpleasant. In breast cancer, the most common sides of skin metastasis are the chest and abdomen. In order to treat skin metastases, the underlying primary tumor needs to be treated. However, in most cases were skin metastasis has occurred the primary cancer is widespread and may be untreatable. In this case, only palliative care can be given.

Treatment and survival of a patient afflicted with metastases is generally determined by whether or not a cancer is local or has spread to other locations. If the cancer spreads to other tissues and organs, it may decrease a patient likelihood of survival. The choice of treatment generally depends on the type of primary cancer, the size and location of the metastases, the patient's age and general health, and the types of treatments used previously. As described above, the mortality rate is particularly high in patients with skin metastases, in particular with advanced ulcerating skin metastases. The appearance of skin metastases signals widespread metastatic disease, resulting in a poor prognosis for the patient.

Clinical oncologists are in agreement that the failure of cancer treatment is not necessarily caused by the growth of the primary tumor, which is generally dealt with using surgery, but rather by the metastatic spread into different organs. Therefore, the effective treatment of metastases, including the prevention of metastases, the inhibition of metastases growth and and the prevention of further spread of metastases is important. It is known that the regression of primary tumors by different anti-cancer drugs is not always indicative for anti-metastatic activity per se. On the contrary, enhanced metastasis has been observed in response to several anti-cancer drugs. Furthermore, chemotherapeutic agents as well as therapeutic antibodies show a different degree of anti-metastatic activity which also depends on the location of the metastasis.

It is known that a therapy with anti-HER2 antibodies can be helpful to treat the primary as well as meastases. However, it is known in the prior art that anti-HER2 antibodies such as trastuzumab show a rather divergent effectiveness on different types of metastases. For example Sawaki et al. (Tumori, 20:40-43, 2004: Efficacy and safety of trastuzumab as a single agent and heavily pre-treated patients with HER-2/NEU-over expressing metastatic breast cancer) analysed how different types of metastases responded to trastuzumab therapy. The patients were confirmed to over-express the HER2 gene product. Sawaki describes as an outcome of the clinical study that 11.5% of the patients had a complete response to the trastuzumab treatment, 11.5% had a partial response, 11.5% had no change and 65% of the patients showed progressive disease. The time to disease progression was 3.1 months (median) and the median duration of the response was 6.4 months. The analysed patients were pre-treated by conventional chemotherapy and were refractory to said therapy. Most of the patients had received multiple chemotherapeutic regiments and therefore, the analysed study population generally had a very poor prognosis. As discussed above, the more unsuccessful treatments a patient receives, the poorer is his prognosis. Sawaki concludes that trastuzumab is active as a single agent in women with HER2 over-expressing metastatic breast cancer that has progressed after chemotherapy even though Sawaki concludes that the effect is considered to lack sufficient efficacy. Furthermore, Sawaki reports that the observed response rates also severely differed by metastatic site. Sawaki concludes that the results obtained in the study suggest that trastuzumab is not effective as a single agent against visceral metastases, in particular liver metastases and lung metastases and brain metastases. A response rate of 50% was seen with skin metastasis, 43% with lymphnodes, 10% with bone metastases but no response regarding lung and liver metastases. However, considering the overall low response rates this underlines that trastuzumab is not effective in a large number of patients and in particular patients afflicted with specific metastases such as lung or liver metastases.

Rossi et al. (Anticancer research 24: 317-320 (2004)) report a case wherein bone marrow metastases occurring in a heavily pretreated patient afflicted with metastatic breast cancer could be effectively treated with trastuzumab. The patient achieved a complete recovery of blood cell counts but died at the end due to a progression of lung metastases. Rossi reports that the median survival time after diagnosis of metastatic breast cancer is 18 to 24 months, but points out that this varies widely according to the metastatic site of the disease. The median survival time has traditionally been lower for patients with visceral disease (6 to 13 months) versus those with only bone disease (18 to 30 months).

Gori et al. (The Oncologist 2007; 12:766-773: Central nervous system metastases in HER-2-positive metastatic breast cancer patients treated with trastuzumab: incidence, survival, and risk factors) describes an observational study to evaluate the incidence of CNS metastases in HER-2-positive metastatic breast cancer patients to define the outcome of patients with CNS metastases and to identify risk factors for relapse. Gori reports that visceral metastases are the dominant site at relapse and that this is associated with a significantly higher risk for CNS metastases. This underlines the importance of an efficient treatment of visceral metastases.

In view of above, it is evident that there is a great demand for improved treatments of HER2 positive neoplastic diseases, in particular metastatic HER2 positive cancers. Furthermore, there is a high demand to provide effective treatment schedules for patients wherein the disease progresses despite previous treatment with anti-HER2 antibodies and/or chemotherapeutic agents, and in particular there is a demand to provide treatment options for heavily pretreated patients. In particular, there is a high demand to provide efficient options for the treatment of HER2 positive metastases, in particular skin metastases, lymphnode metastases and visceral metastases such as lung and liver metastases. Furthermore, there is a demand to provide improved treatments for HER2 positive cancers which only show a low or moderate expression of HER2, in particular HER2 1+ or HER2 2+ as determined by IHC.

### SUMMARY OF THE INVENTION

Anti-HER2 antibodies according to the present invention having a reduced (including absent) fucosylation in their Fc region demonstrate in the clinical trials reported herein remarkable and unexpected therapeutic efficacy for treating a patient with a HER2 positive neoplastic disease, in particular cancer. The anti-HER2 antibodies described herein are therapeutically active against primary cancers and metastases. They were also shown to be therapeutically effective against primary cancers and metastases resistant or refractory to conventional cancer treatments. Furthermore, the anti-HER2 antibodies according to the present invention having a reduced (including absent) fucosylation in their Fc region demonstrate a good therapeutic efficacy against HER2 positive cancers, which show only a low or moderate HER2 expression (e.g. 1+ or 2+ as determined by IHC). In particular, said anti-HER2 antibodies are effective against metastases and primary tumors resistant or refractory to conventional cancer treatments. In particular, the reduced fucose anti-HER2 antibodies according to the present invention showed a high therapeutic efficacy against primary cancers and metastases that were or became resistant to the treatment with conventional anti-HER2 antibodies having a high fucosylation and/or were or became resistant to the treatment with one or more chemotherapeutic agents. Based upon the findings reported herein, the present invention provides novel medical treatment schedules which allow to treat specific patient groups that could not or can no longer be treated with conventional therapy, in particular conventional anti-HER2 antibody therapy, including combination therapies that included conventional anti-HER2 antibodies. In particular, the present invention provides successful treatment schedules for heavily pretreated patients, i.e. patients that have received multiple lines of previous anti-cancer treatments wherein, however, such previous treatments failed and wherein said patients have wide-spread metastases. The data presented in this application demonstrate that such patients can be successfully treated following the teachings of the present invention even if the reduced fucose anti-HER2 antibody according to the present invention is administered as monotherapy. A treatment success was seen with numerous different metastases, including ulcerating skin metastases, lymph node metastases and visceral metastases, in particular lung and liver metastases. The demonstrated strong anti-metastatic efficacy is an important clinical success, as the treatment of this specific patient sub-group of heavily pretreated patients with metastases is particularly difficult and thus, this patient group has a very poor survival prognosis. The present invention provides successful novel treatments for said patients, even in a montherapy setting. Furthermore, as is demonstrated by the data presented herein, a therapeutic success is achieved even when administering low dosages of the anti-HER2 antibody according to the present invention. Furthermore, the therapeutic effect was seen very rapidly, thereby demonstrating the remarkable therapeutic efficacy of the anti-HER2 antibodies according to the present invention in this patient group. E.g. in a heavily pretreated patient afflicted with metastatic breast cancer and large ulcerating skin metastases wherein previous multiple treatments with chemotherapeutic agents and conventional anti-HER2 antibodies failed and the disease progressed, ulcerating skin metastases began to heal already 8 days after the first administration of the anti-HER2 antibody (monotherapy) according to the present invention and could eventually be fully repaired. Furthermore, also in a heavily pretreated patient afflicted with metastatic colon cancer and having lung and liver metastases wherein previous treatments with chemotherapeutic agents and conventional anti-cancer antibodies failed and the disease progressed, a significant reduction (44%) of the target lesions was observed when the reduced fucose anti-HER2 antibody according to the present invention was administered in a monotherapy setting.

Furthermore, the data presented herein also demonstrates that the reduced fucose anti-HER2 antibodies described herein advantageously can be used for treatment of HER2 positive cancers and in particular metastatic cancers which exhibit a low HER2 overexpression of 1+ or 2+ (as determined by immohistochemistry), again also in pretreated patients wherein treatments with numerous other anti-cancer agents failed.

Furthermore, in the performed clinical studies it was shown that the anti-HER2 antibodies according to the present invention are well tolerated and that side effects observed were reduced compared to conventional antibody therapies. This is an important advantage considering the health condition of heavily pretreated patients, which often excludes aggressive therapies such as further chemotherapy.

Furthermore, the data presented in the present application shows a therapeutic effect in patients that could so far not successfully be treated with high fucose anti-HER2 antibodies such as e.g. trastuzumab, e.g. patients afflicted with visceral metastases such as liver and lung metastases.

Based on the above findings, the present invention in a first aspect provides an anti-HER2 antibody having an amount of fucose in the CH2 domain of 50%, preferably 40% or less, 30% or less, 20% or less, preferably 15% or less and most preferred 10% to 0% (reduced fucose anti-HER2 antibody) for treating a human patient with a HER2 positive cancer, wherein the cancer is a metastasizing cancer.

In a second aspect, the present invention provides an anti-HER2 antibody having an amount of fucose in the CH2 domain of 50% or less, preferably 40% or less, preferably 30% or less, 20% or less, more preferred 15% or less, most preferred 10% to 0% (reduced fucose anti-HER2 antibody) for treating a patient with a HER2 positive neoplastic disease, in particular cancer, wherein prior to the treatment with the reduced fucose anti-HER2 antibody said patient has been treated with
a) at least one chemotherapeutic agent;
b) at least one anti-HER2 antibody having an amount of fucose in the CH2 domain of 60% or more, in particular 70% or more (high fucose anti-HER2 antibody), or at least one anti-HER2 antibody which is not glycosylated;
c) optionally radiotherapy; and
d) optionally at least one further therapeutic antibody;
   wherein the preceding treatments a), b), optionally c) and optionally d) occurred in any order sequentially or concurrently. Said cancer may be a metastazing cancer and/or a cancer having a HER2 overexpression of level 2+ or less, such as level 1+, as determined by immunohistochemistry (IHC).

In a third aspect, the present invention provides an anti-HER2 antibody having an amount of fucose in the CH2 domain of 50% or less, preferably 40% or less, 30% or less, 20% or less, preferably 15% or less, most preferred 10% to 0% or 10% to 3% (reduced fucose anti-HER2 antibody) for treating a patient with a HER2 positive neoplastic disease, in particular a HER2 positive cancer, wherein the HER2 positive cancer has a HER2 overexpression of level 2+ or lower, preferably level 1+, as determined by immunohistochemistry (IHC). The reduced fucose anti-HER2 antibody is particularly useful for the treatment of metastasizing cancer. Furthermore, based on the above findings, the present invention provides an anti-HER2 antibody having an amount of fucose in the CH2 domain of 50%, preferably 40% or less, 30% or less, 20% or less, preferably 15% or less, most preferred 10% to 0% or 10% to 3% (reduced fucose anti-HER2 antibody) for treating a human patient with a HER2 positive metastasizing cancer, wherein the HER2 positive cancer has a HER2 overexpression of level 2+ or lower, preferably level 1+, as determined by immunohistochemistry (IHC).

As discussed above, the reduced fucose anti-HER2 antibodies described herein are particularly effective against metastases. Furthermore, they are particularly effective for treatment of pretreated and also heavily pretreated patients and in particular in patients being afflicted with metastases, in particular visceral metastases, lymphnode metastases and ulcerating skin metastases. The therapeutic effects were seen even when administering the reduced fucose anti-HER2 antibody as monotherapy. Therefore, the reduced fucose anti-HER2 antibody according to the present invention proved to be highly effective and novel effective treatment schedules are provided by the present invention. The extent of improvement of the therapeutic efficacy of the lowly fucosylated anti-HER2 antibodies according to the invention and the treatment options resulting thereform were unexpected even in view of existing in vitro data which demonstrate an increased ADCC activity upon defucosylation. In particular the possibility of effectively treating patients being afflicted with metastases that can regularly not be treated with conventional highly fucosylated anti-HER2 antibodies was surprising since this is not simply an improvement of an already existing therapeutic effect but represents a transformation of an ineffective therapeutic agent (the high fucose anti-HER2 antibody) into a highly active therapeutic agent (the reduced fucose anti-HER2 antibody). As described herein, the reduced fucose anti-HER2 antibodies described herein are highly effective even at low dosages and are capable of treating metastases such as ulcerating skin metastases, visceral metastases such as lung metastases and/or liver metastases and lymphnode metastases. Furthermore, the reduced fucose anti-HER2 antibodies were also effective on HER2 positive cancers which only had a low overexpression of HER2 of level 2+ or lower and even level 1+ (as determined by immunohistochemistry). The achieved therapeutic effect is observed very rapidly even when the reduced fucose antibodies are administered as monotherapy. Therefore, the present invention makes an important contribution to existing cancer therapies.

As is apparent from the above and subsequent disclosure, the different aspects of the present invention can be combined. For example, as is shown in the examples, the anti-HER2 antibodies of the invention can be used for treating metastases and primary tumors resistant or refractory to conventional cancer treatments. The possibility to successfully treat such pretreated and also heavily pretreated patients afflicted with metastazing cancer and in particular with existing multiple metastases is an important contribution that is made by the invention. Additionally, it is an advantage that the anti-HER2 antibodies of the invention are effective on HER2 positive cancers having a low overexpression of HER2 of level 2+ or lower. Therefore, treatment options are provided for the aforementioned patients, even if they have such low HER2 expressing cancer.

In a fourth aspect, the present invention is directed to a method of treatment of a patient suffering from a HER2-positive neoplastic disease, comprising administering an anti-HER2 antibody having an amount of fucose in the CH2 domain of 50% or less, preferably 30% or less, more preferably 15% to 0% (reduced fucose anti-HER2 antibody) to said patient in an amount sufficient to treat the neoplastic disease. The features and embodiments of the other aspects of the invention also likewise apply to the method of treatment of the invention. In particular, the HER2-positive neoplastic disease may be a metastasizing cancer as described herein, and/or the patient may have received one or more previous cancer treatments as described herein, and/or the HER2-positive neoplastic disease may be a HER2 positive cancer having a HER2 expression of level 2+ or lower, preferably level 1+, as determined by immunohistochemistry (IHC) as described herein.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody. The antibody is capable of inducing ADCC.

In particular, the antibody may be of any isotype such as IgA, IgD, IgE, IgG or IgM, including any subclass such as IgG1, IgG2, IgG3, IgG4, IgA1 or IgA2. Preferably, the antibody is an IgG antibody, more preferably an IgG1- or IgG2-antibody, in particular an IgG1-antibody. The heavy chain constant regions may be of any type such as γ-, δ-, α-, μ- or ε-type heavy chains. Furthermore, the light chain constant region may also be of any type such as κ- or λ-type light chains. Preferably, the light chain of the antibody is a κ-chain. Preferably the antibody is a full length antibody which in the case of IgG antibodies comprises two full length heavy chains and two full length light chains.

The antigen-binding portion of an antibody usually refers to full length or one or more fragments of an antibody that retain the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and CH1 domains; a F(ab)₂ fragment, a bivalent fragment comprising two Fab fragments, each of which binds to the same antigen, linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR). The “Fab part” of an antibody in particular refers to a part of the antibody comprising the heavy and light chain variable regions (VH and VL) and the first heavy and light chain constant regions (CH1 and CL). In cases where the antibody does not comprise all of these regions, then the term “Fab part” only refers to those of the regions VH, VL, CH1 and CL which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the VH region of the antibody.

The “Fc part” of an antibody in particular refers to a part of the antibody comprising the heavy chain constant regions 2, 3 and—where applicable—4 (CH2, CH3 and CH4). In cases where the antibody does not comprise all of these regions, then the term “Fc part” only refers to those of the regions CH2, CH3 and CH4 which are present in the antibody. Preferably, the Fc part comprises at least the CH2 region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises a Fc receptor binding site or a Fc receptor binding ability. The “Fc part” is capable of inducing ADCC.

For indicating the amino acid positions of the heavy chain and light chain, in particular the variable regions thereof, the Kabat numbering system is used herein (Kabat, E. A. et al. (1991) Sequences of Proteins of Immunological Interest, 5^{th} edition, NIH Publication No. 91-3242). According to said system, the heavy chain variable region comprises amino acid positions from position 0 to position 113 including position 35A, 35B, 52A to 52C, 82A to 82C and 100A to 100K. The CDRs of the heavy chain variable region are located, according to the Kabat numbering, at positions 31 to 35B (CDR1), 50 to 65 (CDR2) and 95 to 102 (CDR3). The remaining amino acid positions form the framework regions FR1 to FR4. The light chain variable region comprises positions 0 to 109 including positions 27A to 27F, 95A to 95F and 106A. The CDRs are located at positions 24 to 34 (CDR1), 50 to 56 (CDR2) and 89 to 97 (CDR3). Depending on the initial formation of the specific gene of an antibody, not all of these positions have to be present in a given heavy chain variable region or light chain variable region. In case an amino acid position in a heavy chain or light chain variable region is mentioned herein, unless otherwise indicated it is referred to the position according to the Kabat numbering.

According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

According to the present invention, the term “humanized antibody” in particular refers to an antibody wherein at least one CDR is derived from a non-human antibody, and wherein the constant regions and at least one framework region of a variable region are derived from a human antibody or a human antibody consensus sequence. Preferably, all CDRs of the heavy chain variable region or, more preferably, all CDRs of the heavy chain variable region and the light chain variable region, are derived from anon-human antibody. Furthermore, preferably all framework regions of the heavy chain variable region or, more preferably, all framework regions of the heavy chain variable region and the light chain variable region, are derived from a human antibody or a human antibody consensus sequence. The CDRs preferably are derived from the same non-human antibody. The first three or all of the framework regions of one variable region preferably are derived from the same human antibody or human antibody consensus sequence, however, the framework regions of the heavy chain variable region do not have to be derived from the same human antibody or human antibody consensus sequence as the framework regions of the light chain variable region. In particular preferred embodiments, the humanized antibody is capable of binding to the same antigens, in particular the same epitopes as the non-human antibody from which the one or more CDRs are derived. Preferably, the CDRs of the humanized antibody which are derived from the non-human antibody are identical to the CDRs of the non-human antibody. Furthermore, the framework regions of the humanized antibody which are derived from the human antibody or human antibody consensus sequence may be identical to the framework regions of the human antibody or human antibody consensus sequence. In another embodiment, the framework regions of the humanized antibody may have one or more amino acid substitutions compared to the framework regions of the human antibody or human antibody consensus sequence from which they are derived. The substituted amino acid residues are preferably replaced by the corresponding amino acid residues of the non-human antibody from which one or more of the CDRs are derived (in particular those corresponding amino acid residues which are at the same position according to the Kabat numbering). In particular, the framework regions of a variable region (heavy chain variable region and/or light chain variable region) of the humanized antibody preferably comprise no more than 30 amino acid substitutions, preferably no more than 25, no more than 20, nor more than 15, no more than 12, no more than 10 or no more than 8 amino acid substitutions. In preferred embodiments, all framework regions of the heavy chain variable region of the humanized antibody, taken together, share a homology or an identity of at least 70%, preferably at least 75%, at least 80%, at least 85% or at least 90%, with the framework regions of the heavy chain variable region of the human antibody or human antibody consensus sequence from which they are derived. Furthermore, all framework regions of the light chain variable region of the humanized antibody, taken together, preferably share a homology or an identity of at least 70%, preferably at least 75%, at least 80%, at least 85% or at least 90%, with the framework regions of the light chain variable region of the human antibody or human antibody consensus sequence from which they are derived. The constant regions of the humanized antibody may be derived from any human antibody or human antibody consensus sequence. In particular, the heavy chain constant regions may be of any type such as γ-, δ-, α-, μ- or ε-type heavy chains. The humanized antibody may thus be of any isotype such as IgA, IgD, IgE, IgG or IgM, including any subclass such as IgG1, IgG2, IgG3, IgG4, IgA1 or IgA2. Preferably, the humanized antibody is an IgG1- or IgG2-antibody, more preferably an IgG1-antibody. Furthermore, the light chain constant region may also be of any type such as κ- or λ-type light chains. Preferably, the light chain of the humanized antibody is a κ-chain. The use of humanized anti-HER2 antibodies as reduced fucose anti-HER2 antibodies is preferred.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g. human germline sequences, or mutated versions of human germline sequences or antibody containing consensus framework sequences derived from human framework sequences analysis, for example, as described in Knappik, et al. (2000. J Mol Biol 296, 57-86). The human antibodies of the invention may include amino acid residues not encoded by human sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. In particular the human antibody can be human monoclonal antibody displaying a single binding specificity which has variable regions in which both the framework and CDR regions are derived from human sequences. Preferably, it is recombinant and is prepared, expressed, created or isolated by recombinant means. Such recombinant human antibodies have variable regions in which the framework and CDR regions are derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Furthermore, the antibody according to the present invention may have been subjected to framework or Fc engineering. Such engineered antibodies include those in which modifications have been made to framework residues within V_{H} and/or V_{L}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies can also be used according to the present invention. In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. E.g., the Fc region can be altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. In one embodiment, the Fc region of the described antibodies is modified to increase the ability of the antibody to mediate antibody dependent cellular cytotoxicity (ADCC) and/or to increase the affinity of the antibody for an Fcγ receptor by modifying one or more amino acids. This approach is described further e.g. in WO00/42072. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604).

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95% or at least 97%. For example, if a framework region of a humanized antibody is derived from or corresponds to a variable region of a particular human antibody, then the amino acid of the framework region of the humanized antibody shares a homology or identity over its entire length with the corresponding framework region of the human antibody of at least 75%, more preferably at least 80%, at least 85%, at least 90%, at least 93%, at least 95% or at least 97%. The “corresponding part” or “corresponding framework region” means that, for example, framework region 1 of a heavy chain variable region (FRH1) of a target antibody corresponds to framework region 1 of the heavy chain variable region of the reference antibody. The same is true, for example, for FRH2, FRH3, FRH4, FRL1, FRL2, FRL3 and FRL4. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically.

The term “trastuzumab” as used herein in particular refers to the antibody trastuzumab having the amino acid sequences of the trastuzumab antibody as used in the medicament Herceptin® (Roche). As long as the circumstances do not indicate otherwise, the antibody trastuzumab also has in its Fc part the or a similar high fucose glycosylation pattern as the trastuzumab antibody used in the medicament Herceptin (Roche), wherein the fucosylation is at least 60%, in particular at least 70%. Circumstances that indicate a different glycosylation pattern are, for example, the reference to “Fuc− trastuzumab”. The term Fuc− trastuzumab in particular refers to an antibody binding the same epitope as trastuzumab and having amino acid sequences which are at least 85%, preferably at least 90%, more preferred at least 95% identical to those of the trastuzumab antibody as used in the medicament Herceptin® (Roche), wherein, however, the Fuc− trastuzumab has a lower amount of fucose in its Fc part than the trastuzumab antibody used in the medicament Herceptin® and in particular has a fucosylation in the Fc part of 50% or less, 30% or less, preferably 20% or less, more preferred 15% or less and most preferred 10% to 0%.

The term “HER2” or “HER2/neu” according to the present invention in particular refers to the human epidermal growth factor receptor 2, also known as ErbB-2 or CD340. HER2 is a receptor tyrosine kinase comprising an extracellular ligand binding domain, a membrane-spanning domain and an intracellular kinase domain. Upon binding of its ligand, the HER2 forms homodimers or heterodimers with other ErbB receptors and its kinase function is activated, resulting in the autophosphorylation of several tyrosines of the intracellular domain. An anti-HER2 antibody is an antibody which is capable of specifically binding HER2. Furthermore, an anti-HER2 antibody is generally capable of inhibiting the proliferation of HER2 positive human cancer cells.

The term “antibody”, in particular “anti-HER2 antibody”, as used herein especially refers to a population of antibodies or a composition comprising antibodies, in particular a population of anti-HER2 antibodies or a composition comprising anti-HER2 antibodies suitable for pharmaceutical administration. All or substantially all of the antibodies in the population of antibodies or the composition comprising the antibodies in particular have the same amino acid sequence. A glycosylation feature of an antibody such as an anti-HER2 antibody in particular refers to the average glycosylation of the antibodies in the population or composition. For example, according to the invention the (percentage) amount of fucose in the Fc part and thus the CH2 domain of an antibody in particular refers to the percentage of all carbohydrate chains attached to the corresponding glycosylation site in the CH2 domain of the antibodies in the antibody population or antibody composition which comprise a fucose residue. Said carbohydrate chains include the carbohydrate chains attached to the glycosylation site corresponding to amino acid position 297 according to the Kabat numbering of the heavy chain of IgG-type antibodies (for example amino acid position 301 in SEQ ID NO: 9). The N-linked glycosylation at Asn297 is conserved in mammalian IgGs as well as in homologous regions of other antibody isotypes. Preferably, only fucose residues are considered which are bound via an α1,6-linkage to the GlcNAc residue at the reducing end of the carbohydrate chain. If the amount of fucose in the CH2 domain of a specific antibody species (e.g. anti-HER2 antibodies) is mentioned, then only the carbohydrate chains attached to the CH2 domain and thus the Fc part of the antibody molecules of said specific antibody species in an antibody population or composition are considered for determining the percentage amount of fucose. Carbohydrate chains in the Fab part of the antibody, if present, are not considered. Likewise, the (percentage) amount of bisecting N-acetylglucosamine (bisGlcNAc) of an antibody in particular refers to the percentage of all carbohydrate chains attached to the Fc part of the antibodies in the antibody population which comprise a bisGlcNAc residue. bisGlcNAc refers to a GlcNAc residue attached to the central mannose residue in complex type N-glycans. Furthermore, the (percentage) amount of galactose of an antibody in a composition in particular refers to the percentage of all carbohydrate chains attached to the Fc part of the antibodies in the antibody population which comprise at least one galactose residue.

According to the invention, the term “glycosylation site” in particular refers to an amino acid sequence which can specifically be recognized and glycosylated by a natural glycosylation enzyme, in particular a glycosyltransferase, preferably a naturally occurring mammalian or human glycosyltransferase. In particular, the term “glycosylation site” refers to an N-glycosylation site, comprising an asparagine residue to which the carbohydrate is or will be bound. In particular, the glycosylation site is an N-glycosylation site which has the amino acid sequence Asn-Xaa-Ser/Thr/Cys, wherein Xaa is any amino acid residue. Preferably, Xaa is not Pro.

The term “conjugate” particularly means two or more compounds which are linked together so that at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “patient” in particular refers to a human being.

The term “HER2 positive cancer” according to the invention which can be treated with the reduced fucose anti-HER2 antibodies described herein in particular refers to a primary cancer or tumor which expresses HER2/neu. HER2 positive cancers include but are not limited to breast cancer, gastric cancer, carcinomas, colon cancer, transitional cell carcinoma, bladder cancer, urothelial tumors, uterine cancer, advanced esophageal adenocarcinomas, gastric adenocarcinomas or gastroesophageal junction adenocarcinomas, ovarian cancer, lung cancer, lung adenocarcinoma, bronchial cancer, endometrial cancer, kidney cancer, pancreatic cancer, thyroid cancer, colorectal cancer, prostate cancer, cancer of the brain, cervical cancer, intestinal cancer, liver cancer, saliviary gland cancer and malignant rhabdoid tumor and in particular metastatic forms of the foregoing. Preferably, the HER2 positive cancer to be treated with the reduced fucose anti-HER2 antibody is selected from breast cancer, colon cancer and bladder cancer, in particular metastatic breast cancer and metastatic colon cancer. Most preferably, the HER2 positive cancer is breast cancer, in particular metastatic breast cancer. Preferably, the HER2 positive cancer overexpresses HER2 and/or shows HER2 gene amplification. Accordingly, a HER2 positive cancer in particular is a cancer which comprises tumor cells and/or metastatic cells which overexpress HER2. Preferably at least 5%, more preferably at least 10%, at least 25% or at least 50% of the cancer cells overexpress HER2 and/or show HER2 gene amplification. A HER2 positive cancer in particular refers to a cancer which has a HER2 overexpression of at least level 1+ (HER2 1+), preferably at least level 2+ (HER2 2+), more preferably level 3+(HER2 3+), as determined by immunohistochemistry. In certain embodiments the HER2 positive cancer is a cancer which has a HER2 expression of level 2+ or lower, preferably level 1+ or lower as determined by immunohistochemistry. As is shown by the examples, the reduced fucose anti-HER2 antibodies described herein are therapeutically effective on respective cancers showing only a moderate to low HER2 overexpression. Immunohistochemistry in this respect refers to the immunohistochemical staining of fixed tumor samples and the analysis of the staining. A HER2 expression level of 0 (HER2 0) refers to no staining or a membrane staining in less than 10% of the tumor cells, in particular less than 20,000 HER2 per cell. HER2 1+ refers to a weak membrane staining in more than 10% of the tumor cells, wherein the cell membranes are only partially stained, in particular about 100,000 HER2 per cell. HER2 2+ refers to a weak to moderate staining of the entire membrane in more than 10% of the tumor cells, in particular about 500,000 HER2 per cell. HER2 3+ refers to a strong complete membrane staining in more than 10% of the tumor cells, in particular about 2,000,000 HER2 per cell. The HER2 expression preferably is determined using histological samples comprising cancer cells, in particular formalin-fixed, paraffin-embedded cancer tissue samples. The immunohistochemical assay used for determining the HER2 overexpression preferably includes (i) contacting the sample comprising the cancer cells with a primary antibody against HER2, followed by (ii) contacting the sample with a secondary antibody which is directed against the primary antibody and is coupled to a visualization agent such as an enzyme which catalyzes a reaction having an visible end product, for example horseradish peroxidase. Suitable HER2 immunohistochemistry kits are HercepTest (Dako Denmark A/S) and Pathway HER2 (Ventana Medical Systems, Inc.). HER2 positive neoplastic diseases also include cancers which are positive for HER2 gene amplification as determined by fluorescence in situ hybridization (FISH) or chromogene in situ hybridization (CISH). A cancer is positive for HER2 gene multiplication according to the FISH assay if the number of copies of the HER2 gene in the tumor cells is at least 2-times the number of copies of chromosome 17 or if the tumor cells comprise at least 4 copies of the HER2 gene. A cancer is positive for HER2 gene multiplication according to the CISH assay if at least 5 copies of the HER2 gene per cell nucleus are present in at least 50% of the tumor cells.

By “metastasis” or “metastases” is meant the spread of cancer cells from its original site to another part of the body. As described above in the background of the invention, the formation of metastasis is a very complex process and normally involves detachment of cancer cells from a primary tumor, entering the body circulation and settling down to grow within normal tissues elsewhere in the body. For details, it is referred to the respective disclosure which also applies here. As described herein, the HER2 positive cancer that is treated with the fucose reduced anti-HER2 antibody is according to the preferred embodiment a metastatic cancer, also referred to herein as metastasizing cancer. The metastases can be distant metastases. The metastases are in particular HER2 positive as described above for the HER2 positive cancer; it is referred to the above disclosure which also applies here. Specific types of metastases that can be successfully treated with the reduced fucose anti-HER2 antibody as described herein are skin metastases, lymphnode metastases and visceral metastases. “Skin metastasis” or “skin metastases”, the terms are used as synonyms, refers to the growth of cancer cells in the skin originating from an internal cancer. The development and characteristics of skin metastases were described in detail in the background of the invention, it is referred to the respective disclosure which also applies here. In particular, the skin metastases can be ulcerating skin metastases. “Visceral metastasis” or “visceral metastases” in particular refers to metastases in the viscera, the internal organs of the body, specifically those within the chest such as heart or lungs or the abdomen, such as the liver, pancreas or intestines. In particular, the term “visceral metastasis” refers to metastases in the lung and/or the liver.

The term “failed treatment” or “treatment failure” or related terms according to the invention particularly refer to treatments of cancer which result in progression of the disease. Progression of disease in particular refers to (i) the further growth of an existing tumor, in particular by at least 25%; (ii) the growth or the formation of one or more new metastases of an existing type; (iii) the formation of one or more further metastases of a different type; (iii) the formation of further lesions and/or (iv) the increase of the size of one or more lesions. The further growth of a tumor in particular refers to an increase in tumor volume by at least 25%. The increase of the size of a lesion in particular refers to an increase in lesion size by at least 25%.

The term “successful treatment” or “treatment success” or related terms according to the invention particularly refer to treatments of HER2 positive cancer or metastases which result in a stabilization of the disease, a partial remission and/or a full remission of the disease. A successful treatment preferably includes one or more of the following (i) the inhibition of tumor growth; (ii) the reduction of tumor size; (iii) the prevention of further metastases of the same type and/or of a different type; (iv) the reduction of the number of metastases; (v) the prevention of further lesions; (vi) the reduction of the number of lesions; (vii) the reduction of the size of one or more lesions; and/or (viii) the reduction of pain. The reduction of tumor size in particular refers to a decrease in tumor volume by at least 25%, including a remission wherein the tumor volume is reduced by 25 to 50%, a partial remission wherein the tumor volume is reduced by more than 50%, and a complete remission wherein the tumor volume is reduced by 100%. The reduction of the size of a lesion in particular refers to a decrease in the lesion size by at least 25%, including a reduction wherein the lesion size is reduced by 25 to 50%, a partial reduction wherein the lesion size is reduced by more than 50%, and a complete reduction wherein the lesion size is reduced by 100%. A lesion in particular refers to a lesion caused by a primary tumor and/or by one or more metastases. A particular example of a lesion is a skin ulcer, in particular caused by a skin metastasis. A successful treatment in particular also includes treatments which result in an increase in progression-free survival and/or an increase in lifespan, in particular a progression-free survival or a remaining lifespan of at least 1 month, of at least 2 months, preferably at least 3 months, at least 4 months, at least 6 months, at least 9 months or at least 1 year, even more preferably of at least 1.5 years, at least 2 years, at least 3 years, at least 4 years or at least 5 years. A “stable disease” and accordingly a stabilization of the disease in particular includes (i) a variation in the tumor and/or metastases volume by less than 25% and (ii) no change in the number of metastases. The successful treatment preferably is determined for an observation period of at least 1 month, more preferably at least 2 months, at least 3 months, at least 4 months, at least 6 months, at least 9 months or at least 1 year, even more preferably at least 1.5 years, at least 2 years, at least 3 years, at least 4 years or at least 5 years.

Treatment failure as well as a successful treatment is established based on the medical judgement of a practitioner ascertained by the results from clinical and laboratory data that are generally known in the art to assess patient treatment. Such data may be obtained by way of example, from clinical examination, cytological and histological techniques, endoscopy and laparoscopy, ultrasound, CT, PET and MRI scans, chest x-ray and mammography. Furthermore, RECIST criteria may be used to determine the tumor response.

The term “surgery” according to the invention in particular refers to a surgical removal (resection or ectomy) of tissue comprising all or a part of a tumor, in particular a primary tumor such as a breast tumor, and/or one or more metastases.

An “adjuvant therapy” in particular refers to the treatment of cancer after surgery.

A “neoadjuvant therapy” in particular refers to the treatment of cancer prior to surgery.

A “palliative therapy” in particular refers to a cancer therapy that is given specifically to address symptom management without expecting to significantly reduce the cancer. Palliative care is directed to improving symptoms associated with incurable cancer. The primary objective of palliative care is to improve the quality of the remainder of a patient's life. Pain is one of the common symptoms associated with cancer. Approximately 75% of terminal cancer patients have pain. Pain is a subjective symptom and thus it cannot be measured using technological approaches. The majority of cancer patients experience pain as a result of tumor mass that compresses neighboring nerves, bone, or soft tissues, or from direct nerve injury (neuropathic pain). Pain can occur from affected nerves in the ribs, muscles, and internal structures such as the abdomen (cramping type pain associated with obstruction). Many patients also experience various types of pain as a direct result of follow-up tests, treatments (surgery, radiation, and chemo-therapy) and diagnostic procedures (i.e., biopsy). A therapeutically useful palliative therapy is able to reduce pain.

The term “radiotherapy”, also known as radiation therapy, particularly means the medical use of ionizing radiation to control or kill malignant cells. Radiotherapy may be used in combination with surgery, as adjuvant and/or neoadjuvant therapy, or without surgery, for example to prevent tumor recurrence after surgery or to remove a primary tumor or a metastasis.

The term “pharmaceutical composition” and similar terms particularly refers to a composition suitable for administering to a human, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The terms “antibody composition” and “composition comprising an antibody” are used interchangeably herein. The antibody composition may be a fluid or solid composition, and also includes lyophilized or reconstituted antibody compositions. Preferably a fluid composition is used, more preferably an aqueous composition. It preferably further comprises a solvent such as water, a buffer for adjusting and maintaining the pH value, and optionally further agents for stabilizing the antibody or preventing degradation of the antibody. The antibody composition preferably comprises a reasonable amount of antibodies, in particular at least 1 fmol, preferably at least 1 pmol, at least 1 nmol or at least 1 μmol of the antibody. A composition comprising a specific antibody may additionally comprise further antibodies. However, preferably, a composition comprising a specific antibody does not comprise other antibodies apart from the specific antibody. In particular, at least 75%, preferably at least 80%, at least 85%, at least 90%, at least 95%, at least 97%, at least 98% or at least 99%, most preferably about 100% of the antibodies in an antibody composition are directed to or bind to the same antigen or epitope. Accordingly, the antibody as used herein preferably refers to an antibody that is substantially free of other antibodies having different antigenic specificities. The antibody composition preferably is a pharmaceutical composition.

### DETAILED DESCRIPTION OF THE INVENTION

The remarkable therapeutic results achieved with the teachings of the present invention and the novel treatment options provided were briefly described in the summary of the present invention to which it is referred. Based on the data shown in the examples, the present invention provides different novel treatment options for treating HER2 positive neoplastic diseases, in particular HER2 positive cancer, which may also be combined.

In a first aspect, the invention provides an anti-HER2 antibody having an amount of fucose in the CH2 domain of 50% or less, 40% or less, 30% or less, 20% or less, preferably 15% or less or 10% to 0% (reduced fucose anti-HER2 antibody) for treating a patient with a metastasizing HER2 positive neoplastic disease, in particular metastasizing cancer. Thus, the present invention provides an anti-HER2 antibody having an amount of fucose in the CH2 domain of 50%, preferably 40% or less, 30% or less, 20% or less, preferably 15% or less and most preferred 10% to 0% (reduced fucose anti-HER2 antibody) for treating a human patient with a HER2 positive cancer, wherein the cancer is a metastasizing cancer.

Furthermore, based on the data shown in the examples, the present invention provides in a second aspect an anti-HER2 antibody having an amount of fucose in the CH2 domain of 50% or less, 40% or less, 30% or less, 20% or less, preferably 15% or less or 10% to 0% (reduced fucose anti-HER2 antibody) for treating a patient with a HER2 positive neoplastic disease, in particular cancer, wherein prior to the treatment with the reduced fucose anti-HER2 antibody said patient has been treated with
a) at least one chemotherapeutic agent;
b) at least one anti-HER2 antibody having an amount of fucose in the CH2 domain of 60%, in particular 70% or more (high fucose anti-HER2 antibody), or at least one anti-HER2 antibody which is not glycosylated;
c) optionally radiotherapy; and
d) optionally at least one further therapeutic antibody;
   wherein the preceding treatments a), b), c) and d) occurred in any order sequentially or concurrently. The advantages of said novel therapeutic teaching were described above and are also described subsequently.

In a third aspect, based on the data shown in the examples, the present invention provides an anti-HER2 antibody having an amount of fucose in the CH2 domain of 50% or less, 40% or less, 30% or less, 20% or less, preferably 15% or less, 10% to 0% or 10% to 3% (reduced fucose anti-HER2 antibody) for treating a patient with a HER2 positive neoplastic disease, in particular metastasizing cancer, wherein the HER2 positive neoplastic disease has a HER2 overexpression of level 2+ or lower, preferably level 1+, as determined by immunohistochemistry (IHC). In particular, the present invention provides an anti-HER2 antibody having an amount of fucose in the CH2 domain of 50%, preferably 40% or less, 30% or less, 20% or less, preferably 15% or less and most preferred 10% to 0% or 10% to 3% (reduced fucose anti-HER2 antibody) for treating a human patient with a HER2 positive cancer, wherein the cancer is a metastasizing cancer which has a HER2 overexpression of level 2+ or lower, preferably level 1+, as determined by immunohistochemistry (IHC).

Furthermore, treatments are disclosed wherein prior to the treatment with the reduced fucose anti-HER2 antibody said patient has been treated with
a) at least one chemotherapeutic agent and/or
b) at least one anti-HER2 antibody having an amount of fucose in the CH2 domain of 60% or more, preferably 70% or more (high fucose anti-HER2 antibody), or at least one anti-HER2 antibody which is not glycosylated;
c) optionally radiotherapy; and
d) optionally at least one further therapeutic antibody;
   wherein said preceding treatments occurred in any order sequentially or concurrently. Preferred embodiments of the individual treatments are described subsequently and in the claims to which it is referred. As is shown in the examples, the reduced fucose anti-HER2 antibodies described herein demonstrate a high therapeutic efficacy in particular against numerous metastases and furthermore, allow the successful treatment of pretreated patients and also heavily pretreated patients, wherein the cancer progressed despite the received previous anti-cancer treatments. Furthermore, the different aspects of the invention can be combined. For example, the reduced fucose anti-HER2 antibodies show an improved anti-metastatic activity and can advantageously be used for the treatment of metastases which failed prior antibody and/or chemotherapeutic treatment. In particular, the reduced fucose anti-HER2 antibodies can be used for treatment of skin metastases such as ulcerating skin metastases and visceral metastases such as lung and/or liver metastases as well as metastases of the lymph nodes. Furthermore, the therapeutic effect was seen with HER2 positive cancers which showed a low HER2 overexpression as determined by IHC (1+ and 2+) and accordingly, the reduced fucose anti-HER2 antibody can be used for treating a patient being afflicted with a HER2 positive cancer showing a HER2 overexpression of only 1+ or 2+. Furthermore, the therapeutic efficacy was seen in a monotherapy setting and also at low dosages. Therefore, the present invention provides important novel treatment options as are also described in further detail above.

### Reduced Fucose Anti-HER2 Antibody According to the Invention

The reduced fucose anti-HER2 antibodies described herein have an unexpectedly high therapeutic efficacy and allow the treatment of patients and sub-group of patients that can not or could not be treated with conventional therapy. Even metastases and tumors which are resistant to treatment with established anti-cancer agents such as high fucose anti-HER2 antibodies and/or chemotherapeutic agents can be successfully treated with the reduced fucose anti-HER2 antibody according to the invention. Furthermore, the reduced fucose anti-HER2 antibodies described herein are therapeutically effective against HER2 positive cancers which show only a low or moderate HER2 expression (e.g. or level 1+ or 2+ as determined by IHC). A therapeutic effect is seen even if the reduced fucose anti-HER2 antibodies are administered as monotherapy and even administered at low dosages.

An important feature of the reduced fucose anti-HER2 antibody according to the invention is the improved glycosylation pattern in the Fc part of the antibody. The reduced fucose anti-HER2 antibody preferably is an IgG antibody, more preferably an IgG1 antibody, which has a glycosylation site in the second constant domain of the heavy chain (CH2). This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to the optional additional amino acids which may be present in the variable region, this conserved glycosylation site is at amino acid position 301 of SEQ ID NO: 9. In particular, in at least 80%, preferably at least 85%, at least 90% or at least 95%, more preferably in at least 98% of the reduced fucose anti-HER2 antibody comprised in a composition, the glycosylation site of at least one CH2 domain, preferably of both CH2 domains, carries a carbohydrate structure. The amount of fucosylation as described herein is determined at this glycosylation site in the Fc region. In certain embodiments, the reduced fucose anti-HER2 antibody does not comprise further glycosylation sites and/or does not carry carbohydrate structures in any of the variable domains, the CH1 domain and the CL domain.

The reduced fucose anti-HER2 antibody has an amount of fucose at the glycosylation site in the CH2 domain which is 50% or less, 40% or less, 30% or less or even 20% or less, more preferably 15% or less, most preferably 10% or less or is afucosylated and thus does not comprise any fucose. In particular embodiments, the reduced fucose anti-HER2 antibody comprises at least a residual amount of fucose of at least 2%, at least 3% and preferably at least 5%. The term “amount of fucose” in particular refers to the relative amount of carbohydrate chains carrying a fucose unit of all carbohydrate chains attached to the reduced fucose anti-HER2 antibodies in a composition comprising the reduced fucose anti-HER2 antibodies.

Anti-HER2 antibodies having a reduced amount of fucosylation, including antibodies which do not carry any fucose, as used herein can be obtained by various means. E.g. the anti-HER2 antibody can be expressed in a host cell with altered glycosylation machinery. Cells with altered glycosylation machinery have been described in the art and can be used as host cells to produce recombinant anti-HER2 antibodies having a reduced fucosylation in their Fc region as described herein. For example, EP 1,176,195 by Hang et al. describes a cell line with a functionally disrupted FUT8 gene, which encodes a fucosyl transferase, such that antibodies expressed in such a cell line exhibit hypofucosylation. Therefore, in one embodiment, the antibodies comprised in the compositions of the invention are produced by recombinant expression in a cell line which exhibits hypofucosylation pattern, for example, a mammalian cell line with deficient expression of the FUT8 gene encoding fucosyltransferase. WO03/035835 describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). The antibodies comprised in the compositions of the invention can be produced in a yeast or a filamentous fungi engineered for mammalian-like glycosylation pattern, and capable of producing antibodies lacking fucose as glycosylation pattern (see for example EP1297172B1).

Preferably, the reduced fucose anti-HER2 antibody is obtained by recombinant expression in a human cell line which has a reduced or even no fucosylation capacity. A respective reduced or absent fucosylation capacity can be achieved e.g. by reducing the expression of enzymes necessary for fucosylation (e.g. FUT8 or GMD), or by eliminating the respective gene functions, e.g. by gene knockout. The reduced fucose anti-HER2 antibody preferably is produced recombinantly in a human cell line, preferably a human blood cell line, in particular in a human myeloid leukemia cell line. The cell line used for producing the reduced fucose anti-HER2 antibody preferably has a reduced or absent fucosylation activity and/or the reduced fucose anti-HER2 antibody is produced under conditions which result in a reduced or even absent fucosylation of the antibody. As described herein, a reduced or absent fucosylation activity can be achieved by manipulating the expression or activity of enzymes necessary for fucosylation (e.g. FUT8 or GMD). Preferred human cell lines which can be used for production of the reduced fucose anti-HER2 antibody, in particular Fuc− trastuzumab, as well as suitable production procedures are described in WO 2008/028686 A2, herein incorporated by reference.

Furthermore, the level of fucosylation of the reduced fucose anti-HER2 antibody may be reduced after their production by the cell line, for example by in vitro treatment with a fucosidase.

As discussed above, the reduced fucose anti-HER2 antibody preferably has a glycosylation profile and is obtained by expression in a human cell line, preferably a human a human myeloid leukemia cell line. A human glycosylation profile is preferably characterized in that at least 70%, preferably at least 80%, at least 85% or more preferred by at least 90% of the carbohydrate chains attached to the Fc part of the reduced fucose anti-HER2 antibody are complex type glycan structures, preferably biantennary complex type glycan structures. The reduced fucose anti-HER2 antibody having a human glycosylation profile particularly does not comprise detectable amounts of N-glycolyl neuraminic acid (NeuGc) and/or Galα1,3-Gal structures. Respective glycosylation structures are found in antibodies that are produced in non-human cell lines such as rodent cell lines. Furthermore, it preferably comprises detectable amounts of α1,6-coupled N-acetyl neuraminic acid (NeuAc).

In preferred embodiments, the reduced fucose anti-HER2 antibody comprises an amount of bisecting N-acetylglucosamine (bisGlcNAc) which is higher than the amount of bisGlcNAc of the high fucose anti-HER2 antibody. It may comprise an amount of bisGlcNAc in the carbohydrate chain attached to the CH2 domain of at least 2%, preferably at least 5%, at least 8% or more preferred at least 10%. The amount of bisGlcNAc preferably is in the range of from 5% to 50%, preferably from 7% to 40%, more preferably from 8% to 25% and more preferred from 10% to 25%. The term “amount of bisGlcNAc” in particular refers to the relative amount of carbohydrate chains carrying a bisecting N-acetylglucosamine unit of all carbohydrate chains attached to the reduced fucose anti-HER2 antibodies in a composition comprising the reduced fucose anti-HER2 antibodies. It was found that reducing the amount of core fucose and at the same time increasing the amount of bisGlcNAc in the Fc glycosylation provides a reduced fucose anti-HER2 antibody which shows a strong increase in tumor lysis, a strong anti-metastatic efficacy and furthermore, allows to efficiently treat a broader patient spectra.

Furthermore, the reduced fucose anti-HER2 antibody preferably comprises an amount of galactose of at least 50%, preferably at least 55%, at least 60% or at least 65%. The amount of galactose preferably is in the range of from 50% to 95%, more preferably from 55% to 90%, most preferably from 60% to 80%. The term “amount of galactose” in particular refers to the relative amount of galactosylated carbohydrate chains, that is carbohydrate chains comprising at least one galactose unit, of all carbohydrate chains attached to the reduced fucose anti-HER2 antibodies in a composition comprising the reduced fucose anti-HER2 antibodies. In certain embodiments, the reduced fucose anti-HER2 antibody comprises a relative amount of carbohydrate chains carrying two galactose units of at least 10%, preferably at least 15%, more preferably at least 18% or at least 20%. The relative amount of carbohydrate chains carrying two galactose units in particular is in the range of from 10% to 50%, preferably from 15% to 40%, more preferably from 18% to 30%.

The amount of bisGlcNAc and/or the amount of galactose preferably refer only to the amount of bisGlcNAc and the amount of galactose, respectively, in the carbohydrate chains attached to the CH2 domain of the reduced fucose anti-HER2 antibody and thus in the Fc part of the antibody. A glycosylation comprising bisGlcNAc and galactose as described above is also characteristic for a human glycosylation pattern and can be obtained by expressing the anti-HER2 antibodies in a human cell line as described above.

The reduced fucose anti-HER2 antibody preferably is an IgG antibody, more preferably an IgG1 antibody. It has the ability of specifically binding its target epitope and the ability of binding to Fcγ receptors, in particular to the Fcγ receptor IIIa. The reduced fucose anti-HER2 antibody is capable of inducing an antibody-dependent cellular cytotoxicity (ADCC) reaction. The reduced fucose anti-HER2 antibody is capable of inducing a stronger ADCC than the high fucose anti-HER2 antibody. In particular, the reduced fucose anti-HER2 antibody is at least 2-fold, at least 3-fold, at least 5-fold, at least 7-fold, at least 10-fold, at least 20-fold, at least 30-fold, at least 40-fold or at least 50-fold more potent in inducing ADCC than the high fucose anti-HER2 antibody, as determined in in vitro ADCC assays, in particular in ADCC assays as described in Example 15, below. As is shown therein, an up to 10-140 fold improvement of ADCC anti-tumor activity was observed when comparing the Fuc− trastuzumab (invention) with the Fuc+ antibody (prior art). The higher potency in inducing ADCC preferably refers to the X-fold lower concentration of the reduced fucose anti-HER2 antibody necessary for inducing the same level of ADCC (such as ratio of lysed target cells), preferably the same specific lysis at 95% of maximal lysis of the high fucose anti-HER2 antibody, compared to the high fucose anti-HER2 antibody. For example, if the reduced fucose anti-HER2 antibody induces the same level of ADCC at a 5-fold lower concentration than the high fucose anti-HER2 antibody, then the reduced fucose anti-HER2 antibody is 5-fold more potent in inducing ADCC than the high fucose anti-HER2 antibody. As is shown by the examples, a 10 to 140fold less antibody concentration was needed for the same ADCC response when using the reduced fucose anti-HER2 antibody compared to a corresponding high fucose anti-HER2 antibody. Alternatively, the higher potency in inducing ADCC may refer to the X-fold higher ADCC level (such as ratio of lysed target cells) induced by the reduced fucose anti-HER2 antibody at the same concentration, preferably 10 ng/ml, as the high fucose anti-HER2 antibody. For example, if the reduced fucose anti-HER2 antibody induces a 5-fold higher level of ADCC than the high fucose anti-HER2 antibody at the same antibody concentration, then the reduced fucose anti-HER2 antibody is 5-fold more potent in inducing ADCC than the high fucose anti-HER2 antibody. The X-fold higher potency in inducing ADCC may in particular refer to ADCC induced with effector cells of donors having the FcγRIIIa-158F/F allotype, or with effector cells of donors having the FcγRIIIa-158V/V allotype, or with effector cells of donors having the FcγRIIIa-158F/V allotype. Preferably, the X-fold higher potency in inducing ADCC is determined as an average of the ADCC induced for each of the different FcγRIIIa allotypes. As is shown by the examples, a reduced fucose antibody according to the present invention shows compared to a corresponding high fucose anti-HER2 antibody generally a higher ADCC, an effect which is even more prominent on cancer cells being characterized by a low HER2 overexpression. Therefore, the anti-HER2 antibody can effectively mediate ADCC at all ADCC receptor allotypes and furthermore, this effect was seen with lower HER2 expressing tumors (1+ as determined by IHC).

The reduced fucose anti-HER2 antibody comprises a heavy chain variable region (VH) and a CH2 domain, more preferably the domains VH, CH1, CH2 and CH3. Furthermore, the reduced fucose anti-HER2 antibody preferably comprises a light chain variable region (VL), preferably the domains VL and VH. The reduced fucose anti-HER2 antibody may comprise two heavy chains and two light chains. It preferably is a recombinant monoclonal antibody such as a human, humanized or chimeric antibody and preferably is a humanized antibody.

The reduced fucose anti-HER2 antibody mediates ADCC and is according to a preferred embodiment capable of specifically binding to the extracellular part of HER2/neu, in particular to domain IV of HER2/neu and has at least one, preferably at least two, more preferably all of the following activities: (i) it is capable of blocking ligand binding to HER2, (ii) it is capable of blocking activation of HER2/neu, in particular of the kinase activity of HER2/neu and/or (iii) it is capable of reducing the amount of HER2/neu at the cell surface, in particular by inducing internalization of HER2/neu into the cell. Preferably, the reduced fucose anti-HER2 antibody has all of the aforementioned characteristics. Preferably, the reduced fucose anti-HER2 antibody shows cross-specificity with the antibody trastuzumab and in particular binds to the same epitope as the antibody trastuzumab. Preferably, the reduced fucose anti-HER2 antibody is equivalent to trastuzumab in binding and Fv mediated anti-tumor properties, however, shows increased ADCC mediated anti-tumor properties due to the improved glycosylation. In preferred embodiments, the reduced fucose anti-HER2 antibody comprises the same heavy chain and preferably also light chain CDR sequences as trastuzumab. In particular, the entire amino acid sequence of the heavy chain and preferably also of the light chain of the reduced fucose anti-HER2 antibody are at least 85% identical, at least 90% identical, at least 95% identical or at least 97% identical to the corresponding amino acid sequences of trastuzumab. Preferably, the amino acid sequences of the reduced fucose anti-HER2 antibody are derived from the corresponding amino acid sequences of trastuzumab.

In certain embodiments the reduced fucose anti-HER2 antibody comprises a heavy chain variable region comprising the complementarity determining regions (CDRs) CDR-H1, CDR-H2 and CDR-H3, wherein the CDR-H1 has the amino acid sequence of SEQ ID NO: 1 and/or CDR-H2 has the amino acid sequence of SEQ ID NO: 2 and/or CDR-H3 has the amino acid sequence of SEQ ID NO: 3. Preferably, the heavy chain variable region of the reduced fucose anti-HER2 antibody comprises all three of these CDR sequences and in particular comprises the amino acid sequence of SEQ ID NO: 7. In preferred embodiments, the reduced fucose anti-HER2 antibody comprises a light chain variable region comprising the complementarity determining regions (CDRs) CDR-L1, CDR-L2 and CDR-L3, wherein the CDR-L1 has the amino acid sequence of SEQ ID NO: 4 and/or CDR-L2 has the amino acid sequence of SEQ ID NO: 5 and/or CDR-L3 has the amino acid sequence of SEQ ID NO: 6. Preferably, the light chain variable region of the reduced fucose anti-HER2 antibody comprises all three of these CDR sequences and in particular comprises the amino acid sequence of SEQ ID NO: 8. Furthermore, in certain embodiments the reduced fucose anti-HER2 antibody comprises a heavy chain variable region which comprises an amino acid sequence which is at least 85% identical, at least 90% identical or at least 95% identical to the amino acid sequences of SEQ ID NO: 7, and/or a light chain variable region which comprises an amino acid sequence which is at least 85% identical, at least 90% identical or at least 95% identical to the amino acid sequences of SEQ ID NO: 8. Preferably, the heavy chain(s) of the reduced fucose anti-HER2 antibody comprises the amino acid sequence of SEQ ID NO: 9 or an amino acid sequence which is at least 85% identical, at least 90% identical or at least 95% identical thereto. Furthermore, the light chain(s) of the reduced fucose anti-HER2 antibody preferably comprises the amino acid sequence of SEQ ID NO: 10 or an amino acid sequence which is at least at least 85% identical, at least 90% identical or at least 95% identical thereto. As described above, the reduced fucose anti-HER2 antibody preferably is equivalent to trastuzumab in binding and Fv mediated anti-tumor properties.

In certain preferred embodiments, the reduced fucose anti-HER2 antibody comprises a heavy chain variable region comprising the amino acid sequence of SEQ ID NO: 7 or an amino acid sequence which is at least 80%, preferably at least 90% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3. Preferably, the reduced fucose anti-HER2 antibody additionally comprises a light chain variable region comprising the amino acid sequence of SEQ ID NO: 8 or an amino acid sequence which is at least 80%, preferably at least 90% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6.

According to one embodiment, the reduced anti-HER2 antibody mediates ADCC and is capable of specifically binding to HER2 and blocking dimerization of HER2/neu, in particular heterodimerization of HER2/neu with other members of the epidermal growth factor receptor family such as HER1, HER3 and HER4. Preferably, the reduced fucose anti-HER2 antibody shows cross-specificity with the antibody pertuzumab and in particular binds to the same epitope as the antibody pertuzumab. In preferred embodiments, the reduced fucose anti-HER2 antibody comprises the same heavy chain and preferably also light chain CDR sequences as pertuzumab. In particular, the entire amino acid sequence of the heavy chain and preferably also of the light chain of the reduced fucose anti-HER2 antibody are at least 80% identical, preferably at least 90% identical, at least 95% identical or at least 97% identical to the corresponding amino acid sequences of pertuzumab. Preferably, the amino acid sequences of the reduced fucose anti-HER2 antibody are derived from the corresponding amino acid sequences of pertuzumab and the reduced fucose anti-HER2 antibody is equivalent to pertuzumab in binding and Fv mediated anti-tumor properties, however, shows increased ADCC mediated anti-tumor properties due to the improved glycosylation described herein.

In one embodiment, the reduced fucose anti-HER2 antibody is a conjugate comprising the antibody conjugated to a further agent such as a therapeutically active substance. The further agent preferably is useful in therapy and/or monitoring of cancer. For example, the further agent may be selected from the group consisting of radionuclides, chemtotherapeutic agents, antibodies, in particular those of different species and/or different specificity than the reduced fucose anti-HER2 antibody, enzymes, interaction domains, detectable labels, toxins, cytolytic components, immunomodulators, immunoeffectors, MHC class I or class II antigens, radioisotopes and liposomes. The further agent, if comprised, may be covalently, in particular by fusion or chemical coupling, or non-covalently attached to the antibody. A particular preferred further agent is a radionuclide or a cytotoxic agent capable of killing cancer cells, such as a chemotherapeutic agent, in particular those described herein elsewhere. Specific examples of chemotherapeutic agents that can be conjugated as further agent include alkylating agents such as cisplatin, anti-metabolites, plant alkaloids and terpenoids, vinca alkaloids, podophyllotoxin, taxanes such as taxol, topoisomerase inhibitors such as irinotecan and topotecan, or antineoplastics such as doxorubicin. The reduced fucose anti-HER2 antibody may be conjugated to any of the chemotherapeutic agents and/or antibodies described herein. According to one embodiment, the reduced fucose anti-HER2 antibody is not conjugated to a further agent which is a therapeutically active substance. According to one embodiment, which was also used in the examples, the reduced fucose anti-HER2 antibody is not conjugated to a further agent.

The Treatment with the Reduced Fucose Anti-HER2 Antibody

As demonstrated in the clinical data shown in the examples, the reduced fucose anti-HER2 antibody according to the present invention inter alia shows a high anti-metastatic activity and thus allows the treatment of metastases that can or could not be treated with a corresponding high fucose anti-HER2 antibody. The reduced fucose anti HER2 antibodies show in the patient groups specifically defined herein unexpectedly high therapeutic efficacy even when used as single therapeutic agent. Besides successful treatment of cancer including metastazing cancer, the anti-HER2 antibodies of the invention allow the treatment of HER2 positive cancers having a HER2 expression of level 1+ or 2+ (as determined by IHC) and/or the treatment of pretreated patients as described herein, including heavily pretreated patients. In particular, a prominent effect was seen in the treatment of metastases, such as in particular in the treatment of ulcerating skin metastases, lymph node metastases and visceral metastases such as lung and liver metastases. These effects were also seen in heavily pretreated patients, wherein preceding treatments with anti-cancer agents such as chemotherapeutic agents and/or antibody therapies, in particular with anti-HER2 antibodies, failed. Thus, the reduced fucose anti-HER2 antibody can be used for treatment as monotherapy, even in heavily pretreated patients. Using the reduced fucose anti-HER2 antibody as monotherapy has the advantage that a therapeutic effect can be achieved while only minor side effects can be expected. This is an advantage when treating patients with advanced metastatic cancer, wherein the disease progressed besides preceding treatments with multiple lines of chemotherapy and/or antibody therapy, as this patient group often is in a poor health conditions and thus, is excluded from further aggressive treatment.

However, the reduced fucose anti-HER2 antibody according to the present invention can also be used in combination therapy wherein the cancer is additionally treated with one or more anti-cancer therapeutic agents such as chemotherapeutic agents or further anti-cancer antibodies to further improve the therapeutic benefit for the patient. As the reduced fucose anti-HER2 antibody according to the present invention is effective at low dosages and in particular in lower dosages than conventional high fucose anti-HER2 antibodies, such combination therapies provide again novel and useful therapeutic options, in particular for heavily pretreated patients. In certain embodiments, the reduced fucose anti-HER2 antibody is used in combination with one or more anti-cancer agents such as chemotherapeutic agents and/or one or more further antibodies which are different from the reduced fucose anti-HER2 antibody. Here, combination therapies can be used that are established for high fucose anti-HER2 antibodies, in particular trastuzumab. The treatment can also be combined with radiotherapy.

Anti-cancer agents that can be used in combination with the reduced fucose anti-HER2 antibody may be selected from any chemotherapeutic agent, in particular chemotherapeutic agents known to be effective for treatment of HER2 positive cancers. Particularly, preferred are combinations with anti-cancer agents that are used for trastuzumab (Herceptin®). The combination partner maybe selected from the group consisting of taxanes such as paclitaxel (Taxol), docetaxel (Taxotere) and SB-T-1214; cyclophosphamide; lapatinib; capecitabine; cytarabine; vinorelbine; bevacizumab; gemcitabine; maytansine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues, selective estrogen receptor modulators and estrogen receptor downregulators. If used as combination therapy, the reduced fucose anti-HER2 antibody is preferably used in combination with a taxane such as paclitaxel (Taxol), docetaxel (Taxotere). This particularly, if the reduced fucose anti-HER2 antibody corresponds to trastuzumab, e.g. has the same CDR sequences, in particular the same overall sequences as trastuzumab. Here, basically the same combination schedules and administration schemes can be used as are used in the prior art when using a high fucose anti-HER2 antibody, e.g. trastuzumab, in combination therapy. Suitable combinations of the reduced fucose anti-HER2 antibody based on established trastuzumab therapies include but are not limited to combination therapies with:
(i) as part of a treatment regimen comprising doxorubicin, cyclophosphamide and either paclitaxel or docetaxel;
(ii) docetaxel and carboplatin;
(iii) paclitaxel;
(iv) cisplatin, capecetabine or 5-fluorouracil.

The reduced fucose anti-HER2 antibody can be used subsequent to anthracycline therapy.

Furthermore, therapeutic antibodies can be used as combination partner for the reduced fucose anti-HER2 antibody. It may be any antibody that is useful in cancer therapy which is different from the reduced fucose anti-HER2 antibody. In particular, the further antibody is approved for cancer treatment by an administration such as the U.S. Food and Drug Administration (FDA), the European Medicines Agency (EMA, formerly EMEA) and the Bundesinstitut für Arzneimittel and Medizinprodukte (BfArM). Examples of the further antibody that can be used for combination treatment with the reduced fucose anti-HER2 antibody are anti-HER2 antibodies such as pertuzumab (which is particularly feasible if the reduced fucose anti-HER2 antibody shows cross-specificity with trastuzumab and preferably is a reduced fucose trastuzumab antibody), anti-EGFR antibodies such as cetuximab (Erbitux), panitumomab (Vectibix) and nimotuzumab (Theraloc); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).

The data presented herein demonstrates that the treatment with the reduced fucose anti-HER2 antibody is successful and/or is more efficient than a treatment with a high fucose anti-HER2 antibody, using comparable dosage regiments. As is shown by the data presented herein, the treatment with the reduced fucose anti-HER2 antibody succeeds while the treatment with another antibody, in particular with a high fucose anti-HER2 antibody such as trastuzumab, failed. This treatment success is seen with primary cancers as well as in the treatment of metastases and cancer having a HER2 expression of level 2+ or 1+(as determined by IHC). This effect is seen when using comparable dosage regiments or even when using lower dosages.

The data presented in this application shows that the treatment with the reduced fucose anti-HER2 antibody is successful for patients being homozygous for valine in amino acid position 158 of the Fcγ receptor IIIa (FcγRIIIa-158V/V) and may also be more efficient for said patients than a treatment with a corresponding high fucose anti-HER2 antibody. Furthermore, the presented data shows that the treatment with the reduced fucose anti-HER2 antibody is successful for patients being homozygous for phenylalanine in amino acid position 158 of the Fcγ receptor IIIa (FcγRIIIa-158F/F) and patients being heterozygous for valine and phenylalanine in amino acid position 158 of the Fcγ receptor IIIa (FcγRIIIa-158V/F) and is more efficient for said patients than a treatment with the corresponding high fucose anti-HER2 antibody. Furthermore, the data shows that the treatment with the reduced fucose anti-HER2 antibody can be successfully used for treatment of patients of every Fcγ receptor IIIa allotype in particular all F allotypes (F/F and F/V) and/or is more efficient for said patients than a treatment with the corresponding high fucose anti-HER2 antibody. Thereby, the present invention makes a significant contribution in providing an improved therapy for patients, in particular heavily pretreated patients, afflicted with metastatic cancer as the treatment according to the present invention is available to all members of said group of patients which generally has low survival chance and limited treatment options.

Furthermore, the reduced fucose anti-HER2 antibody taught herein shows enhanced ADCC response not only on cancer cells showing a strong HER2 overexpression (e.g. 3+ as determined by IHC—see e.g. the examples made with SK-BR-3 having approx. 1*10⁶ molecules per cell) but also on cancer cells showing a lower HER2 expression (e.g. 1+ or 2+ as determined by IHC—see e.g. the examples made with MCF-7 cells having approx. 3.5*10⁴ molecules/cell). Therefore, more patients will benefit from the novel treatments described herein and in particular patients being afflicted with a HER2 positive cancer, including metastazing cacer, which is 1+ or 2+ and in particular having a F/F or F/V allotype.

The reduced fucose anti-HER2 antibody provided herein preferably is for treatment of a HER2 positive primary tumor, a HER2 positive recurrent tumor and/or HER2 positive metastases of such tumors, and in particular is useful for treatment before, during or after surgery and for the prevention or treatment of metastases. As is demonstrated by the present invention, the treatments with the reduced fucose anti-HER2 antibody described herein are particularly useful for the treatment, including prevention, of metastases such as skin metastases, in particular ulcerating skin metastases, lymph node metastases, and visceral metastases such as lung metastases and liver metastases. As is shown by the data provided herein the treatment with the reduced fucose anti-HER2 antibody as described herein can be successfully used for the treatment of lesions caused by a HER2 positive tumor or metastasis, in particular for the treatment of skin lesions such as skin ulcerations or lymph node lesions. Furthermore, it was observed that the treatment with the reduced fucose anti-HER2 antibody described herein can be used for the treatment of pain and thus, can be used as palliative therapy for patients with incurable cancer.

The reduced fucose anti-HER2 antibody in particular is for the treatment of a patient as adjuvant therapy. In certain embodiments, the reduced fucose anti-HER2 antibody is for the treatment of a patient as neoadjuvant therapy or in a combined neoadjuvant-adjuvant therapy. Furthermore, the reduced fucose anti-HER2 antibody is for the treatment of a patient as palliative therapy.

As is shown by the examples, the treatment with the reduced fucose anti-HER2 antibody as taught herein is therapeutically successful and in particular can result in tumor or metastases remission or a stabilization of the disease. In particular, in the analyzed heavily pretreated patients at least stabilizations of the disease and partial responses were observed, what are important successes in the patient group of heavily pretreated patients which basically have no or only limited therapeutic options. In particular, the examples show that the treatment with the reduced fucose anti-HER2 antibody described herein may result in the inhibition of tumor growth, the reduction of tumor size; the prevention of further metastases (either of the same or of a different type) and/or the reduction of the number or size of metastases. In particular, an impressive reduction of lesions caused by the primary tumor and/or metastases was observed, in particular with respect to skin metastasis, including ulcerating skin metastasis, lymph node metastases and visceral metastases, including lung and liver metastases. A reduction of mediastinal adenopathies was also observed. Due to the therapeutic effects obtained with the treatment of the present invention, progression-free survival and/or an increase in lifespan of the patients can be achieved.

As is shown by the examples, the reduced fucose anti-HER2 antibody taught herein is highly effective and therefore, a therapeutic response is seen rapidly. This is an important advantage in the patient group being afflicted with metastasizing cancer, as well as in the patient group of heavily pretreated patients, wherein multiple prior treatments have failed. As is shown by the examples, the reduced fucose anti-HER2 antibody taught herein can also be used for treating the patient group being afflicted with metastasizing cancer, wherein multiple prior treatments have failed. A therapeutic effect of the treatment with the reduced fucose anti-HER2 antibody, in particular at least a partial response, is preferably obtained at least after the second administration of the reduced fucose anti-HER2 antibody, preferably already after the first administration of the reduced fucose anti-HER2 antibody. As is shown by the examples, a considerably reduction of ulcerating skin metastases was observed after the first treatment with the reduced fucose anti-HER2 antibody according to the present invention. In certain embodiments, a therapeutic effect is obtained after 8 weeks or less, preferably 7 weeks or less, 6 weeks or less, 5 week or less, 4 weeks or less, 3 weeks or less or 2 weeks or less, more preferably 1 week or less after the first administration of the reduced fucose anti-HER2 antibody.

### The Preceding Treatments

The present inventors found that the reduced fucose anti-HER2 antibody according to the present invention shows high therapeutic efficacy and clinical success even in patients which failed multiple prior anti-cancer treatments, in particular pretreatments with chemotherapeutic agents and/or other anti-cancer antibodies, in particular high fucose anti-HER2 antibodies. The observed effects are remarkable as a cancer therapy is more prone to failure the further the disease has progressed and in particular if metastasis has progressed. After multiple treatments, the cancer cells are highly mutated and thereby more easily evade treatment. Furthermore, the tumor load, i.e. the number of tumor cells in the patient, increases with progression of the disease. At higher tumor cell numbers, the killing of some tumor cells may be outweighed by the proliferation of the remaining tumor cells. The same applies to the development of metastases. Hence, the shown therapeutic effects of the reduced fucose anti-HER2 antibody in heavily pretreated patients and in particular in patients with wide spread metastases is impressive and unexpected and also provide novel treatment options for novel patient groups.

In view of these findings, the reduced fucose anti-HER2 antibody according to the invention is suitable for treatment of a HER2 positive neoplastic disease, in particular HER2 positive cancer in a patient who has received one or more previous treatments of said HER2 positive neoplastic disease. According to one embodiment, said HER2 positive neoplastic disease, in particular said HER2 positive cancer is metastasizing. The preceding treatments of the neoplastic disease include treatments with one or more chemotherapeutic agents, radiation treatments (radiotherapy), treatments with one or more therapeutic antibodies which are different from the reduced fucose anti-HER2 antibody, in particular treatments with one or more high fucose antibodies which preferably correspond regarding their efficacy to the reduced fucose anti-HER2 antibody (e.g. the reduced fucose anti-HER2 antibody is equivalent to trastuzumab in binding and Fv mediated anti-tumor properties) and combinations of two or more of these treatments. In particular, at least one pretreatment with a high fucose anti-HER2 antibody such as trastuzumab occurred either as monotherapy or combination therapy.

Furthermore, the HER2 positive neoplastic disease may have been treated by surgery prior to the treatment with the reduced fucose anti-HER2 antibody. In particular, the preceding treatment of the patient involved cancer surgery, preferably a surgical removal of at least a part of the primary tumor and/or of metastases.

In preferred embodiments, the patient was subject to two or more, preferably three or more, more preferably four or more, five or more, six or more, seven or more or eight or more preceding anti-cancer treatments prior to the treatment with the reduced fucose anti-HER2 antibody. The preceding treatments preferably comprise at least one treatment with a high fucose anti-HER2 antibody such as in particular trastuzumab either as monotherapy or in combination with a further therapy such as one or more chemotherapeutic agents and/or radiotherapy and/or one or more further antibodies which are directed against an antigen different from HER2. The preceding treatment with the high fucose anti-HER2 antibody may have also involved the use of a high fucose anti-HER2 antibody such as trastuzumab, which is conjugated to a further agent, such as in particular a chemotherapeutic agent such as maytansine. In one embodiment, the high fucose anti-HER2 antibody that was used the pretreatment was not conjugated to a further agent. According to one embodiment, an aglycosylated anti-HER2 antibody was used in the preceding treatment.

In particular embodiments, the patient has been treated with at least two, preferably at least three, at least four, at least five, at least six, at least seven, at least eight, at least nine or at least ten different anti-cancer agents such as chemotherapeutic agents and/or therapeutic antibodies prior to the treatment with the reduced fucose anti-HER2 antibody described herein.

One or more, in particular all of the preceding treatments have failed and the HER2 positive cancer reoccurred or progressed following the preceding treatments.

### The High Fucose Anti-HER2 Antibody Used in the Preceding Treatment

In preferred aspects and embodiments of the invention, the reduced fucose anti-HER2 antibody is used after the failed treatment of the patient with a high fucose anti-HER2 antibody. Details regarding the high fucose anti-HER2 antibody and particular embodiments thereof were already described above. Preferably, the reduced fucose anti-HER2 antibody and the high fucose anti-HER2 are based on the same antibody and thus in particular bind the same antigen and comprise the same CDR regions but differ from each other in their glycosylation in the Fc region, in particular in their amount of fucose. The reduced fucose anti-HER2 antibody has a lower amount of fucose than the high fucose anti-HER2 antibody and is capable of mediating a stronger ADCC response. Furthermore, it preferably has a higher amount of bisGlcNAc as described above.

The high fucose anti-HER2 antibody preferably has an amount of fucose in its CH2 domain which is 65% or more, 70% or more, or 75% or more. Respective high fucose antibodies are obtained when producing the antibody in standard cell lines such as CHO cells or SP2/0 cells. E.g. the antibody trastuzumab (Herceptin®) which is produced in CHO cells is a high fucose anti-HER2 antibody with more than 70% fucose in the carbohydrate chain that is attached to the CH2 domain. In preferred embodiments, the amount of fucose in the CH2 domain of the reduced fucose anti-HER2 antibody is at least 20 percentage points, preferably at least 30 percent points, more preferably at least 40 percentage points, at least 50 percentage points or at least 60 percentage points, or even at least 70 percentage points lower than the amount of fucose in the CH2 domain of the high fucose anti-HER2 antibody. E.g. if the high fucose anti-HER2 antibody has a fucose content of 70% and the reduced fucose anti-HER2 antibody has a fucose content that is 60 percentage points lower, it has a fucose content of 10%. According to one embodiment, the reduced fucose anti-HER2 antibody is afucosylated and does not comprise fucose.

In further embodiments, the high fucose anti-HER2 antibody that was used in the previous treatment of the patient has an amount of bisGlcNAc in the CH2 domain of 10% or less, 7% or less or 5% or less, more preferably 3% or less or does not comprise bisGlcNAc. The amount of bisGlcNAc of the reduced fucose anti-HER2 antibody preferably is at least 5 percentage points, more preferably at least 7 percentage points, most preferably at least 10 percentage points higher than the amount of bisGlcNAc of the high fucose anti-HER2 antibody. Furthermore, the high fucose anti-HER2 antibody may comprise an amount of galactose in the CH2 domain of 70% or less, 60% or less or 55% or less, in particular 50% or less. The amount of galactose of the reduced fucose anti-HER2 antibody preferably is at least 10 percentage points higher, more preferably at least 15 percentage points higher or at least 20 percentage points higher, most preferably at least 25 percentage points higher than the amount of galactose of the high fucose anti-HER2 antibody.

The high fucose anti-HER2 antibody preferably is of the same antibody type as the reduced fucose anti-HER2 antibody, and in particular is an IgG antibody, preferably an IgG1 antibody. Preferably, the high fucose anti-HER2 antibody is capable of specifically binding to the same epitope as the reduced fucose anti-HER2 antibody and/or shows cross-specificity with the reduced fucose anti-HER2 antibody. In certain embodiments, the high fucose anti-HER2 antibody has heavy chain and/or light chain amino acid sequences which are at least 80%, at least 90% or at least 95%, more preferably 100% identical to the corresponding amino acid sequences of the reduced fucose anti-HER2 antibody. In particular, the amino acid sequences of the heavy chain CDRs and/or the light chain CDRs are identical to the corresponding amino acid sequences of the CDRs of the reduced fucose anti-HER2 antibody. In preferred embodiments, the high fucose anti-HER2 antibody that was used in the pretreatment is the antibody trastuzumab (Herceptin) or shows cross-specificity with the antibody trastuzumab.

According to one embodiment, the high fucose anti-HER2 antibody that was used in the pretreatment is capable of blocking ligand binding and/or dimerization of HER2/neu, in particular heterodimerization of HER2/neu with other members of the epidermal growth factor receptor family such as HER1, HER3 and HER4. In certain embodiments, the high fucose anti-HER2 antibody is the antibody pertuzumab (Omnitarg) or shows cross-specificity with the antibody pertuzumab.

According to one embodiment, the high fucose anti-HER2 antibody that was used in the pretreatment specifically binds to an epitope of HER2 which is different from the epitope of the reduced fucose anti-HER2 antibody. In this embodiment, the reduced fucose anti-HER2 antibody and the high fucose anti-HER2 antibody have different CDR sequences. According to one embodiment, they have at least one difference in their mode of action.

The high fucose anti-HER2 antibody may be a complete antibody or a fragment or derivative of an antibody. As described above one embodiment, the high fucose anti-HER2 antibody used in the pretreatment may be conjugated to a further therapeutic agent. Examples of suitable therapeutic agent are radionuclides and chemotherapeutic agents, in particular chemotherapeutic agents as described herein, for example maytansine. According to one embodiment, the high fucose anti-HER2 antibody is no conjugate. The preceding treatment with the high fucose anti-HER2 antibody may be a monotherapy or a combination therapy together with one or more chemotherapeutic agents and/or one or more further antibodies and/or radiotherapy. Suitable chemotherapeutic agents and further antibodies are those described herein elsewhere.

As is shown in the examples, the reduced fucose anti-HER2 antibody used according to the present invention has a higher therapeutic efficacy than the high fucose anti-HER2 antibody that was used in the preceding treatment and furthermore in effective therapeutic settings, wherein the high fucose antibody did not show any effects. Details of the therapeutic effects and the treatable patient groups are described elsewhere herein. It was also observed that the therapeutic efficacy of the reduced fucose anti-HER2 antibody is still higher than that of a corresponding high fucose anti-HER2 antibody even when the reduced fucose anti-HER2 antibody is administered at the same dose but less frequently than the high fucose anti-HER2 antibody and/or when the reduced fucose anti-HER2 antibody is administered at the same frequency but at a lower dose than the high fucose anti-HER2 antibody. Therefore, advantageously, the dosages can be lowered and treatment cycles can be prolonged when using the reduced fucose anti-HER2 antibody according to the invention.

In further embodiments, the reduced fucose anti-HER2 antibody is used after the failed treatment of the patient with an anti-HER2 antibody which is not glycosylated. An antibody which does not have any glycosylation at the Fc part shows a reduced binding to Fc receptors and hence, is not capable of mediating a strong ADCC activity. The features and embodiments described herein with respect to the high fucose anti-HER2 antibody likewise apply to the non-glycosylated anti-HER2 antibody. In particular, the non-glycosylated anti-HER2 antibodies encompass antibody fragments which do not comprise a CH2 domain, especially antibody fragments which do not comprise an Fc region.

### The Antibodies Directed Against Other Antigens Used in the Preceding Treatment

Further therapeutic antibodies which are different from the reduced fucose anti-HER2 antibody also include antibodies which are directed against other antigens and/or do not specifically bind HER2. These further antibodies that could have been used in the pretreatment preferably specifically bind antigens which are present on tumor cells and which preferably are not present on non-tumor cells or are present on non-tumor cells in a lower amount or at sites which are not accessible for the antibodies. Preferably, the further antibodies are approved for cancer treatment by an administration such as the U.S. Food and Drug Administration (FDA), the European Medicines Agency (EMA, formerly EMEA) and the Bundesinstitut für Arzneimittel and Medizinprodukte (BfArM). Preferred examples of the further antibody are anti-EGFR antibodies such as cetuximab (Erbitux), panitumomab (Vectibix) and nimotuzumab (Theraloc); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).

The further antibody may be a complete antibody or a fragment or derivative of an antibody. In one embodiment, the further antibody is conjugated to a further therapeutic agent. Examples of such therapeutic agents are radionuclides and chemotherapeutic agents, in particular chemotherapeutic agents as described herein. According to one embodiment, the further antibody that was used in the preceding treatment is no conjugate.

### The Chemotherapeutic Agents Used in the Preceding Treatment

In certain embodiments, the preceding treatments include one or more treatments with a chemotherapeutic agent or with a combination of two or more chemotherapeutic agents, optionally in combination with one or more therapeutic antibodies different from the reduced fucose anti-HER2 antibody. The chemotherapeutic agents may be any chemotherapeutic agents and may be selected from the group consisting of cyclophosphamide; lapatinib; capecitabine; cytarabine; vinorelbine; bevacizumab; gemcitabine; maytansine; anthracyclines such as daunorubicin, doxorubicin, epirubicin, idarubicin, valrubicin and mitoxantrone; taxanes such as paclitaxel (Taxol), docetaxel (Taxotere) and SB-T-1214; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin); and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues. In particular, the preceding treatment included one or more treatments with a taxane.

### Preceding Treatment Schedules

Exemplary preceding treatments the patient received after which the reduced fucose anti-HER2 antibody is used to treat the HER2 positive cancer are given in the following and the pretreatment involves at least one, preferably at least two or at least three of the following treatments:
at least one treatment with trastuzumab (Herceptin®) as monotherapy and/or trastuzumab (Herceptin) in combination with a chemotherapeutic agent, in particular in combination with a taxane such as docetaxel and vinorelbine and/or
at least one trastuzumab monotherapy and at least one, preferably at least two combination therapies involving trastuzumab; and/or
at least one treatment with at least one taxane, preferably at least two separate treatments with one, two or more different taxanes, in particular with paclitaxel and docetaxel as monotherapy or combination therapy; and/or
at least one treatment with a platinum based chemotherapeutic agent such as cisplatin, preferably in combination with a chemotherapeutic agent such as gemcitabine; and/or
at least one radiotherapy, preferably as adjuvant therapy; and/or
at least one, preferably at least two, at least three or at least four treatments with a chemotherapeutic agent or a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine; and/or
at least one, preferably at least two or at least three treatments with a combination of different chemotherapeutic agents such as a combination of folinic acid and fluorouracil and oxaliplatin (FOLFOX), a combination of folinic acid and fluorouracil and irinotecan (FOLFIRI), and a combination of tegafur-uracil and calcium folinate; and/or
at least one, preferably at least two or at least three treatments with a therapeutic antibody which is different from the reduced fucose anti-HER2 antibody, in particular with an anti-EGFR antibody such as panitumomab or cetuximab, and/or with an anti-VEGF antibody such as bevacizumab, optionally in combination with one or more chemotherapeutic agents.

The above preceding treatments could have been used as adjuvant and/or neoadjuvant therapies and the preceding treatments of the cancer may here and preferably include surgery. In preferred embodiments, the reduced fucose anti-HER2 antibody is for treating cancer in a patient after one or more, preferably two or more, three or more, four or more, five or more, six or more, seven or more or eight or more of the above treatments in any order. In particular, the preceding treatment has involved the use of a high fucose anti-HER2 antibody such as trastuzumab (Herceptin®).

### The HER2 Positive Neoplastic Disease and the Patient to be Treated

The HER2 positive neoplastic disease which is to be treated by the reduced fucose anti-HER2 antibody preferably is a HER2 positive cancer as described in detail above. Therein, also preferred types of HER2 positive cancers were described and it is referred to the above disclosure to avoid repetitions. As described therein, the HER2 positive cancer can in particular be selected from the group consisting of breast cancer, colorectal cancer, colon cancer, bladder cancer, ovarian cancer, gastric cancer, esophagus cancer, lung cancer such as non-small cell lung carcinoma (NSCLC), bronchial cancer and salivary gland cancer such as parotid gland carcinoma. In certain embodiments, which are preferred, the cancer is a metastasizing cancer. The HER2 positive cancer may include any type of metastases, such as skin metastases, lymph node metastases, visceral metastases such as lung metastases, liver metastases and/or brain metastases. In certain embodiments, the cancer is a cancer having a HER2 expression of level 2+ or 1+ as determined by IHC and optionally may be metastasizing cancer having these characteristics. The advantages and specific treatment schedules that become possible due to the present invention were described above, it is referred to the above disclosure.

In certain embodiments, the reduced fucose anti-HER2 antibody is for the treatment of metastasizing cancer wherein the primary cancer or tumor already developed one or more metastases. The one or more metastases in particular are present in tissue which is different from the tissue from which the primary cancer or tumor developed.

In preferred embodiments, the patient to be treated is afflicted with HER2 positive breast cancer, in particular metastasizing breast cancer. Breast cancer includes ductal carcimona in situ, invasive ductal carcinoma, lobular carcinoma in situ, invasive lobular carcinoma, medullary carcinoma, Paget's disease of the nipple and metastatic breast cancer. A specific example of such HER2 positive breast cancer is an invasive mammary ductal carcinoma, in particular with lymph node involvement. In one embodiment, the patient to be treated has metastasizing breast cancer and is afflicted with skin metastases and/or lymph node metastases. In particular, the patient to be treated can have lesions at the site of the primary tumor and/or of one or more metastases. In particular, the patient may have skin lesions such as skin ulcerations, in particular skin ulcerations having a diameter of at least 2 cm, preferably at least 3 cm, at least 4 cm, at least 5 cm or at least 6 cm. The patient may also have mediastinal adenopathies caused by lymph node metastases. In particular, in the patient to be treated at least a part of the primary tumor and/or of the metastases was removed, for example by surgery and/or radiotherapy, and wherein, for example, metastases and/or a recurrent tumor are present.

In certain embodiments, the patient to be treated has a HER2 positive tumor and/or HER2 positive metastases which are estrogen receptor negative (ER−) and/or progesterone receptor negative (PgR−). Estrogen receptor negative refers to cancer wherein no estrogen receptor could be detected on the cancer cells. Progesterone receptor negative refers to cancer wherein no progesterone receptor could be detected on the cancer cells.

The HER2 positive cancer can prior to the treatment with the reduced fucose anti-HER2 antibody according to the present invention be resistant to or may have progressed after treatment with one or more anti-cancer agents such as chemotherapeutic agents and/or therapeutic antibodies, in particular one or more of the chemotherapeutic agents described herein and/or one or more of the antibodies described herein, preferably at least one or more of the high fucose anti-HER2 antibodies described herein such as trastuzumab (Herceptin) and/or pertuzumab (Omnitarg). Furthermore, the HER2 positive neoplastic disease is resistant to or has progressed following radiotherapy.

The patient to be treated may be any human patient suffering from HER2 positive cancer. Preferably, the patient is a heavily pretreated cancer patient, in particular a patient who was subject to one or more, preferably two or more, three or more, four or more, five or more or six or more cancer therapies prior to the treatment with the reduced fucose anti-HER2 antibody. Respective preceding treatments which characterize the patient to be treated were described above, it is referred to the above disclosure.

The present inventors inter alia found that the reduced fucose anti-HER2 antibody according to the invention provides an improved treatment of patients with HER2 positive cancer which is resistant to or has progressed after one or more cancer therapies regardless of the allotype of the Fcγ receptor IIIa. As was demonstrated in in vitro assays and confirmed in the clinical setting, the reduced fucose anti-HER2 antibody according to the invention has an increased ADCC activity in particular with effector cells obtained from donors having the FcγRIIIa-158F/F or F/V allotype. With these effector cells, a high fucose anti-HER2 antibody is less effective in the ADCC assay. However, the reduced fucose anti-HER2 antibody according to the invention also shows an improved treatment of patients with cancer which is resistant to common high fucose anti-HER2 antibodies for patients having the FcγRIIIa-158V/V allotype. The patient may hence have any allotype of the Fcγ receptor IIIa and will benefit from the novel treatments described herein. In particular, the patient may be homozygous for valine in amino acid position 158 of the Fcγ receptor IIIa (FcγRIIIa-158V/V), or the patient may be homozygous for phenylalanine in amino acid position 158 of the Fcγ receptor IIIa (FcγRIIIa-158F/F), or the patient may be heterozygous for valine and phenylalanine in amino acid position 158 of the Fcγ receptor IIIa (FcγRIIIa-158V/F). Thus, all FcγIIIa allotypes, including the F/F and F/V allotypes, can be treated and also patients having a low HER2 overexpression, such as HER2 1+ and HER2 2+. The clinical study using the reduced fucose anti-HER2 antibody according to the invention also showed that the treatment with said antibody causes very few adverse reactions. In particular, no clinical cardiotoxic effects were observed in the study. In contrast thereto, the commercially available high fucose anti-HER2 antibody Herceptin® is known to have cardiotoxic side effects. Hence, in particular embodiments the reduced fucose anti-HER2 antibody according to the invention causes fewer adverse reactions, preferably has a lower cardiotoxicity, than trastuzumab as used in the medicament Herceptin®.

In preferred embodiments, the reduced fucose anti-HER2 antibody is for cancer treatment of patients having congestive heart failure, symptomatic heart insufficiency, coronary heart disease, uncontrolled arrhythmias, angina pectoris, heart valve insufficiency, hypertonia, myocardial infarction, dyspnea at rest, or a risk for one or more of these diseases. Preferably patients having a left ventricular ejection fraction of 55% or less, in particular 50% or less or 45% or less can be treated with the reduced fucose anti-HER2 antibody. In particular embodiments, the reduced fucose anti-HER2 antibody is suitable for treatment of these patients in combination with one or more antracyclines such as daunorubicin, doxorubicin, epirubicin, idarubicin, valrubicin and mitoxantrone.

Furthermore, the clinical study also showed that the reduced fucose anti-HER2 antibody according to the invention is well-tolerated by the patients and causes fewer adverse reactions than Herceptin, in particular no adverse gastrointestinal reactions such as diarrhea, nausea and vomiting. Thus, preferably the reduced fucose anti-HER2 antibody is for treatment of patients for whom adverse gastrointestinal reactions are critical.

### The Composition Comprising the Reduced Fucose Anti-HER2 Antibody and Dosages

The reduced fucose anti-HER2 antibody in particular is comprised in a therapeutic composition. It preferably is a composition suitable for intravenous injection, for example an aqueous solution comprising the antibody, or a composition which can be used to prepare a composition suitable for intravenous injection, for example a lyophilized antibody composition. The composition comprising the reduced fucose anti-HER2 antibody may additionally comprise one or more further components selected from the group consisting of solvents, diluents, and excipients. The components of the composition preferably are all pharmaceutically acceptable. The composition may be a solid or fluid composition, in particular a—preferably aqueous—solution, emulsion or suspension or a lyophilized powder.

The composition preferably comprising the reduced fucose anti-HER2 antibody in a concentration in the range of from 1 mg/ml to 100 mg/ml, more preferably from 5 mg/ml to 50 mg/ml, from 10 mg/ml to 30 mg/ml or from 15 mg/ml to 25 mg/ml, in particular about 20 mg/ml.

The reduced fucose anti-HER2 antibody may be administered to the patient by any suitable administration route, preferably by intravenous injection. In preferred embodiments, the reduced fucose anti-HER2 antibody is administered in a dose in the range of from 0.5 to 15 mg, 1 to 10 mg, preferably 2 to 8 mg, more preferably 3 to 6 mg or 3 to 5 mg, most preferably about 4 mg or about 6 mg per kg body weight of the patient or less. Here, it was found that the reduced fucose anti-HER2 antibodies can be administered at lower dosages than high fucose anti-HER2 antibodies and elicit a therapeutic effect even when given as monotherapy. Therefore, advantageously lower dosages can be used. Alternatively, due to the reduced adverse side effects profile, the reduced fucose anti-HER2 antibody may also be administered in higher doses, for example in a dose in the range of from 0.2 to 30 mg, preferably 2 to 25 mg, more preferably 4 to 20 mg or 6 to 18 mg, most preferably 8 to 15 mg, in particular about 12 mg per kg body weight of the patient. In certain embodiments, the reduced fucose anti-HER2 antibody is administered in a dose per administration in the range of from 10 mg to 1250 mg, preferably from 50 mg to 1000 mg, more preferably from 100 mg to 800 mg, from 200 mg to 750 mg or from 240 to 600 mg. Higher doses of 400 to 2000 mg, preferably 500 to 1500 mg, more preferably 600 to 1000 mg are also possible. Preferably, the reduced fucose anti-HER2 antibody is administered in intervals in the range of from 1 week to 2 months, preferably from 2 weeks to 6 weeks, more preferably from 3 weeks to 4 weeks, in particular every third or fourth week. The doses described above are in particular optimized for administration every third week. Due to the high efficacy of the anti-HER2 antibodies, it is possible to prolong the administration interval, e.g. from three weeks to four weeks without having to increase the dosage. According to one embodiment, the treatment comprises administering to the patient the reduced fucose anti-HER 2 antibody in an initial dose of 1 to 10 mg, preferably 2 to 8 mg, more preferred 3 to 6 mg and administering to the patient a plurality of subsequent doses of the reduced fucose anti-HER 2 antibody in an amount which is the same or lower than the initial dose, wherein the initial dose and the subsequent dose are separated in time from each other by at least 1 week, at least 2 weeks, at least 3 weeks, preferably at least 4 weeks.

The administration of antibodies by injection, including infusion, may cause adverse reactions in the patient's body, in particular infusion related reactions (IRR). Respective effects can also occur when administering the reduced fucose anti-HER2 antibody. To reduce respective infusion related reactions, the treatment of the reduced fucose anti-HER 2 antibody may be combined with means for treatment or prevention of such infusion related reactions.

According to one embodiment of the invention, the prevention or reduction of IRR is achieved by combining the treatment of the reduced fucose anti-HER2 antibody with a pre-medication of an agent with analgesic and/or antipyretic properties. Said agent may have one or more of the following characteristics: it is a non-opioid analgesic, it is a non-salicylate analgesic, it is an aniline analgesic/aniline derivative, it is an acetanilide derivative, it is an aminophenol derivative, it is an acetylaminophenol, it is a cyclo-oxygenase inhibitor and/or it is prostaglandin inhibitor. Preferably N-(4-hydroxyphenyl)acetamide (paracetamol or acetaminophen) is used as analgesic and/or antipyretic agent. The agent preferably is administered intravenously or orally.

It was found by the inventors that such a pre-medication unexpectedly significantly reduces IRR associated with the administration of the reduced fucose anti-HER2 antibody. Hence in one aspect of the invention this pre-medication is use to prevent or treat IRR caused by the administration of reduced fucose anti-HER2 antibody. Exemplary infusion related reactions are fever, edema such as angioedema, arthralgia and shivering.

The agent with analgesic and/or antipyretic properties preferably is administered in a dose from 250 mg to 1500 mg, at least 500 mg, preferably at least 700 mg, at least 800 mg, at least 900 mg, more preferably of 1000 mg. It is preferably administered prior to administration of the reduced fucose anti-HER2 antibody, preferably in one single dose or in two or more, preferably two separate doses.

In preferred embodiments, the agent is administered 5 min to 6 h, preferably 10 min to 4 h, 15 min to 3 h or 20 min to 2 h, more preferably 30 min to 90 min, in particular 1 hour before administration of the reduced fucose anti-HER2 antibody, in particular as a single dose.

In certain preferred embodiments the agent is administered in two doses, whereas a first dose is administered at 8 h to 48 h, preferably 12 h to 36 h or 16 h to 24 h, in particular at the evening before (i.e. about 12 hours before) administration of the reduced fucose anti-HER2 antibody. The second dose is administered 5 min to 6 h, preferably 10 min to 4 h, 15 min to 3 h or 20 min to 2 h, more preferably 30 min to 90 min, in particular 1 hour before administration of the reduced fucose anti-HER2 antibody. In a particular preferred embodiment a first dose of the agent is administered the evening before the administration of the antibody and a second dose is given 1 hour before the administration of the antibody. Preferably both doses are 1000 mg of the agent. A particular preferred agent of this administration scheme is N-(4-hydroxyphenyl)acetamide.

In further embodiments, the agent is administered upon occurrence of infusion related reaction to the administration of the reduced fucose anti-HER2 antibody.

The agent with analgesic and/or antipyretic properties may be administered in combination with one or more steroids, preferably glucocorticoids, such as cortisol, cortison acetate, cloprenol, prednisone, prednisolone, deflazacort, fluocortolon, triamcinolone, betamethasone or dexamethasone, in particular methylprednisolone. The steroid preferably is administered 5 min to 4 h, more preferably, 15 min to 1 h, most preferably about 30 min before administration of the reduced fucose anti-HER2 antibody. The steroid preferably is administered in a dose of from 25 to 500 mg, more preferably from 50 to 250 mg or from 100 to 150 mg, in particular in a dose of about 125 mg.

In a particular preferred embodiment of the invention the treatment of the patient with the anti-HER2 antibody is combined with a pre-medication with N-(4-hydroxyphenyl)acetamide and methylprednisolone as follows in order to effectively reduce or prevent IRR:
a) a first dose of 1000 mg of N-(4-hydroxyphenyl)acetamide the evening before the administration of the antibody,
b) a second dose of 1000 mg of N-(4-hydroxyphenyl) 1 hour before the administration of the antibody and
c) one dose of 125 mg methylprednisolone 30 min before administration of the antibody.

In this scheme the reduced fucose anti-HER2 antibody is administered in doses described above; it is referred to the above disclosure.

In certain embodiments, no steroids are administered, preferably no steroids and no antihistamines are administered. In particular, the infusion related reactions are treated or prevented only with the agent with analgesic and/or antipyretic properties.

In another aspect, the present invention provides an agent with analgesic and/or antipyretic properties for treating or preventing infusion related reactions caused by the administration of an anti-HER2 antibody. The anti-HER2 antibody preferably is the reduced fucose anti-HER2 antibody as defined herein. The features and embodiments of the other aspects of the invention accordingly apply to this aspect of the invention.

### Methods of Treatment

In a further aspect, the present invention is directed to a method of treatment of a patient suffering from a HER2-positive neoplastic disease, comprising administering an anti-HER2 antibody having an amount of fucose in the CH2 domain of 50% or less, preferably 30% or less, more preferably 15% to 0% (reduced fucose anti-HER2 antibody) to said patient in an amount sufficient to treat the neoplastic disease.

In certain embodiments, the present invention provides a method of treatment of a human patient with a HER2 positive cancer, wherein the cancer is a metastasizing cancer, comprising administering an anti-HER2 antibody having an amount of fucose in the CH2 domain of 50% or less, preferably 30% or less, more preferably 15% to 0% (reduced fucose anti-HER2 antibody).

In certain embodiments, present invention provides a method of treatment of a patient suffering from a HER2-positive neoplastic disease, in particular HER2 positive cancer, after treatment with a high fucose anti-HER2 antibody or an anti-HER2 antibody which is not glycosylated, comprising administering a reduced fucose anti-HER2 antibody to said patient in an amount sufficient to treat the neoplastic disease. In particular, the reduced fucose anti-HER2 antibody has an amount of fucose in the CH2 domain of 50% or less and the high fucose anti-HER2 antibody has an amount of fucose in the CH2 domain of 60% or more. In preferred embodiments, prior to the treatment with the reduced fucose anti-HER2 antibody said patient has been treated with
a) at least one chemotherapeutic agent;
b) at least one anti-HER2 antibody having an amount of fucose in the CH2 domain of fucose 60% or more (high fucose anti-HER2 antibody), or at least one anti-HER2 antibody which is not glycosylated;
c) optionally radiotherapy; and
d) optionally at least one further therapeutic antibody;
   wherein the preceding treatments a), b), c) and d) occurred in any order sequentially or concurrently.

In certain embodiments, the present invention is directed to a method of treatment of a human patient with a HER2 positive cancer, wherein the HER2 positive cancer has a HER2 overexpression of level 2+ or lower, preferably level 1+, as determined by immunohistochemistry (IHC), comprising administering an anti-HER2 antibody having an amount of fucose in the CH2 domain of 50% or less, preferably 30% or less, more preferably 15% to 0% (reduced fucose anti-HER2 antibody).

All the embodiments and features described above and below also likewise apply to the methods of treatment according to the invention.

### SPECIFIC EMBODIMENTS OF THE PRESENT INVENTION

Specific and particularly preferred embodiments of the present invention will be described in the following:

### Specific Embodiments of the Treatment of Metastazing Cancer

In a first specific embodiment of said first aspect, the present invention is directed to a reduced fucose anti-HER2 antibody for treating a patient with a metastasizing HER2 positive cancer, preferably breast cancer or colon cancer, wherein the reduced fucose anti-HER2 antibody
(i) has in the CH2 domain an amount of fucose of 20% or less, an amount of bisecting GlcNAc of at least 8% and an amount of galactose of at least 65%;
(ii) comprises a heavy chain variable region comprising the amino acid sequence of SEQ ID NO: 7 or an amino acid sequence which is at least 80% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3;
(iii) comprises a light chain variable region comprising the amino acid sequence of SEQ ID NO: 8 or an amino acid sequence which is at least 80% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6;
   and wherein prior to the treatment with the reduced fucose anti-HER2 antibody said patient has been treated with
a) at least one, at least two and preferably at least three different chemotherapeutic agents; and/or
b) at least one anti-HER2 antibody having an amount of fucose in the CH2 domain of 60% or more (high fucose anti-HER2 antibody), wherein the amino acid sequences of its heavy chain variable region and light chain variable region are at least 80%, preferably at least 90% identical to those of the reduced fucose anti-HER2 antibody, preferably trastuzumab (Herceptin®);
c) optionally radiotherapy; and
d) optionally at least one further therapeutic antibody;
   wherein the preceding treatments a), b), optionally c) and optionally d) occurred in any order sequentially or concurrently. Preferably, the preceding treatments in this first embodiment included one or more of the following
(i) at least one treatment with the high fucose anti-HER2 antibody trastuzumab (Herceptin®) as monotherapy and/or at least one combination treatment with a chemotherapeutic agent, preferably a taxane such as docetaxel and vinorelbine, in particular at least one monotherapy with the high fucose anti-HER2 antibody trastuzumab (Herceptin®) and additionally at least one, preferably at least two combination treatments with the high fucose anti-HER2 antibody trastuzumab (Herceptin);
(ii) at least one treatment with at least one taxane, preferably at least two separate treatments with one, two or more different taxanes, preferably with paclitaxel and docetaxel;
(iii) at least one treatment with a platinum based chemotherapeutic agent such as cisplatin, preferably in combination with a chemotherapeutic agent such as gemcitabine;
(iv) radiotherapy, preferably as adjuvant therapy;
(v) at least one, preferably at least two, at least three or at least four treatments with a chemotherapeutic agent or a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine;
   and/or
(vi) surgical removal of at least a part of the primary tumor and/or one or more metastases.

In particular, the preceding treatments of the patient include in this first embodiment at least two, preferably at least three, at least four, at least 5 or all 6 of the treatments (i) to (vi). Preferably, the preceding treatments include at least treatments (i), (v) and (vi).

In a second specific embodiment, the present invention is directed to a reduced fucose anti-HER2 antibody for treating a patient with a metastasizing HER2 positive cancer, wherein the reduced fucose anti-HER2 antibody
(i) has in the CH2 domain an amount of fucose of 20% or less, an amount of bisecting GlcNAc of at least 8% and an amount of galactose of at least 65%, has no detectable NeuGc, has no detectable Galα2,6-coupled NeuAc and preferably, the reduced fucose anti-HER2 antibody was recombinantly produced in a human cell line;
(ii) comprises a heavy chain variable region comprising the amino acid sequence of SEQ ID NO: 7 or an amino acid sequence which is at least 80% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3;
(iii) comprises a light chain variable region comprising the amino acid sequence of SEQ ID NO: 8 or an amino acid sequence which is at least 80% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6;
(iv) is capable of inducing a stronger ADCC than trastuzumab (Herceptin®);
   and wherein prior to the treatment with the reduced fucose anti-HER2 antibody said patient has been treated with
a) at least two, preferably at least three different chemotherapeutic agents; and
b) at least one anti-HER2 antibody having an amount of fucose in the CH2 domain of 60% or more (high fucose anti-HER2 antibody), wherein the amino acid sequences of its heavy chain variable region and light chain variable region are at least 80%, preferably at least 90% identical to those of the reduced fucose anti-HER2 antibody, preferably trastuzumab (Herceptin®);
c) optionally radiotherapy; and
d) optionally at least one further therapeutic antibody;
   wherein the preceding treatments a), b), optionally c) and optionally d) occurred in any order sequentially or concurrently and wherein the reduced fucose anti-HER2 antibody is for the treatment of a metastases selected from skin metastases, in particular ulcerating skin metastases, lymphnode metastases and visceral metastases, in particular lung or liver metastases. Preferred preceding treatments were described above in conjunction with the first specific embodiment, it is referred to the respective disclosure.

The patient that is treated in the first or second specific embodiment may have the following characteristics:
(i) the patient is homozygous for valine in amino acid position 158 of the Fcγ receptor IIIa (FcγRIIIa-158V/V); or
(ii) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F).

In particular, the reduced fucose anti-HER2 antibody of the first and second specific embodiment can be used for the treatment of patients irrespective of their FcγRIIIa allotype. In the first and second specific embodiment, the HER2 positive cancer and/or metastasis may have a HER2 overexpression of level 2+ or lower, preferably 1+ or lower, as determined by immunohistochemistry. Preferably, the HER2 positive cancer and/or metastasis is positive for HER2 gene amplification as determined by FISH or CISH. According to one aspect the patient to be treated in the first or second specific embodiment is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F) and optionally additionally the HER2 positive cancer and/or metastasis has a HER2 overexpression of level 2+ or lower, preferably 1+ or lower, as determined by immunohistochemistry.

Suitable and preferred dosages of the reduced fucose anti-HER2 antibody and suitable and preferred premedication schedules were described above; it is referred to the above disclosure which also applied to the first and second specific embodiment. The reduced fucose anti-HER2 antibody according to the first and second specific embodiment may be for use as monotherapy or as combination therapy. Embodiments were described above and it is referred to the respective disclosure.

As described above, the present invention is directed to a method of treatment of a human patient with a HER2 positive cancer, wherein the cancer is a metastasizing cancer, comprising administering an anti-HER2 antibody having an amount of fucose in the CH2 domain of 50% or less, preferably 30% or less, more preferably 15% to 0% (reduced fucose anti-HER2 antibody).

All the embodiments and features described above or below also likewise apply to the methods of treatment according to the invention.

As described above, in a certain aspect, the present invention is directed to an anti-HER2 antibody having an amount of fucose in the CH2 domain of 50% or less (reduced fucose anti-HER2 antibody) for treating a human patient with a HER2 positive cancer, wherein the cancer is a metastasizing cancer.

Preferably, the anti-HER2 antibody is for the treatment of metastases, wherein the metastases include one or more of skin metastases, in particular ulcerating skin metastases, visceral metastases, in particular lung and/or liver metastases and lymph node metastases. According to one embodiment, the patient has one or more visceral metastases, in particular lung and/or liver metastases. Preferably, the HER2 positive cancer has one or more of the following characteristics: (i) it is breast cancer, preferably metastasizing breast cancer; (ii) it is an invasive mammary ductal carcinoma, preferably with lymph node involvement; (iii) it is associated with lymph node metastases and/or skin metastases, in particular is associated with mediastinal adenopathies caused by lymph node metastases and/or skin ulcerations caused by skin metastases; (iv) it is associated with visceral metastases, in particular lung and/or liver metastases; (v) it is selected from the group consisting of colon cancer, salivary gland cancer such as parotid gland carcinoma, lung cancer such as non-small cell lung carcinoma, and bronchial cancer. According to one embodiment, the HER2 positive metastases have one or more of the following characteristics: (i) estrogen receptor negative (ER−) and/or progesterone receptor negative (PgR−); (ii) a HER2 overexpression of at least level 1+, preferably level 2+ or level 3+, as determined by immunohistochemistry; (iii) a HER2 overexpression of level 2+ or lower, preferably level 1+ or lower, as determined by immunohistochemistry; (v) they are positive for HER2 gene amplification as determined by fluorescence in situ hybridization (FISH) or chromogen in situ hybridization (CISH).

According to one embodiment, the anti-HER2 antibody is for (i) the treatment of a primary tumor; (ii) the treatment of a recurrent tumor; (iii) inhibition of tumor growth; (iv) the treatment of metastases, including skin metastases, in particular ulcerating skin metastases, lymph node metastases, visceral metastases, in particular lung and/or liver metastases; and/or (v) the treatment of lesions caused by a metastasis, in particular skin lesions or lymph node lesions, more particularly skin ulcers. Preferably, the treatment with the reduced fucose anti-HER2 antibody results in one or more of the following: (i) a prevention of further metastases; (ii) a reduction of lesions caused by one or more metastases, in particular skin ulcers; (iii) reduction of the number of metastases. According to one embodiment, prior to the treatment with the reduced fucose anti-HER2 antibody said patient has been treated with a) at least one chemotherapeutic agent; and/or b) at least one anti-HER2 antibody having an amount of fucose in the CH2 domain of 60% or more (high fucose anti-HER2 antibody), or at least one anti-HER2 antibody which is not glycosylated; c) optionally radiotherapy; and d) optionally at least one further therapeutic antibody; wherein the preceding treatments a), b), optionally c) and optionally d) occurred in any order sequentially or concurrently. According to one embodiment, the neoplastic disease reoccurred or progressed following the preceding treatments. As is shown in the examples, the anti-HER2 antibody according to the invention is particularly effective in successfully treating pretreated patients, including heavily pretreated patients, wherein previous treatments failed or where the cancer reoccurred or further metastases developed.

According to one embodiment, prior to the treatment with the reduced fucose anti-HER2 antibody the patient has been treated with at least two, preferably at least three, at least four, or at least five different anti-cancer agents, in particular chemotherapeutic agents either in mono- or combination therapy. Preferably, the preceding treatments include one or more, preferably at least two, at least three, at least four or at least five, most preferably all of the following treatments: (i) at least one treatment with trastuzumab (Herceptin®) as monotherapy; (ii) at least one treatment with trastuzumab (Herceptin®) in combination with a chemotherapeutic agent, preferably in combination with a taxane such as docetaxel and vinorelbine; (iii) at least one treatment with a taxane, preferably at least two separate treatments with different taxanes, preferably with paclitaxel and docetaxel; (iv) at least one treatment with a platinum based chemotherapeutic agent such as cisplatin, preferably in combination with a chemotherapeutic agent such as gemcitabine; (v) radiotherapy, preferably as adjuvant therapy; (vi) at least one treatment with a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine. According to one embodiment, the preceding treatment of the patient involved cancer surgery, preferably a surgical removal of the primary tumor and/or of metastases. According to a preferred embodiment, the HER2 positive cancer is resistant to or has progressed after treatment with at least one chemotherapeutic agent and/or is resistant to or has progressed after treatment with high fucose trastuzumab (Herceptin®) and/or high fucose pertuzumab (Omnitarg). According to one embodiment, the treatment with the reduced fucose anti-HER2 antibody is for adjuvant treatment, for neoadjuvant treatment, for neoadjuvant-adjuvant treatment or for palliative treatment. Preferably, the reduced fucose anti-HER2 antibody is repeatedly administered to the patient and a therapeutic effect is obtained at least after the second administration of the reduced fucose anti-HER2 antibody, preferably already after the first administration of the reduced fucose anti-HER2 antibody. Preferably, the therapeutic effect includes a reduction of skin lesions, in particular ulcerating skin lesions, a reduction of mediastinal adenopathies and/or a reduction of visceral metastases, in particular lung and/or liver metastases.

Preferably, the reduced fucose anti-HER2 antibody has an amount of fucose in the CH2 domain of 20% or less, 15% or less, 10% or less, 5% or less or 0%, in particular in the range of from 2% to 20%, from 3% to 15% or from 5% to 10%. Preferably, the reduced fucose anti-HER2 antibody has one or more, preferably all of the following glycosylation characteristics in the CH2 domain: (i) an amount of bisecting GlcNAc of at least 8%; (ii) an amount of galactose of at least 65%; (iii) optionally no detectable NeuGc; (iv) optionally no detectable Galα1,3-Gal; (v) optionally detectable α2,6-coupled NeuAc. Preferably, the reduced fucose anti-HER2 antibody has one or more, preferably at least two, more preferably all of the following characteristics: (i) it comprises a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3; (ii) it comprises a heavy chain variable region comprising the amino acid sequence of SEQ ID NO: 7 or an amino acid sequence which is at least 80% identical thereto; (iii) it comprises a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6; (iv) it comprises a light chain variable region comprising the amino acid sequence of SEQ ID NO: 8 or an amino acid sequence which is at least 80% identical thereto; (v) it shows cross-specificity with the antibody trastuzumab; (vi) it comprises heavy chain and light chain amino acid sequences which are at least 90% identical to the amino acid sequences of the antibody trastuzumab; (vii) it is equivalent to the antibody trastuzumab in binding and Fv mediated anti-tumor response; (viii) it was recombinantly produced in a human cell line. Preferably, the reduced fucose anti-HER2 antibody is capable of inducing a stronger ADCC than a corresponding high fucose anti-HER2 antibody, which preferably is trastuzumab (Herceptin®).

According to one embodiment, the high fucose anti-HER2 antibody, which preferably is trastuzumab (Herceptin®), has an amount of fucose in the CH2 domain of 70% or more, in particular 80% or more. Preferably, the high fucose anti-HER2 antibody used in the pretreatment has one or more, preferably at least three of the following characteristics: (i) it is an IgG antibody; (ii) it shows cross-specificity with the reduced fucose anti-HER2 antibody; (iii) it is capable of specifically binding to the same epitope as the reduced fucose anti-HER2 antibody; (iv) the amino acid sequences of its heavy chain variable region and light chain variable region are at least 80%, at least 90% or at least 95%, more preferably 100% identical to those of the reduced fucose anti-HER2 antibody; (v) it is the antibody trastuzumab (Herceptin®); (vi) it is capable of specifically binding to HER2, wherein the epitope of the high fucose anti-HER2 antibody is different from the epitope of the reduced fucose anti-HER2 antibody; and/or (vii) it is the antibody pertuzumab (Omnitarg).

According to one embodiment, the treatment with the reduced fucose anti-HER2 antibody is a monotherapy. Alternatively, the treatment with the reduced fucose anti-HER2 antibody is a combination therapy, in particular in combination with (i) at least one chemotherapeutic agent; and/or (ii) at least one further therapeutic antibody which is different from the reduced fucose anti-HER2 antibody; and/or (iv) cancer surgery and/or radiotherapy.

As described above, the patients to be treated with the reduced fucose anti-HER2 antibody may have received prior cancer treatments. According to one embodiment, the prior treatment involved at least one chemotherapeutic agent. Here, the at least one chemotherapeutic agent used in the pretreatment of the patient may be selected from the group consisting of cyclophosphamide; lapatinib; capecitabine; cytarabine; vinorelbine; bevacizumab; gemcitabine; maytansine; anthracyclines such as daunorubicin, doxorubicin, epirubicin, idarubicin, valrubicin and mitoxantrone; taxanes such as paclitaxel (Taxol), docetaxel (Taxotere) and SB-T-1214; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues.

Preferably, the preceding treatment of the patient involved the use of at least one therapeutic antibody different from the reduced fucose anti-HER2 antibody and which in particular is selected from the group consisting of anti-HER2 antibodies which differ in their mode of action from the reduced fucose anti-HER2 antibody, in particular pertuzumab, anti-EGFR antibodies such as cetuximab (Erbitux), panitumomab (Vectibix) and nimotuzumab (Theraloc); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).

According to one embodiment, the treatment with reduced fucose anti-HER2 antibody is a combination therapy with at least one different anti-cancer agent, wherein the anti-cancer agent is selected from the group consisting of (i) chemotherapeutic agents, wherein the chemotherapeutic agent preferably is a taxane, and (ii) anti-cancer therapeutic antibodies, wherein the therapeutic antibody preferably is an anti-HER2 antibody which differs in its mode of action from the reduced fucose anti-HER2 antibody such as pertuzumab if the reduced fucose anti-HER2 antibody corresponds to trastuzumab, anti-EGFR antibodies such as cetuximab (Erbitux) and/or an anti-VEGF antibody such as bevacizumab (Avastin).

Preferably, the reduced fucose anti-HER2 antibody is administered in an amount of from 1 to 10 mg/kg body weight of the patient every first, second, third or fourth week or less frequently; preferably in an amount of from 2 to 8 mg/kg body weight of the patient every third week or less frequently. Preferably, the reduced fucose anti-HER2 antibody has a higher therapeutic efficacy than the high fucose anti-HER2 antibody when the reduced fucose anti-HER2 antibody is administered at the same dose but less frequently than the high fucose anti-HER2 antibody or when the reduced fucose anti-HER2 antibody is administered at the same frequency but at a lower dose than the high fucose anti-HER2 antibody.

As discussed above, the improved therapeutic efficacy allows to treat different patients that could not or could less effectively be treated with prior art anti-HER2 antibodies. Here, different options exist: (i) the patient is homozygous for valine in amino acid position 158 of the Fcγ receptor IIIa (FcγRIIIa-158V/V); (ii) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F); (iii) the reduced fucose anti-HER2 antibody is for treatment of patients irrespective of their FcγRIIIa allotype; or (iv) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F) and wherein the HER2 positive cancer and/or metastasis has a HER2 overexpression of level 2+ or lower, preferably 1+ or lower, as determined by immunohistochemistry and wherein preferably, the HER2 positive cancer and/or metastasis is positive for HER2 gene amplification as determined by FISH or CISH.

In certain embodiments, the treatment with the reduced fucose anti-HER2 antibody is combined with a pre-medication of the patient with an agent with analgesic and/or antipyretic properties, in particular with N-(4-hydroxyphenyl)acetamide.

Preferably, the pre-medication comprises at least two separate doses of the agent with analgesic and/or antipyretic properties, whereas the first dose is given 8 h to 48 before the administration of the reduced fucose anti-HER2 antibody and the second dose is given 5 min to 6 hours before the administration of the reduced fucose anti-HER2 antibody. Preferably, each of the doses contains 250 mg and 1500 mg, in particular 1000 mg of the agent with analgesic and/or antipyretic properties. Preferably, the pre-medication further comprises the administration of a steroid, preferably a glucocorticoid, in particular methylprednisolone. Preferably, the steroid is administered 5 min to 4 h, in particular 30 min before the administration of the antibody. Preferably, the pre-medication comprises, or consists of, the following steps: a) a first dose of 1000 mg of N-(4-hydroxyphenyl) acetamide the evening before the administration of the antibody, b) a second dose of 1000 mg of N-(4-hydroxyphenyl) 1 hour before the administration of the reduced fucose anti-HER2 antibody; and c) one dose of 125 mg methylprednisolone 30 min before administration of the reduced fucose anti-HER2 antibody.

In certain embodiments, the present invention provides an analgesic and/or antipyretic agent for treating or preventing infusion related reactions caused by the administration of reduced fucose anti-HER2 antibodies according to the pre-medication as described above.

### Specific Embodiments of the Treatment of Pretreated Patients

In the following, specific embodiments of the present invention according to the second aspect concerning the treatment of patients that have received prior cancer treatments are listed. All features and embodiments described herein above also apply to and can be combined with the following embodiments.
1. An anti-HER2 antibody having an amount of fucose in the CH2 domain of 50% or less (reduced fucose anti-HER2 antibody) for treating a patient with a HER2 positive neoplastic disease, in particular HER2 positive cancer, wherein prior to the treatment with the reduced fucose anti-HER2 antibody said patient has been treated with
   a) at least one chemotherapeutic agent; and
   b) at least one anti-HER2 antibody having an amount of fucose in the CH2 domain of 60% or more (high fucose anti-HER2 antibody), or at least one anti-HER2 antibody which is not glycosylated;
   c) optionally radiotherapy;
   d) optionally at least one further therapeutic antibody;
   wherein the preceding treatments a), b), optionally c) and optionally d) occurred in any order sequentially or concurrently.
2. The anti-HER2 antibody according to embodiment 1, wherein the neoplastic disease reoccurred or progressed following the preceding treatments.
3. The anti-HER2 antibody according to embodiment 1 or 2, wherein prior to the treatment with the reduced fucose anti-HER2 antibody the patient has been treated with at least two, preferably at least three, at least four, or at least five different chemotherapeutic agents either in mono- or combination therapy.
4. The anti-HER2 antibody according to any one of embodiments 1 to 3, wherein the preceding treatments include one or more, preferably at least two, at least three, at least four or at least five or all of the following treatments:
   (i) at least one treatment with trastuzumab (Herceptin®) as monotherapy;
   (ii) at least one treatment with trastuzumab (Herceptin®) in combination with a chemotherapeutic agent, preferably in combination with a taxane such as docetaxel and vinorelbine;
   (iii) at least one treatment with a taxane, preferably at least two separate treatments with different taxanes, preferably with paclitaxel and docetaxel;
   (iv) at least one treatment with a platinum based chemotherapeutic agent such as cisplatin, preferably in combination with a chemotherapeutic agent such as gemcitabine;
   (v) radiotherapy, preferably as adjuvant therapy;
   (vi) at least one treatment with a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine.
5. The anti-HER2 antibody according to any one of embodiments 1 to 4, wherein the preceding treatment of the patient involved cancer surgery, preferably a surgical removal of the primary tumor and/or of metastases.
6. The anti-HER2 antibody according to any one of embodiments 1 to 5, wherein the HER2 positive cancer is a metastasizing cancer.
7. The anti-HER2 antibody according to embodiment 6, wherein the metastases include one or more of skin metastases, visceral metastases, in particular lung and/or liver metastases and lymph node metastases.
8. The anti-HER2 antibody according to embodiment 7, wherein the patient has one or more ulcerating skin metastases.
9. The anti-HER2 antibody according to any one of embodiments 1 to 8, for the treatment of a HER2 positive cancer having one or more of the following characteristics:
   (i) it is breast cancer, preferably metastasizing breast cancer;
   (ii) it is an invasive mammary ductal carcinoma, preferably with lymph node involvement;
   (iii) it is associated with lymph node metastases and/or skin metastases, in particular is associated with mediastinal adenopathies caused by lymph node metastases and/or skin ulcerations caused by skin metastases;
   (iv) it is associated with visceral metastases, in particular lung and/or liver metastases.
10. The anti-HER2 antibody according to any one of embodiments 1 to 9, for the treatment of a HER2 positive tumor and/or metastases having one or more of the following characteristics:
   (i) estrogen receptor negative (ER−) and/or progesterone receptor negative (PgR−);
   (ii) a HER2 overexpression of at least level 1+, preferably level 2+ or level 3+, as determined by immunohistochemistry;
   (iii) a HER2 overexpression of level 2+ or lower, preferably level 1+ or lower, as determined by immunohistochemistry;
   (v) it is positive for HER2 gene amplification as determined by fluorescence in situ hybridization (FISH) or chromogen in situ hybridization (CISH).
11. The anti-HER2 antibody according to any one of embodiments 1 to 10, wherein the HER2 positive cancer is resistant to or has progressed after treatment with at least one chemotherapeutic agent and/or is resistant to or has progressed after treatment with high fucose trastuzumab (Herceptin) and/or high fucose pertuzumab (Omnitarg).
12. The anti-HER2 antibody according to any one of embodiments 1 to 11, for
   (i) the treatment of a primary tumor;
   (ii) the treatment of a recurrent tumor;
   (iii) for inhibition of tumor growth;
   (iv) the treatment of metastases, including skin metastases, in particular ulcerating skin metastases, lymph node metastases, visceral metastases, in particular lung and/or liver metastases; and/or
   (v) the treatment of lesions caused by a tumor or a metastasis, in particular skin lesions or lymph node lesions, more particularly skin ulcers.
13. The anti-HER2 antibody according to any one of embodiments 1 to 12, wherein the treatment with the reduced fucose anti-HER2 antibody results in one or more of the following:
   (i) inhibition of tumor growth;
   (ii) reduction of tumor size;
   (iii) prevention of further metastases;
   (iv) reduction of lesions caused by the primary tumor and/or one or more metastases, in particular skin ulcers;
   (v) reduction of the number of metastases;
   (vii) increase in progression-free survival; and/or
   (viii) increase in lifespan.
14. The anti-HER2 antibody according to any one of embodiments 1 to 13, wherein the treatment with the reduced fucose anti-HER2 antibody is for adjuvant treatment, for neoadjuvant treatment, for neoadjuvant-adjuvant treatment or for palliative treatment.
15. The anti-HER2 antibody according to any one of embodiments 1 to 14, wherein the reduced fucose anti-HER2 antibody is repeatedly administered to the patient and wherein a therapeutic effect is obtained at least after the second administration of the reduced fucose anti-HER2 antibody, preferably already after the first administration of the reduced fucose anti-HER2 antibody.
16. The anti-HER2 antibody according to embodiment 15, wherein the therapeutic effect includes a reduction of skin lesions, in particular ulcerating skin lesions, a reduction of mediastinal adenopathies and/or a reduction of visceral metastases, in particular lung and/or liver metastases.
17. The anti-HER2 antibody according to any one of embodiments 1 to 16, having an amount of fucose in the CH2 domain of 20% or less, 15% or less, 10% or less, 5% or less or 0%, preferably in the range of from 2% to 20%, from 3% to 15% or from 5% to 10%.
18. The anti-HER2 antibody according to any one of embodiments 1 to 17, having one or more, preferably all of the following glycosylation characteristics in the CH2 domain:
   (i) an amount of bisecting GlcNAc of at least 8%;
   (ii) an amount of galactose of at least 65%;
   (iii) optionally no detectable NeuGc;
   (iv) optionally no detectable Galα1,3-Gal;
   (v) optionally detectable α2,6-coupled NeuAc.
19. The anti-HER2 antibody according to any one of embodiments 1 to 18, having one or more, preferably at least two, more preferably all of the following characteristics:
   (i) it comprises a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3;
   (ii) it comprises a heavy chain variable region comprising the amino acid sequence of SEQ ID NO: 7 or an amino acid sequence which is at least 80% identical thereto;
   (iii) it comprises a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6;
   (iv) it comprises a light chain variable region comprising the amino acid sequence of SEQ ID NO: 8 or an amino acid sequence which is at least 80% identical thereto;
   (v) it shows cross-specificity with the antibody trastuzumab;
   (vi) it comprises heavy chain and light chain amino acid sequences which are at least 90% identical to the amino acid sequences of the antibody trastuzumab;
   (vii) it is equivalent to the antibody trastuzumab in binding and Fv mediated anti-tumor response;
   (ix) it was recombinantly produced in a human cell line.
20. The anti-HER2 antibody according to any one of embodiments 1 to 19, being capable of inducing a stronger ADCC than the corresponding high fucose anti-HER2 antibody.
21. The anti-HER2 antibody according to any one of embodiments 1 to 20, wherein the high fucose anti-HER2 antibody has an amount of fucose in the CH2 domain of 70% or more.
22. The anti-HER2 antibody according to any one of embodiments 1 and 21, wherein the high fucose anti-HER2 antibody used in the pretreatment has one or more, preferably at least three of the following characteristics:
   (i) it is an IgG antibody;
   (ii) it shows cross-specificity with the reduced fucose anti-HER2 antibody;
   (iii) it is capable of specifically binding to the same epitope as the reduced fucose anti-HER2 antibody;
   (iv) the amino acid sequences of its heavy chain variable region and light chain variable region are at least 80%, at least 90% or at least 95%, more preferably 100% identical to those of the reduced fucose anti-HER2 antibody;
   (v) it is the antibody trastuzumab (Herceptin®);
   (vi) it is capable of specifically binding to HER2, wherein the epitope of the high fucose anti-HER2 antibody is different from the epitope of the reduced fucose anti-HER2 antibody; and/or
   (vii) it is the antibody pertuzumab (Omnitarg).
23. The anti-HER2 antibody according to any one of embodiments 1 to 22, wherein the treatment with reduced fucose anti-HER2 antibody is a monotherapy.
24. The anti-HER2 antibody according to any one of embodiments 1 to 23, wherein the treatment with reduced fucose anti-HER2 antibody is a combination therapy, in particular in combination with
   (i) at least one chemotherapeutic agent; and/or
   (ii) at least one further therapeutic antibody which is different from the reduced fucose anti-HER2 antibody; and/or
   (iv) cancer surgery and/or radiotherapy.
25. The anti-HER2 antibody according to any one of embodiments 1 to 24, wherein
   a) the at least one chemotherapeutic agent used in the pretreatment of the patient is selected from the group consisting of cyclophosphamide; lapatinib; capecitabine; cytarabine; vinorelbine; bevacizumab; gemcitabine; maytansine; anthracyclines such as daunorubicin, doxorubicin, epirubicin, idarubicin, valrubicin and mitoxantrone; taxanes such as paclitaxel (Taxol), docetaxel (Taxotere) and SB-T-1214; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues; and/or
   b) wherein the treatment with reduced fucose anti-HER2 antibody is a combination therapy with at least one different anti-cancer agent, wherein the anti-cancer agent is selected from the group consisting of (i) chemotherapeutic agents, wherein the chemotherapeutic agent preferably is a taxane, and (ii) anti-cancer therapeutic antibodies, wherein the therapeutic antibody preferably is an anti-HER2 antibody which differs in its mode of action from the reduced fucose anti-HER2 antibody such as pertuzumab if the reduced fucose anti-HER2 antibody corresponds to trastuzumab, anti-EGFR antibodies such as cetuximab (Erbitux) and/or an anti-VEGF antibody such as bevacizumab (Avastin).
26. The anti-HER2 antibody according to any one of embodiments 1 to 25, wherein the preceding treatment of the patient involved the use of at least one therapeutic antibody different from the reduced fucose anti-HER2 antibody and which in particular is selected from the group consisting of anti-HER2 antibodies which differ in their mode of action from the reduced fucose anti-HER2 antibody, in particular pertuzumab, anti-EGFR antibodies such as cetuximab (Erbitux), panitumomab (Vectibix) and nimotuzumab (Theraloc); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).
27. The anti-HER2 antibody according to any one of embodiments 1 to 26, for administration of the reduced fucose anti-HER2 antibody in an amount of from 1 to 10 mg/kg body weight of the patient every first, second, third or fourth week or less frequently; preferably in an amount of from 2 to 5 mg/kg body weight of the patient every third week or less frequently.
28. The anti-HER2 antibody according to any one of embodiments 1 to 27, wherein the reduced fucose anti-HER2 antibody has a higher therapeutic efficacy than the high fucose anti-HER2 antibody when the reduced fucose anti-HER2 antibody is administered at the same dose but less frequently than the high fucose anti-HER2 antibody or when the reduced fucose anti-HER2 antibody is administered at the same frequency but at a lower dose than the high fucose anti-HER2 antibody.
29. The anti-HER2 antibody according to any one of embodiments 1 to 28, wherein:
   (i) the patient is homozygous for valine in amino acid position 158 of the Fcγ receptor IIIa (FcγRIIIa-158V/V);
   (ii) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F);
   (iii) the reduced fucose anti-HER2 antibody is for treatment of patients irrespective of their FcγRIIIa allotype; or
   (iv) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F) and wherein the HER2 positive cancer and/or metastasis has a HER2 overexpression of level 2+ or lower, preferably 1+ or lower, as determined by immunohistochemistry and wherein preferably, the HER2 positive cancer and/or metastasis is positive for HER2 gene amplification as determined by FISH or CISH.
30. The anti-HER2 antibody according to any one of embodiments 1 to 29, wherein the treatment of the anti-HER2 antibody is combined with a pre-medication of the patient with an agent with analgesic and/or antipyretic properties, in particular with N-(4-hydroxyphenyl)acetamide.
31. The anti-HER2 antibody according to embodiment 30, wherein the pre-medication comprises at least two separate doses of the agent with analgesic and/or antipyretic properties, whereas the first dose is given 8 h to 48 before the administration of the antibody and the second dose is given 5 min to 6 hours before the administration of the antibody.
32. The anti-HER2 antibody according to embodiment 30, wherein the each of the doses contains 250 mg and 1500 mg, in particular 1000 mg of the agent with analgesic and/or antipyretic properties.
33. The anti-HER2 antibody according to one of the embodiments 30 to 32, wherein the pre-medication further comprises the administration of a steroid, preferably a glucocorticoid, in particular methylprednisolone.
34. The anti-HER2 antibody according to embodiment 33, wherein the steroid is administered 5 min to 4 h, in particular 30 min before the administration of the antibody.
35. The anti-HER2 antibody according to one of the embodiments 30 to 34, wherein the pre-medication comprises, or consists of, the following steps:
   a) a first dose of 1000 mg of N-(4-hydroxyphenyl) acetamide the evening before the administration of the antibody,
   b) a second dose of 1000 mg of N-(4-hydroxyphenyl) 1 hour before the administration of the antibody and
   c) one dose of 125 mg methylprednisolone 30 min before administration of the antibody.
36. The anti-HER2 antibody according to any one of embodiments 1 to 35, for the treatment of a HER2 positive cancer which is selected from the group consisting of colon cancer, salivary gland cancer such as parotid gland carcinoma, lung cancer such as non-small cell lung carcinoma, and bronchial cancer.
37. An analgesic and/or antipyretic agent for treating or preventing infusion related reactions caused by the administration of a composition comprising anti-HER2 antibodies according to the pre-medication of any of the embodiments 30 to 35.

In a further aspect, the present invention is directed to a method of treatment of a patient suffering from a HER2-positive neoplastic disease, in particular HER2 positive cancer after treatment with a high fucose anti-HER2 antibody, comprising administering a reduced fucose anti-HER2 antibody to said patient in an amount sufficient to treat the neoplastic disease. In particular, the reduced fucose anti-HER2 antibody has an amount of fucose in the CH2 domain of 50% or less and the high fucose anti-HER2 antibody has an amount of fucose in the CH2 domain of 60% or more. In preferred embodiments, prior to the treatment with the reduced fucose anti-HER2 antibody said patient has been treated with
a) at least one chemotherapeutic agent;
b) at least one anti-HER2 antibody having an amount of fucose in the CH2 domain of fucose 60% or more (high fucose anti-HER2 antibody), or at least one anti-HER2 antibody which is not glycosylated;
c) optionally radiotherapy; and
d) optionally at least one further therapeutic antibody;
   wherein the preceding treatments a), b), c) and d) occurred in any order sequentially or concurrently.

All the embodiments and features described above also likewise apply to the methods of treatment according to the invention.

Specific Embodiments of the Treatment of Cancer with Low HER2 Expression

In the following, specific embodiments of the present invention according to the third aspect concerning the treatment of cancer with low HER2 expression are listed. All features and embodiments described herein above also apply to and can be combined with the following embodiments.
1. An anti-HER2 antibody having an amount of fucose in the CH2 domain of 50% or less (reduced fucose anti-HER2 antibody) for treating a patient with a HER2 positive neoplastic disease, in particular a HER2 positive cancer, wherein the HER2 positive tumor has a HER2 overexpression of level 2+ or lower, preferably level 1+, as determined by immunohistochemistry (IHC).
2. The anti-HER2 antibody according to embodiment 1, wherein the HER2 positive neoplastic disease is a metastasizing cancer.
3. The anti-HER2 antibody according to embodiment 1 or 2, wherein:
   (i) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F); or
   (ii) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F) and wherein the HER2 positive cancer and/or metastasis has a HER2 overexpression of level 2+ or lower, preferably 1+ or lower, as determined by immunohistochemistry and wherein the HER2 positive cancer and/or metastasis is positive for HER2 gene amplification as determined by FISH or CISH.
4. The anti-HER2 antibody according to any one of the embodiments 1 to 3, wherein prior to the treatment with the reduced fucose anti-HER2 antibody said patient has been treated with
   a) at least one chemotherapeutic agent; and/or
   b) at least one anti-HER2 antibody having an amount of fucose in the CH2 domain of 60% or more (high fucose anti-HER2 antibody), or at least one anti-HER2 antibody which is not glycosylated;

c) optionally radiotherapy; and
d) optionally at least one further therapeutic antibody;
wherein the preceding treatments a), b), optionally c) and optionally d) occurred in any order sequentially or concurrently.
5. The anti-HER2 antibody according to embodiment 4, wherein the HER2 positive cancer reoccurred or progressed following the preceding treatments.
6. The anti-HER2 antibody according to embodiment 4 or 5, wherein prior to the treatment with the reduced fucose anti-HER2 antibody the patient has been treated with at least two, preferably at least three, at least four, or at least five different anti-cancer agents, in particular chemotherapeutic agents and/or therapeutic antibodies either in mono- or combination therapy.
7. The anti-HER2 antibody according to any one of embodiments 4 to 6, wherein the preceding treatments include one or more, preferably at least two, at least three, at least four or at least five or all of the following treatments:
   (i) at least one treatment with trastuzumab (Herceptin®) as monotherapy;
   (ii) at least one treatment with trastuzumab (Herceptin®) in combination with a chemotherapeutic agent, preferably in combination with a taxane such as docetaxel and vinorelbine;
   (iii) at least one treatment with a taxane, preferably at least two separate treatments with different taxanes, preferably with paclitaxel and docetaxel;
   (iv) at least one treatment with a platinum based chemotherapeutic agent such as cisplatin, preferably in combination with a chemotherapeutic agent such as gemcitabine;
   (v) radiotherapy, preferably as adjuvant therapy;
   (vi) at least one treatment with a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine.
8. The anti-HER2 antibody according to any one of embodiments 4 to 7, wherein the preceding treatment of the patient involved cancer surgery, preferably a surgical removal of the primary tumor and/or of metastases.
9. The anti-HER2 antibody according to any one of embodiments 1 to 8, wherein the cancer is selected from breast cancer, gastric cancer, carcinomas, colon cancer, transitional cell carcinoma, bladder cancer, urothelial tumors, uterine cancer, advanced esophageal adenocarcinomas, gastric adenocarcinomas or gastroesophageal junction adenocarcinomas, ovarian cancer, lung cancer, lung adenocarcinoma, endometrial cancer, kidney cancer, pancreatic cancer, thyroid cancer, colorectal cancer, prostate cancer, cancer of the brain, cervical cancer, intestinal cancer and liver cancer, preferably colon cancer, salviary gland cancer such as parotid gland carcinoma, lung cancer such as non-small cell lung carcinoma, and bronchial cancer, and in particular metastatic forms of the foregoing.
10. The anti-HER2 antibody according to one or more of embodiments 2 to 9, wherein the metastases include one or more of skin metastases, visceral metastases, in particular lung and/or liver metastases and lymph node metastases.
11. The anti-HER2 antibody according to embodiment 10, wherein the patient has one or more ulcerating skin metastases.
12. The anti-HER2 antibody according to any one of embodiments 1 to 11, for the treatment of a HER2 positive cancer having one or more of the following characteristics:
   (i) it is breast cancer, preferably metastasizing breast cancer;
   (ii) it is an invasive mammary ductal carcinoma, preferably with lymph node involvement;
   (iii) it is a colon cancer;
   (iv) it is bladder cancer;
   (v) it is associated with lymph node metastases and/or skin metastases, in particular is associated with mediastinal adenopathies caused by lymph node metastases and/or skin ulcerations caused by skin metastases;
   (vi) it is associated with visceral metastases, in particular lung and/or liver metastases.
13. The anti-HER2 antibody according to any one of embodiments 1 to 12, for the treatment of a HER2 positive tumor and/or metastases having one or more of the following characteristics:
   (i) estrogen receptor negative (ER−) and/or progesterone receptor negative (PgR−)
   (ii) they are positive for HER2 gene amplification as determined by fluorescence in situ hybridization (FISH) or chromogen in situ hybridization (CISH).
14. The anti-HER2 antibody according to any one of embodiments 4 to 13, wherein the HER2 positive cancer is resistant to or has progressed after treatment with at least one chemotherapeutic agent and/or is resistant to or has progressed after treatment with high fucose trastuzumab (Herceptin) and/or high fucose pertuzumab (Omnitarg).
15. The anti-HER2 antibody according to any one of embodiments 1 to 14, for
   (i) the treatment of a primary tumor;
   (ii) the treatment of a recurrent tumor;
   (iii) for the inhibition of tumor growth;
   (iv) for the treatment of metastases, including skin metastases, in particular ulcerating skin metastases, lymph node metastases, visceral metastases, in particular lung and/or liver metastases; and/or
   (v) lesions caused by a tumor or a metastasis, in particular skin lesions or lymph node lesions, more particularly skin ulcers.
16. The anti-HER2 antibody according to any one of embodiments 1 to 15, wherein the treatment with the reduced fucose anti-HER2 antibody results in one or more of the following:
   (i) inhibition of tumor growth;
   (ii) reduction of tumor size;
   (iii) prevention of further metastases;
   (iv) reduction of lesions caused by the primary tumor and/or one or more metastases, in particular skin ulcers;
   (v) reduction of the number of metastases;
   (vii) increase in progression-free survival; and/or
   (viii) increase in lifespan.
17. The anti-HER2 antibody according to any one of embodiments 1 to 16, wherein the treatment with the reduced fucose anti-HER2 antibody is for adjuvant treatment, for neoadjuvant treatment, for neoadjuvant-adjuvant treatment or for palliative treatment.
18. The anti-HER2 antibody according to any one of embodiments 1 to 17, wherein the reduced fucose anti-HER2 antibody is repeatedly administered to the patient and wherein a therapeutic effect is obtained at least after the second administration of the reduced fucose anti-HER2 antibody, preferably already after the first administration of the reduced fucose anti-HER2 antibody.
19. The anti-HER2 antibody according to embodiment 18, wherein the therapeutic effect includes a reduction of skin lesions, in particular ulcerating skin lesions, a reduction of mediastinal adenopathies and/or a reduction of visceral metastases, in particular lung and/or liver metastases and/or results in a reduction of pain.
20. The anti-HER2 antibody according to any one of embodiments 1 to 19, having an amount of fucose in the CH2 domain of 20% or less, 15% or less, 10% or less, 5% or less, 10% to 3% or 0%, preferably in the range of from 2% to 20%, from 3% to 15% or from 5% to 10%.
21. The anti-HER2 antibody according to any one of embodiments 1 to 20, having an amount of fucose in the CH2 domain of 20% or less, preferably 15% or less, more preferred 10% or less and one or more, preferably all of the following glycosylation characteristics:
   (i) an amount of bisecting GlcNAc of at least 8%;
   (ii) an amount of galactose of at least 65%;
   (iii) optionally no detectable NeuGc;
   (iv) optionally no detectable Galα1,3-Gal;
   (v) optionally detectable α2,6-coupled NeuAc.
22. The anti-HER2 antibody according to any one of embodiments 1 to 21, having one or more, preferably at least two, more preferably all of the following characteristics:
   (i) it comprises a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3;
   (ii) it comprises a heavy chain variable region comprising the amino acid sequence of SEQ ID NO: 7 or an amino acid sequence which is at least 80% identical thereto;
   (iii) it comprises a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6;
   (iv) it comprises a light chain variable region comprising the amino acid sequence of SEQ ID NO: 8 or an amino acid sequence which is at least 80% identical thereto;
   (v) it shows cross-specificity with the antibody trastuzumab;
   (vi) it comprises heavy chain and light chain amino acid sequences which are at least 90% identical to the amino acid sequences of the antibody trastuzumab;
   (vii) it is equivalent to the antibody trastuzumab in binding and Fv mediated anti-tumor response;
   (viii) it was recombinantly produced in a human cell line.
23. The anti-HER2 antibody according to any one of embodiments 1 to 22, being capable of inducing a stronger ADCC than a corresponding high fucose anti-HER2 antibody.
24. The anti-HER2 antibody according to any one of embodiments 4 to 23, wherein the high fucose anti-HER2 antibody used in the preceding treatment has an amount of fucose in the CH2 domain of 70% or more.
25. The anti-HER2 antibody according to any one of embodiments 4 and 24, wherein the high fucose anti-HER2 antibody used in the preceding treatment has one or more, preferably at least three of the following characteristics:
   (i) it is an IgG antibody;
   (ii) it shows cross-specificity with the reduced fucose anti-HER2 antibody;
   (iii) it is capable of specifically binding to the same epitope as the reduced fucose anti-HER2 antibody;
   (iv) the amino acid sequences of its heavy chain variable region and light chain variable region are at least 80%, at least 90% or at least 95%, more preferably 100% identical to those of the reduced fucose anti-HER2 antibody;
   (v) it is the antibody trastuzumab (Herceptin®);
   (vi) it is capable of specifically binding to HER2, wherein the epitope of the high fucose anti-HER2 antibody is different from the epitope of the reduced fucose anti-HER2 antibody; and/or
   (vii) it is the antibody pertuzumab (Omnitarg).
26. The anti-HER2 antibody according to any one of embodiments 1 to 25, wherein the treatment with reduced fucose anti-HER2 antibody is a monotherapy.
27. The anti-HER2 antibody according to any one of embodiments 1 to 26, wherein the treatment with reduced fucose anti-HER2 antibody is a combination therapy, in particular in combination with
   (i) at least one chemotherapeutic agent; and/or
   (ii) at least one further therapeutic antibody which is different from the reduced fucose anti-HER2 antibody; and/or
   (iii) cancer surgery and/or radiotherapy.
28. The anti-HER2 antibody according to any one of embodiments 1 to 27, wherein
   a) the at least one chemotherapeutic agent used in the preceding treatment of the patient according to embodiment 4 is selected from the group consisting of cyclophosphamide; lapatinib; capecitabine; cytarabine; vinorelbine; bevacizumab; gemcitabine; maytansine; anthracyclines such as daunorubicin, doxorubicin, epirubicin, idarubicin, valrubicin and mitoxantrone; taxanes such as paclitaxel (Taxol), docetaxel (Taxotere) and SB-T-1214; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues; and/or
   b) wherein the treatment with reduced fucose anti-HER2 antibody is a combination therapy with at least one different anti-cancer agent, wherein the anti-cancer agent is selected from the group consisting of (i) chemotherapeutic agents, wherein the chemotherapeutic agent preferably is a taxane, and (ii) anti-cancer therapeutic antibodies, wherein the therapeutic antibody preferably is an anti-HER2 antibody which differs in its mode of action from the reduced fucose anti-HER2 antibody such as pertuzumab if the reduced fucose anti-HER2 antibody corresponds to trastuzumab, anti-EGFR antibodies such as cetuximab (Erbitux) and/or an anti-VEGF antibody such as bevacizumab (Avastin).
29. The anti-HER2 antibody according to any one of embodiments 1 to 28, wherein the preceding treatment of the patient involved the use of at least one therapeutic antibody different from the reduced fucose anti-HER2 antibody and which in particular is selected from the group consisting of anti-HER2 antibodies which differ in their mode of action from the reduced fucose anti-HER2 antibody, in particular pertuzumab, anti-EGFR antibodies such as cetuximab (Erbitux), panitumomab (Vectibix) and nimotuzumab (Theraloc); anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin).
30. The anti-HER2 antibody according to any one of embodiments 1 to 29, for administration of the reduced fucose anti-HER2 antibody in an amount of from 1 to 10 mg/kg body weight of the patient every first, second, third or fourth week or less frequently; preferably in an amount of from 2 to 5 mg/kg body weight of the patient every third week or less frequently.
31. The anti-HER2 antibody according to any one of embodiments 4 to 30, wherein the reduced fucose anti-HER2 antibody has a higher therapeutic efficacy than a corresponding high fucose anti-HER2 antibody when the reduced fucose anti-HER2 antibody is administered at the same dose but less frequently than the high fucose anti-HER2 antibody or when the reduced fucose anti-HER2 antibody is administered at the same frequency but at a lower dose than the high fucose anti-HER2 antibody.
32. The anti-HER2 antibody according to any one of embodiments 1 to 31, wherein the treatment of the anti-HER2 antibody is combined with a pre-medication of the patient with an agent with analgesic and/or antipyretic properties, in particular with N-(4-hydroxyphenyl)acetamide.
33. The anti-HER2 antibody according to embodiment 32, wherein the pre-medication comprises at least two separate doses of the agent with analgesic and/or antipyretic properties, whereas the first dose is given 8 h to 48 before the administration of the reduced fucose anti-HER2 antibody and the second dose is given 5 min to 6 hours before the administration of the reduced fucose anti-HER2 antibody.
34. The anti-HER2 antibody according to embodiment 33, wherein the each of the doses contains 250 mg and 1500 mg, in particular 1000 mg of the agent with analgesic and/or antipyretic properties.
35. The anti-HER2 antibody according to one of the embodiments 32 to 34, wherein the pre-medication further comprises the administration of a steroid, preferably a glucocorticoid, in particular methylprednisolone.
36. The anti-HER2 antibody according to embodiment 35, wherein the steroid is administered 5 min to 4 h, in particular 30 min before the administration of the reduced fucose anti-HER2 antibody.
37. The anti-HER2 antibody according to one of the embodiments 32 to 34, wherein the pre-medication comprises, or consists of, the following steps:
   a) a first dose of 1000 mg of N-(4-hydroxyphenyl) acetamide the evening before the administration of the reduced fucose anti-HER2 antibody,
   b) a second dose of 1000 mg of N-(4-hydroxyphenyl) 1 hour before the administration of the reduced fucose anti-HER2 antibody and
   c) one dose of 125 mg methylprednisolone 30 min before administration of the antibody.
38. An analgesic and/or antipyretic agent for treating or preventing infusion related reactions caused by the administration of a composition comprising reduced fucose anti-HER2 antibodies according to the pre-medication of any of the embodiments 32 to 37.

Specific and particularly preferred embodiments of this aspect will be described again in the following:

Specific and particularly preferred embodiments of the present invention will be described in the following:

In a first specific embodiment, the present invention is directed to a reduced fucose anti-HER2 antibody for treating a patient with a HER2 positive cancer, wherein the HER2 positive cancer has a HER2 overexpression of level 2+ or lower, preferably level 1+, as determined by immunohistochemistry (IHC) and wherein preferably, the cancer is a metastasizing cancer, wherein the reduced fucose anti-HER2 antibody
(i) has in the CH2 domain an amount of fucose of 20% or less, preferably 15% or less, more preferred 10% to 0% or 10% to 3% an amount of bisecting GlcNAc of at least 8% and an amount of galactose of at least 65%;
(ii) comprises a heavy chain variable region comprising the amino acid sequence of SEQ ID NO: 7 or an amino acid sequence which is at least 80% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3;
(iii) comprises a light chain variable region comprising the amino acid sequence of SEQ ID NO: 8 or an amino acid sequence which is at least 80% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6;
   and wherein prior to the treatment with the reduced fucose anti-HER2 antibody said patient has been treated with
a) at least one, at least two and preferably at least three different chemotherapeutic agents; and/or
b) at least one anti-HER2 antibody having an amount of fucose in the CH2 domain of 60% or more (high fucose anti-HER2 antibody), wherein the amino acid sequences of its heavy chain variable region and light chain variable region are at least 80%, preferably at least 90% identical to those of the reduced fucose anti-HER2 antibody, preferably trastuzumab (Herceptin®);
c) optionally radiotherapy; and
d) optionally at least one further therapeutic antibody;
   wherein the preceding treatments a), b), optionally c) and optionally d) occurred in any order sequentially or concurrently. Preferably, the preceding treatments in this first embodiment included one or more of the following
(i) at least one treatment with the high fucose anti-HER2 antibody trastuzumab (Herceptin®) as monotherapy and/or at least one combination treatment with a chemotherapeutic agent, preferably a taxane such as docetaxel and vinorelbine, in particular at least one monotherapy with the high fucose anti-HER2 antibody trastuzumab (Herceptin®) and additionally at least one, preferably at least two combination treatments with the high fucose anti-HER2 antibody trastuzumab (Herceptin);
(ii) at least one treatment with at least one taxane, preferably at least two separate treatments with one, two or more different taxanes, preferably with paclitaxel and docetaxel;
(iii) at least one treatment with a platinum based chemotherapeutic agent such as cisplatin, preferably in combination with a chemotherapeutic agent such as gemcitabine;
(iv) radiotherapy, preferably as adjuvant therapy;
(v) at least one, preferably at least two, at least three or at least four treatments with a chemotherapeutic agent or a combination of different chemotherapeutic agents such as a combination of doxorubicin and cyclophosphamide, a combination of lapatinib and capecitabine, a combination of idarubicine and etoposide and cytarabine, and a combination of bevacizumab and vinorelbine and capecitabine;
   and/or
(vi) surgical removal of at least a part of the primary tumor and/or one or more metastases.

In particular, the preceding treatments of the patient include in this first embodiment at least two, preferably at least three, at least four, at least 5 or all 6 of the treatments (i) to (vi). Preferably, the preceding treatments include at least treatments (i), (v) and (vi).

In a second specific embodiment, the present invention is directed to a reduced fucose anti-HER2 antibody for treating a patient with a HER2 positive cancer, wherein the HER2 positive cancer has a HER2 overexpression of level 2+ or lower, preferably level 1+, as determined by immunohistochemistry (IHC) and wherein preferably, the cancer is a metastasizing cancer, wherein the reduced fucose anti-HER2 antibody
(i) has in the CH2 domain an amount of fucose of 15% or less, preferably 10% to 0% or 10% to 2%, an amount of bisecting GlcNAc of at least 8% and an amount of galactose of at least 65%, has no detectable NeuGc, has no detectable Galα2,6-coupled NeuAc and preferably, the reduced fucose anti-HER2 antibody was recombinantly produced in a human cell line;
(ii) comprises a heavy chain variable region comprising the amino acid sequence of SEQ ID NO: 7 or an amino acid sequence which is at least 80% identical thereto, wherein the CDR1 has the amino acid sequence of SEQ ID NO: 1, the CDR2 has the amino acid sequence of SEQ ID NO: 2 and the CDR3 has the amino acid sequence of SEQ ID NO: 3;
(iii) comprises a light chain variable region comprising the amino acid sequence of SEQ ID NO: 8 or an amino acid sequence which is at least 80% identical thereto, wherein the CDR1 has the amino acid sequence of SEQ ID NO: 4, the CDR2 has the amino acid sequence of SEQ ID NO: 5 and the CDR3 has the amino acid sequence of SEQ ID NO: 6;
(iv) is capable of inducing a stronger ADCC than trastuzumab (Herceptin®);
   and wherein prior to the treatment with the reduced fucose anti-HER2 antibody said patient has been treated with
a) at least two, preferably at least three different chemotherapeutic agents; and
b) at least one anti-HER2 antibody having an amount of fucose in the CH2 domain of 60% or more (high fucose anti-HER2 antibody), wherein the amino acid sequences of its heavy chain variable region and light chain variable region are at least 80%, preferably at least 90% identical to those of the reduced fucose anti-HER2 antibody, preferably trastuzumab (Herceptin®);
c) optionally radiotherapy; and
d) optionally at least one further therapeutic antibody;
   wherein the preceding treatments a), b), optionally c) and optionally d) occurred in any order sequentially or concurrently and wherein the reduced fucose anti-HER2 antibody is for the treatment of a metastases selected from skin metastases, in particular ulcerating skin metastases, lymphnode metastases and visceral metastases, in particular lung or liver metastases. Preferred preceding treatments were described above in conjunction with the first specific embodiment, it is referred to the respective disclosure.

The patient that is treated in the first or second specific embodiment may have the following characteristics:
(i) the patient is homozygous for valine in amino acid position 158 of the Fcγ receptor IIIa (FcγRIIIa-158V/V); or
(ii) the patient is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F).

In particular, the reduced fucose anti-HER2 antibody of the first and second specific embodiment can be used for the treatment of patients irrespective of their FcγRIIIa allotype. In the first and second specific embodiment, the HER2 positive cancer and/or metastasis may have a HER2 overexpression of level 2+ or lower, preferably 1+ or lower, as determined by immunohistochemistry. Preferably, the HER2 positive cancer and/or metastasis is positive for HER2 gene amplification as determined by FISH or CISH. According to one aspect the patient to be treated in the first or second specific embodiment is homozygous for phenylalanine in amino acid position 158 of the Fcγ receptor IIIa (FcγRIIIa-158F/F) or the patient is heterozygous for valine and phenylalanine in amino acid position 158 of the Fcγ receptor IIIa (FcγRIIIa-158V/F) and optionally additionally the HER2 positive cancer and/or metastasis has a HER2 overexpression of level 2+ or lower, preferably 1+ or lower, as determined by immunohistochemistry.

Suitable and preferred dosages of the reduced fucose anti-HER2 antibody and suitable and preferred premedication schedules were described above; it is referred to the above disclosure which also applied to the first and second specific embodiment. The reduced fucose anti-HER2 antibody according to the first and second specific embodiment may be for use as monotherapy or as combination therapy. Embodiments were described above and it is referred to the respective disclosure.

As described above, the present invention is directed to a method of treatment of a human patient with a HER2 positive cancer, wherein the HER2 positive cancer has a HER2 overexpression of level 2+ or lower, preferably level 1+, as determined by immunohistochemistry (IHC) and wherein, preferably, the cancer is a metastasizing cancer, comprising administering an anti-HER2 antibody having an amount of fucose in the CH2 domain of 50% or less, preferably 30% or less, more preferably 15% to 0% (reduced fucose anti-HER2 antibody).

As described above, the present invention is directed to a method of treatment of a patient suffering from a HER2-positive neoplastic disease, in particular HER2 positive cancer after treatment with a high fucose anti-HER2 antibody, comprising administering a reduced fucose anti-HER2 antibody to said patient in an amount sufficient to treat the neoplastic disease. In particular, the reduced fucose anti-HER2 antibody has an amount of fucose in the CH2 domain of 50% or less and the high fucose anti-HER2 antibody has an amount of fucose in the CH2 domain of 60% or more. In preferred embodiments, prior to the treatment with the reduced fucose anti-HER2 antibody said patient has been treated with
a) at least one chemotherapeutic agent;
b) at least one anti-HER2 antibody having an amount of fucose in the CH2 domain of fucose 60% or more (high fucose anti-HER2 antibody);
c) optionally radiotherapy; and
d) optionally at least one further therapeutic antibody;
   wherein the preceding treatments a), b), c) and d) occurred in any order sequentially or concurrently. The cancer can be a HER2 positive cancer has a HER2 overexpression of level 2+ or lower, preferably level 1+, as determined by immunohistochemistry (IHC),

All the embodiments and features described above also likewise apply to the methods of treatment according to the invention.

The present application claims the benefit of prior applications US 61/673,201, filed on Jul. 18, 2012, US 61/673,216, filed on Jul. 18, 2012, US 61/673,229, filed on Jul. 18, 2012, and EP 12 197 768.0, filed on Dec. 18, 2012, which are all incorporated herein by reference.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### FIGURES

FIG. 1 shows the serum half-life t1/2 as a function of the infusion dose/body weight after the first infusion. Fuc− trastuzumab: black circles; Fuc+ trastuzumab (Herceptin®): gray circles.

FIG. 2 shows the dramatic healing of ulcerating skin metastases which started within 8 days after the 1′ dose and was complete after 6 weeks (2^{nd} dose). Dosage: 240 mg Fuc− trastuzumab every third week. A: Before treatment (baseline); B: After 1 week (cycle 1, day 8), 1 dose of 240 mg Fuc− trastuzumab; C: After 3 weeks (cycle 2, day 1), 1 dose of 240 mg Fuc− trastuzumab; D: After 5 weeks (cycle 2, day 15), 2 doses of 240 mg Fuc− trastuzumab; E: After 6 weeks (cycle 3, day 1), 2 doses of 240 mg Fuc− trastuzumab.

FIG. 3 shows binding of Fuc− trastuzumab and Fuc+ trastuzumab (Herceptin®) on different cell lines analyzed by flow cytometry. Mean values of duplicates ±SD are shown. A: Herceptin® B: Fuc− trastuzumab.

FIG. 4A shows HER2/neu expression on ZR-75-1 cells after 4 days of incubation with Fuc− trastuzumab and Fuc+ trastuzumab (Herceptin®). Mean values of the percentage of HER2 positive cells relative to the medium control ±SD obtained from two independent flow cytometry experiments each performed in duplicates are shown.

FIG. 4B shows a Western blot of lysates from ZR-75-1 cells incubated with Fuc− trastuzumab, Fuc+ trastuzumab (Herceptin®) or hlgG1 and medium as a negative control at a concentration of 0.1 μg/ml for 3 days.

FIG. 5 shows Proliferation inhibition of SK-BR-3 cells by Fuc− trastuzumab and Fuc+ trastuzumab (Herceptin®). Incubation time with the antibodies was 4 days. Percentage of proliferation compared to the medium control is shown. Mean values+SEM of 3 independent experiments performed with 6 replicate measurements are given.

FIG. 6 shows an active caspase-3 apoptosis assay using BT474 cells after 6 h incubation with Fuc− trastuzumab and Fuc+ trastuzumab (Herceptin®) and protein G. Mean values of the percentage of cleaved caspase-3 positive cells (apoptotic cells) ±SD of measurements in duplicates are shown.

FIG. 7 shows an ADCC assay on SK-BR-3 cells with Fuc− trastuzumab and Fuc+ trastuzumab (Herceptin®) using primary human PBMC of donors with different FcγRIIIa allotypes. Mean values of specific lysis ±SEM of triplicates are given. A: VV donor; B: FV donor; C: FF donor.

FIG. 8 shows an ADCC assay on MCF-7 cells with primary human PBMC of donors with different FcγRIIIa allotypes. Incubation time 5 h, E:T ratio 50:1. Mean values of specific lysis ±SEM of triplicates are given. A: VV donor; B: FV donor; C: FF donor.

FIG. 9 (A and B) shows a comparison of the concentrations of Fuc− trastuzumab and Herceptin® that are required in order to achieve the same specific lysis on MCF-7 cells (at a specific lysis of 95% of the maximal lysis of Herceptin) as well as the factor (improvement factor) by which the concentration of Fuc− trastuzumab was reduced in order to achieve the same specific lysis as Herceptin® at 95% of its maximal lysis. Black symbols represent individual donors, red symbols mean values of all donors (see A). Mean values of all donors are shown as lines in B. The maximum Fuc− trastuzumab mediated ADCC increase for lower HER2 expressing tumors (MCF-7) is up to 140 fold, with 42 in the mean. Thus, a largely improved anti-tumor ADCC was achieved for all patient allotypes.

FIGS. 10, 11 and 12 show results of experiments similar to the ones shown in FIGS. 6 to 8. Further explanations are provided in the description of the corresponding example 15.

FIG. 13A shows the in vivo antitumor activity of Fuc− trastuzumab and Herceptin® in nude mice bearing the BT474 human breast carcinoma xenograft. Mice were treated at the indicated dosage level when tumors reached palpable size. The antibodies were administered i.v. twice weekly for 4 weeks. Each symbol represents the mean value and SEM of a group of 8 animals. FIG. 13B shows the in vivo antitumor activity of Fuc− trastuzumab at different concentrations in nude mice bearing the BT474 human breast carcinoma xenograft. Mice were treated at the indicated dosage level when tumors reached palpable size. The antibodies were administered i.v. twice weekly for 4 weeks. Each symbol represents the mean value and SEM of a group of 8 animals.

FIG. 14 shows the in vivo anti-tumor activity of Fuc− trastuzumab in nude mice bearing the patient derived #7268 gastric carcinoma xenografts (MV9138). Mice were treated at the indicated dosage level when tumors reached palpable size. The antibodies were administered i.v. twice weekly for 4 weeks. Each symbol represents the mean value and SEM of a group of 8 animals.

FIG. 15A shows the pharmacokinetics of Fuc− trastuzumab and Herceptin® following a single dose i.v. administration of 30 mg/kg body weight. The antibody serum concentration of the animals was measured at 10 time points post dosage. Each symbol represents the mean value and SEM of a group of 3 animals. Data points were fitted with a two phase exponential decay weighted with 1/Y². FIG. 15B shows the serum concentrations of Fuc− trastuzumab and Herceptin® after a single i.v. infusion. Each symbol represents the mean value and standard deviation of a group of 3 m animals.

### EXAMPLES

### Example 1

### Glycosylation Analysis of Trastuzumab Variants

A reduced fucose anti-HER2 antibody according to the present invention, here a low fucosylation variant of trastuzumab (Fuc− trastuzumab, also referred to subsequently as TrasGEX™) was obtained by expression in a human myeloid leukemia cell line having a reduced fucosylation activity as described in WO 2008/028686 A2, herein incorporated by reference. The high fucose anti-HER2 antibody trastuzumab (Fuc+ trastuzumab) was produced in hamster CHO cells and thus substantially corresponds to trastuzumab (Herceptin®).

To characterize the glycosylation pattern of the Fuc− trastuzumab in more detail, glycoprofiling studies were performed. The humanized IgG1 antibody trastuzumab comprises one N-glycosylation site in the heavy chain constant region 2. For glycoprofiling, the intact N-glycans were released from the protein core and the reducing ends of N-glycans were labeled with a fluorescence marker. The purified sample of the labeled N-glycans was separated by UPLC. Peak areas based on fluorometric detection were employed for calculation of the relative molar abundances of the N-glycan structures. Estimated data for the antibody are summarized in Table 1. The values represent the relative molar contents of N-glycans containing the type of monosaccharide of interest (e.g. fucose).

TABLE 1

| | Rel. abundance [mol %]* | | | | |
|---|---|---|---|---|---|
| Sample | F | S > 0 | G > 0 | G2 | B |
| Fuc⁻ trastuzumab | 8 | 7 | 73 | 25 | 12 |
| Fuc⁺ trastuzumab | 86 | 1 | 42 | 6 | 0 |

*Relative abundances of glycan structures are related to the total amount of N-glycans.
F = fucosylated N-glycans;
S > 0 = sialylated N-glycans;
G > 0 = galactosylated N-glycans;
G2 = N-glycans with two galactoses;
B = N-glycans with bisecting N-acetylglucosamine.

The glycoprofiling shows that Fuc− trastuzumab has a much lower fucose content and a higher bisGlcNAc content compared to the Fuc+ trastuzumab expressed in hamster CHO cells (as are used for the production of Herceptin®). Furthermore, the Fuc− trastuzumab had due to the production in a human cell line a human glycosylation profile and thus no detectable NeuGc and no detectable Galα1,3-Gal.

Target binding, specificity, affinity and Fv mediated anti-tumor activity of the Fuc− trastuzumab and the Fuc+ trastuzumab (Herceptin®) were analyzed in different comparability studies (see also examples below), in particular HER2 antigen ELISA, flow cytometry analysis, HER2 downmodulation, reduction of VEGF production, inhibition of tumor proliferation and the induction of tumor apoptosis. The results confirmed that the Fuc− trastuzumab according to the present invention shows full maintenance of tumor cell proliferation inhibition and induction of tumor cell apoptosis. Therefore, the Fuc− trastuzumab and the Fuc+ trastuzumab are basically equivalent in binding and Fv mediated anti-tumor properties. Thus, the improvements regarding the therapeutic efficacy and in particular the anti-metastatic activity are attributable to the improved glycosylation characteristics of the reduced fucose anti HER2 antibody.

The Fuc− trastuzumab as described in example 1 was used in the subsequent analyses and examples.

### Example 2

### Clinical Studies

A phase I-dose escalation and pharmacokinetic study of Fuc− trastuzumab (see example 1) in patients with locally advanced or metastatic HER2-positive cancer was performed. A three-weekly dosing scheme was used. The patients received either 12 mg, 60 mg, 120 mg, 240 mg, 480 mg or 720 mg of the Fuc− trastuzumab. The treatment was safe and very well tolerated with only occasional infusion-related reactions (IRR) mainly at first infusion which can be controlled by steroids, and in particular with a combination of paracetamol and steroids as described herein.

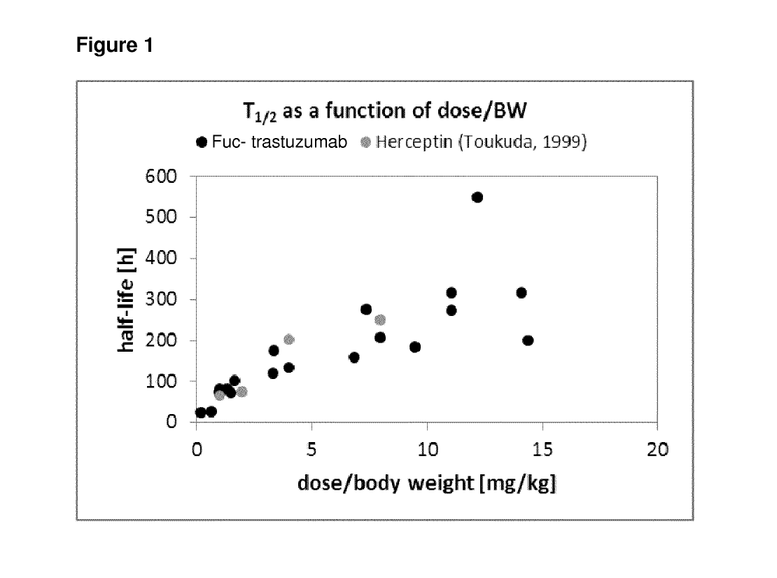

Regarding the observed pharmacokinetics, the Fuc− trastuzumab showed fully comparable pharmacokinetic properties to Herceptin® including the serum half-life, Cₘₐₓ, Cₘᵢₙ, AUC and clearance. For example, the circulation half-life t_{1/2} of the Fuc− trastuzumab after the first infusion was dose dependent and fully comparable to Herceptin®, with serum t_{1/2} after 480 mg infusion being 213±59 h and serum t_{1/2} after 720 mg infusion being 306±131 h (see FIG. 1)

An impressive therapeutic efficacy was seen in these late stage patients which received multiple prior treatments of chemotherapy and/or antibody therapy. A therapeutic effect was seen in patients which did not respond previously to Herceptin®. Furthermore, responses were seen at doses lower than those used for Herceptin®. Furthermore, the therapeutic efficacy was also seen in patients with a low HER2 expression such as 1+ and 2+(determined by IHC), wherein a stabilization of the disease over months could be achieved.

TABLE 2
HER2 status of the patients treated with Fuc− trastuzumab
in the clinical study.

| HER2 status | 1+ | 2+ | 3+ |
|---|---|---|---|
| percentage of total patients | 36% | 19% | 44% |
| patients with the respective HER2 status | 31% | 43% | 50% |
| showning at least stable disease | | | |
| average no. of treatment cycles of the | 5.3 | 5.7 | 8.3 |
| patients with at least stable disease | | | |
| average time to progression of the patients | 106 | 108 | 154 |
| with at least stable disease [days] | | | |

The Fuc− trastuzumab shows comparable therapeutic efficacy in the patients with different FcγRIIIa status, demonstrating that the treatment with the Fuc− trastuzumab is independent of the FcγRIIIa allotype:

TABLE 3
FcγRIIIa status of the patients treated with Fuc− trastuzumab
in the clinical study.

| FcγRIIIa 158 allytype | F/F | F/V | V/V |
|---|---|---|---|
| percentage of total patients | 39% | 44% | 17% |
| patients with the respective FcγRIIIa status | 50% | 38% | 50% |
| showning at least stable disease | | | |
| average no. of treatment cycles of the | 5.9 | 7.8 | 6.7 |
| patients with at least stable disease | | | |
| average time to progression of the patients | 99 | 160 | 130 |
| with at least stable disease [days] | | | |

Furthermore, a therapeutic efficacy was seen in indications where Herceptin® does not show a significant therapeutic effect. In particular, a high efficacy was seen on metastases, in particular skin metastasis, in particular ulcerating skin metastasis, lung and liver metastasis, lymph node lesions and furthermore, also a reduction of pain was observed which significantly improves the quality of life for patients, in particular for incurable patients. Furthermore, effective treatment of patients having colon cancer, non-small cell lung carcinoma (NSCLC), bronchial cancer or parotid gland carcinoma, respectively, was observed. Therefore, the clinical data obtained confirm the high therapeutic efficacy of the reduced fucose anti-HER2 antibodies according to the present invention in the novel treatment schedules and patient groups described herein.

Selected records of patients who had a major response upon administration of the reduced fucose anti-HER2 antibodies according to the present invention, here Fuc− trastuzumab (see example 1) are described in the following examples. Tumor evaluation was performed during the clinical study according to the guidelines for Response Evaluation Criteria in Solid Tumors (RECIST) published by an international collaboration including the European Organisation for Research and Treatment of Cancer (EORTC), the National Cancer Institute of the United States, and the National Cancer Institute of Canada Clinical Trials Group

### Example 3

### Treatment of a Heavily Pretreated Patient Afflicted with Metastatic Breast Cancer with Fuc− Trastuzumab

### Patient Characteristics: Female, F/F FcγIIIa Status

### Chronology of the Pretreatment:

### September 2006:

diagnosis of right locally advanced breast cancer (histology: invasive ductal carcinoma; ER− PgR−; Herceptest 3+).

### From September 2006 to January 2007:

4 cycles of therapy with doxorubicin and cyclophosphamide followed by 2 cycles of therapy with paclitaxel, with partial response of disease.

### February 2007:

right mastectomy (histology: invasive ductal carcinoma GIII; ER− PgR−; Herceptest 3+).

### From March to June 2007:

3 cycles of therapy with paclitaxel, followed by radiotherapy on right chest wall, right axillary and supraclavicular region and right internal mammary chain.

### From April 2007 to June 2008:

treatment with trastuzumab (Herceptin® Fuc+).

### March 2009:

recurrence of disease (skin metastases at chest wall, mediastinal adenopathies). A biopsy of skin metastases was performed that confirmed the localization of breast cancer (ER−; PgR−; HER2/neu 3+).

### From April to May 2009:

3 cycles of therapy with trastuzumab (Herceptin®) and docetaxel, with progression of disease.

### From June 2009 to January 2010:

treatment with lapatinib and capecitabine, with initial partial response of disease, followed by progression of disease.

### From February to September 2010:

treatment with idarubicine, etoposid and cytarabine, with initial partial response of disease, followed by progression of disease.

### October 2010:

a biopsy of skin metastases was repeated (histology: localization of breast cancer; ER−; PgR−; HER2/neu 3+).

### From December 2010 to February 2011:

treatment with trastuzumab (Herceptin®) and vinorelbine, with progression of disease.

### From March to August 2011:

8 cycles of therapy with bevacizumab, vinorelbine and capecitabin, with initial partial response of disease, followed by progression of disease.

### From October 2011 to February 2012:

4 cycles of therapy with cisplatin and gemcitabine, with progression of disease.
   Treatment with Fuc− Trastuzumab

After the failed pretreatments described above, the patient was enrolled into a study with Fuc− trastuzumab in March 2012. At baseline the patient was rapidly progressing with skin lesions and medistinal lymph node infestation. In particular, there was a diffuse cutaneous neoplastic involvement at chest wall with multiple bleeding areas of skin ulceration. The area of skin ulceration was larger in right parasternal region (maximum diameter 6 cm). The CT scan confirmed the presence of mediastinal adenopathies. As can be derived from the above described pretreatments, no effect was seen with Herceptin® in one monotherapy and two combination therapies.

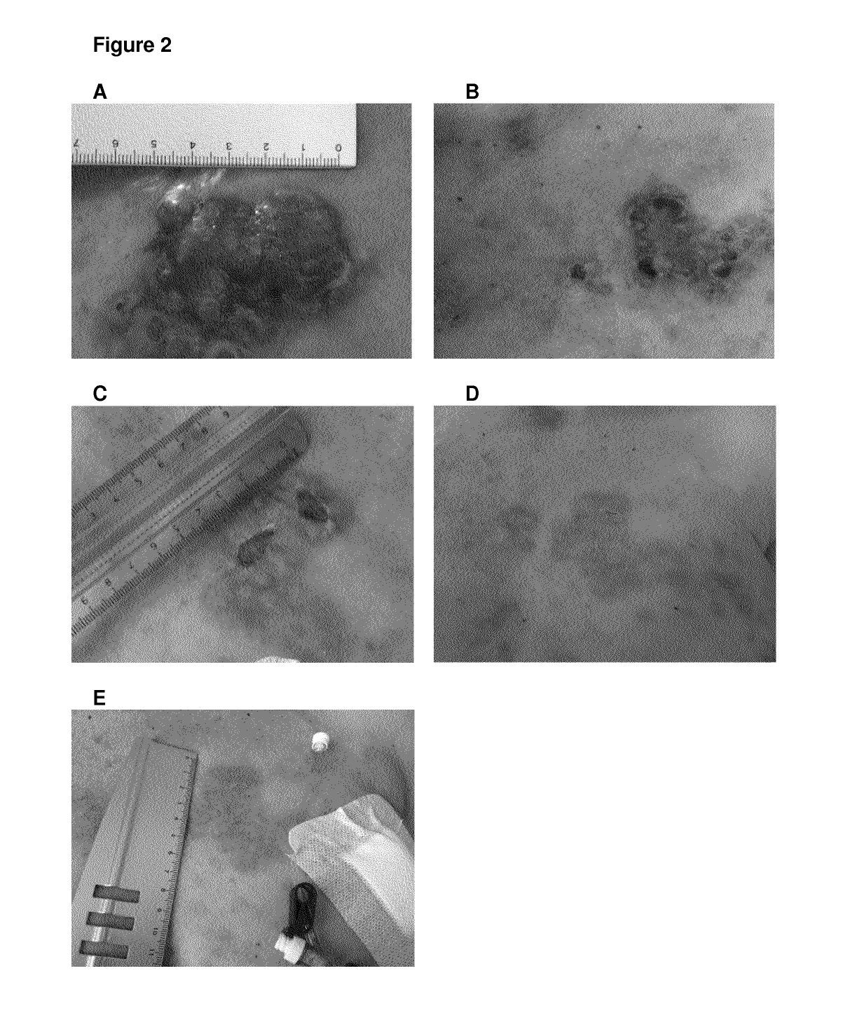

The patient received 6 cycles of therapy with Fuc− trastuzumab (240 mg every third week, which translates into a dosage of approx. 3.3 mg/kg), well tolerated. A reparation process of the skin ulceration area was already noticeable at day 8 of cycle 1, i.e. after the initial dose, and became gradually more. After cycle 3 a partial response of disease has been documented. The cutaneous neoplastic infiltration is globally improved; particularly the area of skin ulceration in right parasternal region was fully repaired (see FIG. 2). Furthermore, a strong reduction of lymph node infestation (mediastinal adenopathies) was reported. 72% reduction of target sum at first CT scan and 3 cycles of Fuc− trastuzumab was reported (sum of the longest diameters: reduction from 135 mm to 37 mm).

Example 3 demonstrates the high efficacy of reduced fucose anti-HER2 antibodies in heavily pretreated patients that failed treatment with a high fucose anti-HER2 antibody (Herceptin®) and numerous chemotherapeutic treatments and in particular showed a remarkable effect on ulcerating skin metastases and lymph node metastases. Example 3 thus supports the important contribution the present invention makes to the prior art.

### Example 4

### Treatment of a Heavily Pretreated Patient Afflicted with Colon Cancer with Fuc− Trastuzumab

### Patient Characteristics: Female, FcγIIIa Status: F/V

### Characteristics of the Initial Cancer:

metastatic colon cancer stage IV
invasive sigmarectum carcinoma with liver and lung metatstases
HER2 3+ (Herceptest; complete membrane staining in 95% of cells)

### Pretreatment:

8 lines of prior treatments:
various chemotherapies comprising the chemotherapeutic agents Folfox, Folfiri, tegafur uracil and calcium folinate I
including combinations with anti-cancer antibodies: panitumomab (anti-EGFR monoclonal antibody), 4*Avastin=bevacizumab (anti-VEGF monoclonal antibody)
   at base line progressing with lung and liver metatstases.
   Treatment with Fuc− Trastuzumab:

The patient received 5 cycles of therapy with Fuc− trastuzumab (480 mg every three weeks, which translates into a dosage of approx. 7.5 mg/kg). The treatment was well tolerated and important therapeutic effects were documented. In particular, a 44% reduction of target lesions (sum of the longest diameters: reduction from 197 mm to 111 mm) was achieved (documented at first CT scan after 8 weeks). Example 4 demonstrates that the treatment with the reduced fucose anti-HER2 antibody according to the present invention is surprisingly effective in treating visceral metastases, such as in particular lung and liver metastases. This is an important finding because the prior art describes that high fucose anti-HER2 antibodies such as trastuzumab (Herceptin®) are not effective on lung and liver metastases. As described in the background of the invention, visceral metastases such as lung and liver metastases are the dominant side of relapse, in particular in patients that were treated with high fucose anti-HER2 antibodies such as trastuzumab (Herceptin®). These important findings described in this application provide novel treatment options for patients afflicted with visceral metastases, in particular lung and liver metastases as such metastases can be treated with the reduced fucose anti-HER 2 antibody according to the present invention.

### Example 5

### Treatment of a Patient Afflicted with Lung Cancer with Fuc− Trastuzumab

### Patient Characteristics: Male, FcγIIIa Status: F/F

### Characteristics of the Initial Cancer:

Non-small cell lung carcinoma (NSCLC) stage IV
HER2 3+
TNM cancer staging: CT1BN0M1A (tumor with 2 to 3 cm diameter; no spread to lymph nodes; near metastases in lung or pleural or pericardial fluid)

### Chronology:

Thyroidectomy in 1997 due to thyroid carcinoma.
Autoimmune hemolytic anemia in January 2013. Diagnosis of Non Small Cell Lung Carcinoma
   Treatment with Fuc− Trastuzumab:

The patient was enrolled into a study with Fuc− trastuzumab in February 2013. At baseline two lesions were identified as target lesions. To date the patient has received 6 cycles of therapy with Fuc− trastuzumab (720 mg every third week, which translates into a dosage of approximately 9.5 mg/kg). The sum of diameters of the target lesions remained unchanged between baseline and assessment after two months at which the tumor response evaluated according to RECIST 1.1. was Stable Disease. Treatment is currently ongoing.

### Example 6

### Treatment of a Patient Afflicted with Parotid Gland Cancer with Fuc− Trastuzumab

### Patient Characteristics: Male, FcγIIIa Status: F/F

### Characteristics of the Initial Cancer:

Right parotid gland carcinoma stage IIB
HER2 3+
TNM cancer staging: CT3CN0CM0 (tumor with more than 7 cm diameter; no spread to lymph nodes; no metastases)

### Chronology:

Initial diagnosis in December 2011, enoral tumor extirpation
Local relapse in January 2013
Adjuvant radiotherapy of retromandibular fossa at lymph node level I-Ill in January to March 2013
   Treatment with Fuc− Trastuzumab:

The patient was enrolled into a study with Fuc− trastuzumab in February 2013. At baseline one lesion was identified as target lesion. To date the patient has received 6 cycles of therapy with Fuc− trastuzumab (720 mg every third week, which translates into a dosage of approximately 8.9 mg/kg). The sum of diameters of the target lesion decreased by 26% between baseline and assessment after two months. The tumor response evaluated according to RECIST 1.1. was Stable Disease. Treatment is currently ongoing.

### Example 7

### Treatment of a Patient Afflicted with Bronchial Cancer with Fuc− Trastuzumab

### Patient Characteristics: Female, FcγIIIa Status: F/F

### Characteristics of the Initial Cancer:

bronchial cancer stage IIIB
HER2 2+
TNM cancer staging: T2N3M0 (tumor with 3 to 7 cm diameter; spread to distant lymph nodes; no metastases)

### Chronology:

Initial diagnosis in June 2012, cervical lymph node biopsy
13 cycles of therapy with vinorelbine from July 2012 to January 2013
   Treatment with Fuc− Trastuzumab:

The patient was enrolled into a study with Fuc− trastuzumab in February 2013. At baseline one lesion was identified as target lesion. To date the patient has received 6 cycles of therapy with Fuc− trastuzumab (720 mg every third week, which translates into a dosage of approximately 12.2 mg/kg). After two months the tumor response evaluated according to RECIST 1.1. was Stable Disease. Treatment is currently ongoing.

### Example 8

### Treatment of a Heavily Pretreated Patients Afflicted with HER2 Positive Cancers that Moderately Express HER2 with Fuc− Trastuzumab

Furthermore, in the performed clinical studies, a strong therapeutic effect was seen in patients afflicted with different HER2-positive cancers, which show an HER2 overexpression of only 1+ or 2+(as determined by IHC):

One patient (female, FcγIIIa status: F/V, HER2 expression 1+ as determined by IHC) afflicted with urothelial carcinoma (stage IV) also showed a major clinic response upon study enrollment, despite the low HER2 expression status of the cancer. The urothelial carcinoma was a bladder with perithoneal carcinomatosis and retroperithoneal lymphoadenopaties. The patient was previously treated with several chemotherapeutic agents, including carboplatin and gemcitabine. Furthermore, the patient received several cancer surgical procedures. The tumor reoccurred three times. Thus, despite the performed surgeries and despite the chemotherapeutic treatments, the patient was afflicted with target lesions and showed metastases upon study enrollment. In this patient, a stabilization of the disease was achieved when administering the Fuc− trastuzumab in a dosage of only 240 mg per infusion, which translates into a dosage of approximately 3.5 mg/kg. Furthermore, the patient reported a strong reduction of pain which is also an important clinical effect. These results confirm that the reduced fucose anti-HER2 antibodies according to the present invention allow the treatment of HER2 positive neoplastic diseases, which only show a moderate overexpression of HER2 and even of only 1+ as determined by IHC.

Similar results were also observed in a patient afflicted with progressing mammacarcinoma (female, FcγIIIa status: F/F, HER2 expression 1+ as determined by IHC). Also here, a stabilization of the disease was observed even when administering a very low dosage of 60 mg Fuc− trastuzumab what translates into a dosage of approx. 1 mg/kg (this patient was enrolled in a cohort which received the lower antibody dosages).

A further patient afflicted with invasive ductal mammacarcinoma (female, FcγIIIa status: F/F, HER2 expression 2+ as determined by IHC) also showed an ongoing stabilization upon receipt of Fuc− trastuzumab, even though the antibody was administered at a very low dosage of 60 mg in each cycle (this translates again into a concentration of approx. 1 mg/kg). Considering the responses that were seen at the higher dosages, it is evident that a stronger response will be visible upon administration of a higher dosage also in respective HER2 positive cancers that are characterized by a low HER2 expression.

### Example 9

### Adverse Reactions

In the clinical studies performed, also the adverse reactions caused by the treatment with the Fuc− trastuzumab were observed. During the study, no cardiac symptoms were detected. This is important as Herceptin is known to cause cardiac reactions. Furthermore, due to the heavy pretreatment and the far progressed disease status of the enrolled patients, said patients were generally of poor health and hence are more susceptible to cardiac diseases.

Furthermore, the Fuc− trastuzumab was also generally well tolerated and showed only mild or moderate adverse events. In particular, no adverse gastrointestinal reactions such as diarrhea, nausea and vomiting were observed and the patients had no adverse alterations in their blood count.

### Example 10

### Prevention of Infusion Related Reactions

In the clinical study for assessing the therapeutic activity of Fuc− trastuzumab, also mild to moderate adverse reactions caused by infusion of the Fuc− trastuzumab (infusion related reactions, IRR) were observed in patients of the first and second cohort. To prevent IRR in the subsequent administrations of Fuc− trastuzumab, the remaining patients were pretreated with paracetamol either alone or in combination with the steroid methylprednisolone prior to the Fuc− trastuzumab infusion. Paracetamol pretreatment involved one dose of 1000 mg the evening before and one dose of 1000 mg 1 h before the Fuc− trastuzumab infusion. Methylprednisolone was additionally administered to some patients 30 min before the Fuc− trastuzumab infusion at a dose of 125 mg. The pretreatment of the patients resulted in a decrease in IRR. In particular, nearly 50% of the patients who received the pretreatment with paracetamol and optionally methylprednisolone did not show any IRR upon Fuc− trastuzumab infusion. This was even more remarkable since the first patients who were not pretreated and showed IRR received the lowest doses of the Fuc− trastuzumab. Thus, the patients were IRR could be completely prevented by pretreatment with paracetamol and optionally methylprednisolone received up to 40-times higher Fuc− trastuzumab doses.

After introduction of pre-medication of steroids and paracetamol, only one of seven patients showed an IRR. Said patient showed IRR grade 2 which were restricted to the first and second infusion. Without the use of steroids, IRRs occurred in all but one patient, at least once (grade 1 to 3). Therefore, it is recommended to use a pre-medication comprising and preferably consisting of steroids and paracetamol and to restrict the respective pre-medication to the first infusion and the single next infusion following an IRR. Preferably, paracetamol is given on the evening before and one hour before the infusion with the reduced glucose anti-HER2 antibody. Steroids (for example 125 mg methylprednisolone) preferably will be administered 30 minutes before the infusion to the patient. In the absence of any IRR (IRR≧grade 1) at the first infusion, no pre-medication is needed to be given for the following infusions.

In case any IRR (IRR≧grade 1) occurs during the first or one of the following infusions, premedication (for example paracetamol and steroids as described above) will be administered to the patient for the next infusion and as long as an IRR (IRR≧grade 1) has been observed during the last infusion. In case the patient is experiencing second relapse of any IRR (IRR≧grade 1), it is recommended that this patient will receive premedication (paracetamol and steroids) for all subsequent infusions.

These findings demonstrate that IRR caused by Fuc− trastuzumab infusion can be prevented by pretreatment with an analgesic agent such as paracetamol, optionally in combination with a steroid such as methylprednisolone.

### Example 11

### Binding of the Differently Fucosylated Antibody Variants to Different Cells Expressing HER2

Several HER2 positive cell lines were analyzed by flow cytometry in order to compare the binding properties of Fuc− trastuzumab and Fuc+ trastuzumab (Herceptin®).

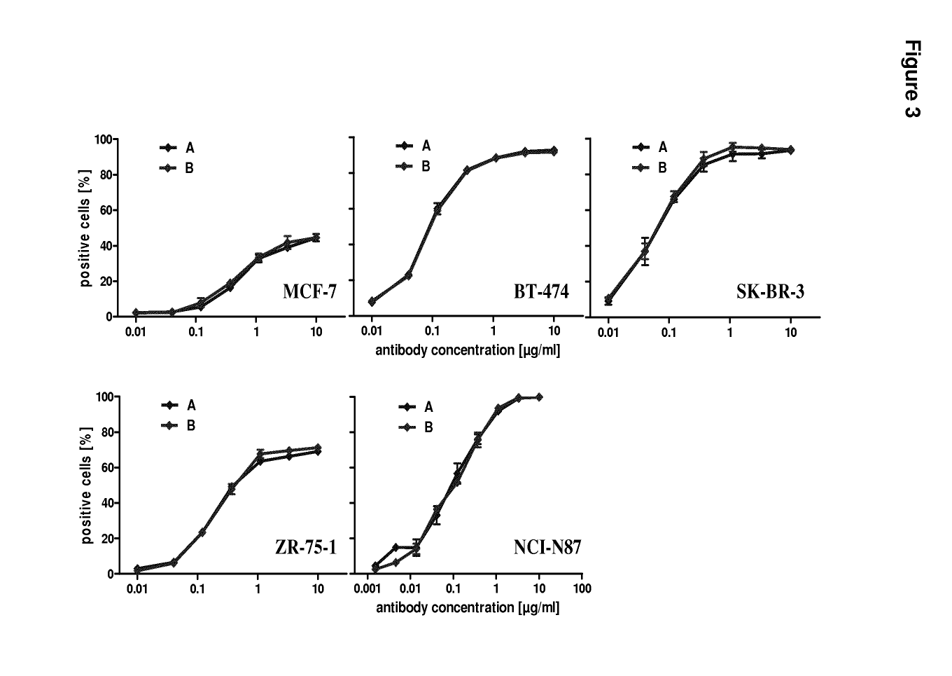

Briefly, target cells were harvested and incubated with the trastuzumab variant at different concentrations. Cells were washed and incubated with a secondary Cy3-conjugated anti-human IgG antibody at 4° C. in the dark. Cells were washed and analyzed in a flow cytometer FACS Canto II (Becton Dickinson). Live cells were gated based on their scatter properties and the percentage of positive cells was calculated using the FACSDiva Software (Becton Dickinson). As result, Fuc− trastuzumab and Fuc+ trastuzumab (Herceptin®) show comparable binding characteristics on all tumor cell lines tested as shown in FIG. 3. There were no differences in the percentage of positive cells over the whole analyzed concentration range of 0.01 to 10 μg/ml.

### Example 12

### Down-Modulation of the HER2 Receptor by Reduced and High Fucose Antibody Variants

Due to the identical binding specificity and affinity and its protein sequence of the variable region, it was expected that mechanisms mediated by binding of the antibody to the HER2 receptor without further Fc portion mediated interactions would be identical for Fuc− and Fuc+ trastuzumab. Binding of Herceptin® to the HER2 receptor is reported to down-modulate the expression of the receptor on the cell surface (Murphy et al., 2009; Cuello et al., 2001; Frankel, 2002; De Lorenzo et al., 2007). Reduced HER2 receptor expression on the cell surface allows less HER2 heterodimer formation, resulting in inhibition of growth factor-induced signaling, cell cycle progression and proliferation.

In order to analyze the capacity of Fuc− trastuzumab to down-modulate the HER2 receptor expression in a similar way as Herceptin®, a receptor down-modulation study was performed comparing this mechanism of action for both antibodies. HER2 receptor down-modulation was analyzed by flow cytometry and Western blot.

For flow cytometry analyses, ZR-75-1 cells were seeded into 96 well flat bottom plates and incubated for one day at 37° C. in a CO₂ incubator. Fuc− trastuzumab, Herceptin® or hlgG1 as a negative control at different concentrations were added. The plates were incubated for 3 to 4 days at 37° C. in a CO₂ incubator. ZR-75-1 cells were harvested and stained with a FITC-conjugated anti-human HER2 antibody (BMS120FI, eBioscience, Bender Medsystems) recognizing an epitope different from that bound by trastuzumab. Using this antibody, staining of the HER2 receptor is possible despite the presence of trastuzumab. BMS120FI positive cells were analyzed by flow cytometry at a BD FACS Canto II flow cytometer using BD FACSDiva Software.

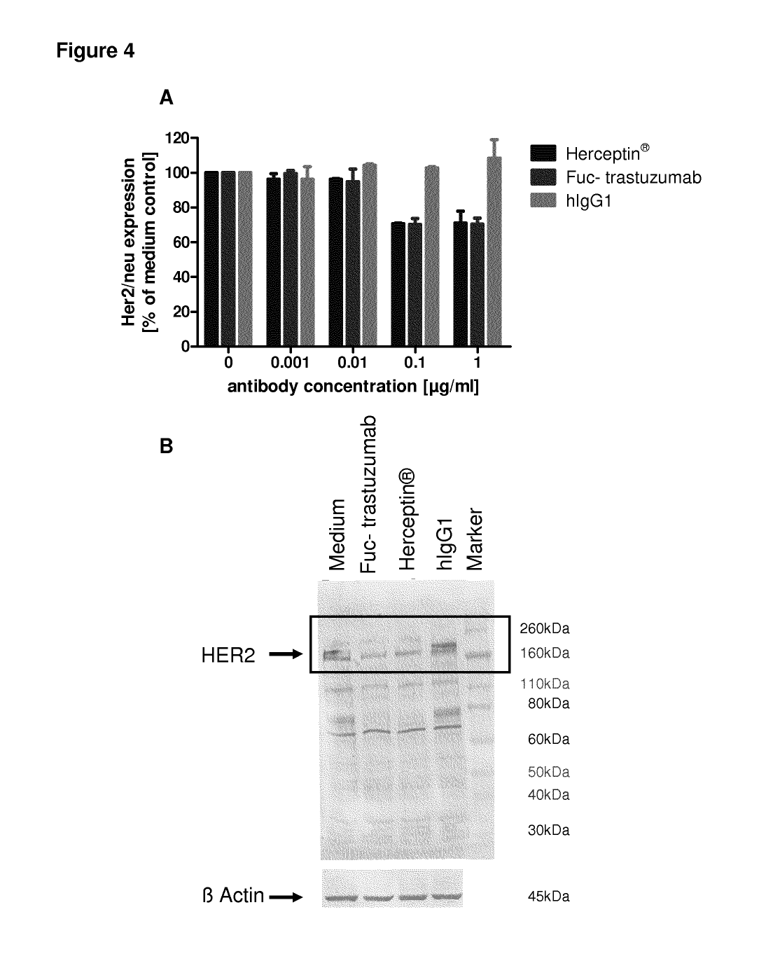

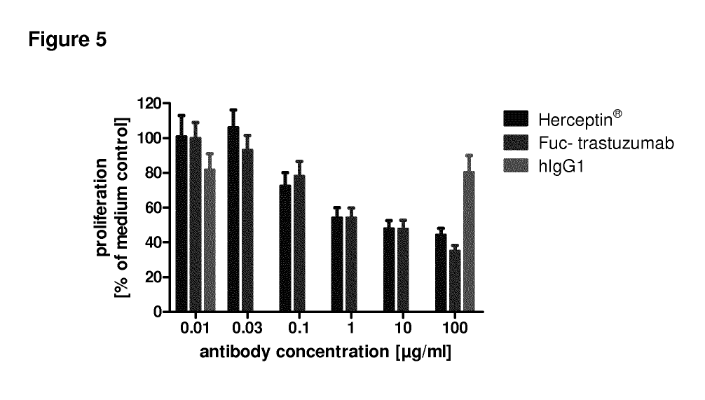

FIG. 4A shows the mean results of two independent assays using ZR-75-1 cells after 4 days of incubation with Fuc− trastuzumab, Herceptin® or hlgG1. Data represent HER2 receptor expression as a percentage of the medium control. It could be shown that the HER2 expression in the presence of Fuc− trastuzumab or Herceptin® was reduced by about 30% compared to the medium control. The human IgG1 isotype control did not result in a reduced HER2 receptor expression.

Therefore, binding of Fuc− trastuzumab to ZR-75-1 cells induces the down-regulation of the HER2 receptor on the cell surface. The level of HER2 down-regulation was comparable between Fuc− trastuzumab and Fuc+ trastuzumab (Herceptin®).

The results of the flow cytometric analyses were confirmed by Western blot. Briefly, ZR-75-1 cells were seeded into 10 cm cell culture dishes and incubated for one day at 37° C. in a CO₂ incubator. Fuc− trastuzumab, Herceptin® or hlgG1 as a negative control at a concentration of 0.1 μg/ml were added. The plates were incubated for 3 to 4 days at 37° C. in a CO₂ incubator. ZR-75-1 cells were harvested and pellets were stored frozen at −80° C. for further use. Pellets were dissolved in lysis buffer (Ripa-Buffer: 50 mM Tris-HCl pH 7.5; 150 mM NaCl; 1% Nonidet P-40; 0.5% sodiumdesoxycholate; 0.1% SDS; 1 mM EDTA) containing a protease inhibitor cocktail (PIC: protease inhibitor cocktail complete Mini, Roche). Cells were lysed for 10 min on ice and the lysate cleared by centrifugation. PIC was added and the protein content was determined by the DC protein assay (Bio-Rad Kit II) according to manufacturer's protocol. The Western blot was performed according to SOP-07-10. Briefly, cell lysates were diluted in reducing sample buffer and denatured for 10 min at 70° C. For SDS-PAGE, 30 μg protein per lane were loaded onto a 7.5% Tris-HCl ready gel (Bio-Rad). After blotting onto a nitrocellulose-membrane the membrane was blocked and the HER2 receptor was detected using a sheep-anti human ErbB2 antibody (Abcam). As secondary antibody a horse reddish peroxidase (HRP)-coupled rabbit anti-sheep antibodies (Abcam) was used. As a control for the loading of equal amounts of protein a second blot was done in parallel and incubated with a rabbit antibody against β-actin (Cell Signaling) and detected with a HRP-coupled a goat-anti-rabbit IgG H+L antibody (Jackson ImmunoResearch). The Western blot was developed using DAB Metal enhanced substrate kit (Thermo Scientific) according to manufacturer's protocol.

FIG. 4B shows an example of a Western blot analysis. The β-actin control shows that the same amount of cell lysate is loaded onto the gel. The HER2 receptor has a molecular weight of 185 kDa. The corresponding band is drastically reduced in the lysates of ZR-75-1 cells that were incubated with Fuc− trastuzumab or Herceptin® compared to the medium control or the isotype control antibody (h IgG1). Therefore, it was shown by Western blot and flow cytometry, that Fuc− trastuzumab down-modulates the expression of the HER2 receptor on ZR-75-1 cells in a comparable way as Fuc+ trastuzumab (Herceptin®).

### Example 13

### Inhibition of Proliferation of HER2 Expressing Tumor Cells

Binding of trastuzumab on the extracellular domain of HER2 results in the inhibition of proliferation of tumor cells (Brockhoff et al., 2007; Spiridon et al., 2002). In order to analyze this mechanism of action for Fuc− trastuzumab, proliferation of SK-BR-3 cells (human breast carcinoma cell line) was measured in an MTT assay with different concentrations (0.1-1 μg/ml) of Fuc− trastuzumab or Fuc+ trastuzumab (Herceptin®, Roche). MTT assay is a non-radioactive assay based on the cleavage of the soluble yellow tetrazolium salt MTT (3-[4,5-Dimethylthiazol-2-yl]-2,5-diphenyltetrazolium bromide; Thiazolyl Blue) by mitochondrial dehydrogenases of viable cells. This results in the formation of a purple formazan, which can be measured in an ELISA reader at 570 nm. The absorption signal is a direct measure of viable cells in the culture.

As a positive control, proliferation was completely inhibited by addition of taxol, and hlgG1 or medium alone served as negative controls. Briefly, SK-BR-3 cells were grown for 2 days in 96-well flat bottom plates. Fuc− trastuzumab, Herceptin® and control substances (hlgG1 and Taxol (20 nM)) were added and the plates were incubated for another 4-6 days at 37° C. in a humidified CO₂ incubator. Supernatant was completely removed and MTT was added. Cells were incubated for 2 hours with MTT at 37° C. in a humidified CO₂ incubator. The supernatant was removed and cells were lysed using HCl and 2-propanol containing lysis buffer for 1 h at room temperature in the dark. Absorption at 570 nm/630 nm was measured in a plate reader Infinite F200 (Tecan Austria GmbH).

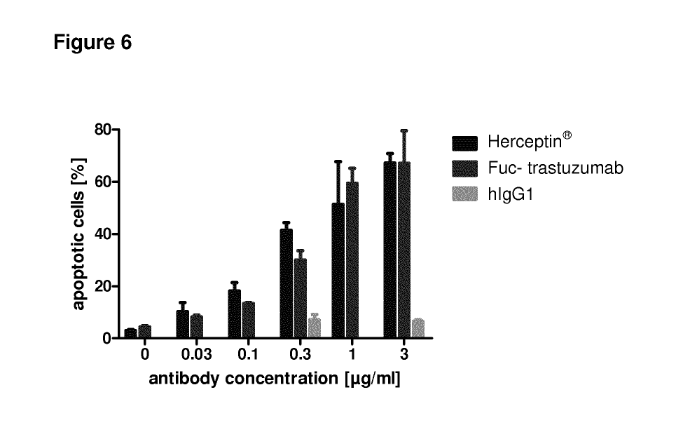

FIG. 5 shows the results of three independent experiments performed with Fuc− trastuzumab and Herceptin®. Proliferation after 4 days of incubation with the antibodies was calculated relative to the proliferation in the medium control. The positive control Taxol (20 nM) resulted in maximal proliferation inhibition (only 6% proliferation compared to the medium control; data not shown). Fuc− trastuzumab and Herceptin® induced a concentration-dependent inhibition of proliferation of SK-BR-3 cells. At an antibody concentration of 100 μg/ml, proliferation was reduced by more than 50%. Using Bonferroni posttests, there was no significant difference in the proliferation inhibition induced by Fuc− trastuzumab and Herceptin® to be observed. Compared to the human isotype control, there was a highly significant reduction in proliferation of SK-BR-3 cells observed at concentrations of 0.1 μg/ml and higher.

### Example 14

### Induction of Apoptosis of HER2 Expressing Tumor Cells

Induction of apoptosis is a further mechanism by which antibodies can mediate anti-tumor activity. While direct induction of apoptosis by monomeric antibodies is often ineffective (as seen for rituximab, Zhang et al., 2005) cross-linking of the antibody by anti-human immunoglobulin or protein G evokes this mechanism of action. In vivo, cross-linking of the antibody can be induced by Fc-receptor-bearing cells.

There are contradictory results published about the apoptotic activity of Herceptin® (Chakraborty et al., 2008; Brockhoff et al., 2007; Spiridon et al., 2002; De Lorenzo 2007)

In order to study this potential mode of action, the induction of apoptosis by Fuc− trastuzumab and Fuc+ trastuzumab (Herceptin®) was analyzed after cross-linking with protein G on the tumor cell line BT474. As a marker for induction of apoptosis, we analyzed the activation of caspase-3 using the BD PE Active Caspase-3 Apoptosis Kit. Caspase-3, a cystein protease, is a key protease that is activated during the early stages of apoptosis. It is synthesized as an inactive pro-enzyme of 32 kDa that is processed in cells undergoing apoptosis. The processed form consists of two subunits (17 kDa and 12 kDa) which associate to form the active caspase. Active caspase-3 proteolytically cleaves and activates other caspases as well as targets in the cytoplasm and in the nucleus, thereby promoting apoptosis. Activation of caspases is generally considered as the “point of no return” in apoptotic pathways. Using the BD PE Active Caspase-3 Apoptosis Kit, apoptotic cells are stained with an antibody specific for the active form of caspase-3 that does not recognize the inactive pro-enzyme form of caspase-3.

Briefly, tumor cell lines were cultured in medium containing 1% FCS for one day prior to the assay. Cells were seeded into 48 well plates incubated at 37° C. in a CO₂ incubator over night. Fuc− trastuzumab, Herceptin® or hlgG1 as a negative control at different concentrations and protein G at a final concentration of 2 μg/ml were added. The plates were incubated for 4 to 48 h at 37° C. in a CO₂ incubator.

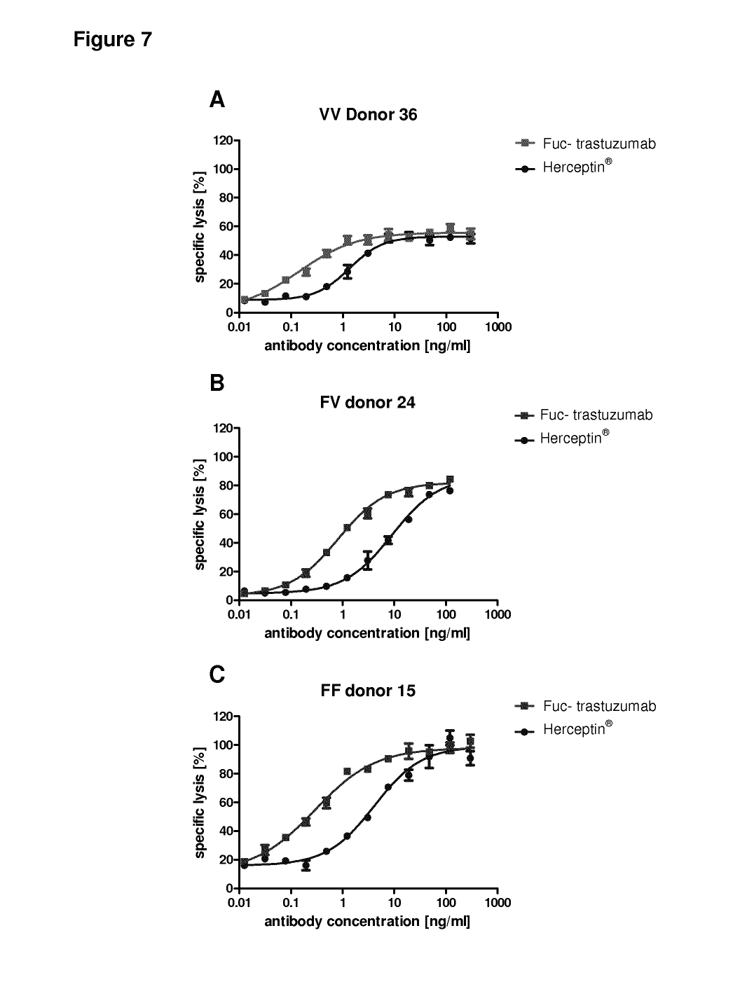

Cells (both adherent and non-adherent cells) were harvested, permeabilized, fixed and stained for active caspase-3 according to manufacturer's protocol. Active caspase-3-positive (apoptotic) cells were analyzed by flow cytometry at a BD FACS Canto II flow cytometer using BD FACSDiva™ Software. FIG. 6 shows the results of an active caspase-3 apoptosis assay using BT474 cells. After cross-linking by protein G, Fuc− trastuzumab induced strong concentration-dependent apoptosis in BT474 cells. Apoptosis induction was comparable between Fuc− trastuzumab and Herceptin® thereby confirming that Fab mediated tumor activities are retained.

### Example 15

### ADCC Activity of the Differently Fucosylated Antibody Variant

Reduction of fucose content within the glycosylation site in the antibody Fc domain is reported to lead to an increase of ADCC activity, the antibody-dependent cellular cytotoxicity resulting in a specific lysis of antigen positive tumor cells. This effect is caused by the higher affinity binding of the fucose-reduced antibody to the FcγRIIIa receptor on natural killer cells. Two allotypes of this receptor at amino acid position 158 (V158F) are known which have different affinities to human IgG1 antibodies. In general the V allele has a higher affinity to human IgG1 than the F allele receptor. Therefore, the ADCC activity of Fuc− trastuzumab in comparison to Fuc+ trastuzumab (Herceptin®) on donors with different FcγRIIIa receptors was analyzed: homozygeous V/V, homozygeous F/F and heterozygeous F/V donors were used for these studies. As the magnitude of ADCC activity is reported to be dependent on the expression level of the antigen on the cell surface, tumor cell lines expressing low or high HER2 levels were analyzed in the ADCC assay.

The assay was performed as an europium release assay. Briefly, HER2-positive target cell lines (SK-BR-3; MCF-7) were loaded with europium (Eu³⁺) by electroporation and incubated with thawed primary human peripheral blood mononuclear cells (PBMCs, effector cells, stored in liquid nitrogen) at an effector to target cell ratio (E:T ratio) of 50:1 in the presence of Fuc− trastuzumab, Herceptin® or human control antibodies at different concentrations for 5 hours. Europium release into the supernatant (indicating antibody mediated cell death) was quantified using a fluorescence plate reader Infinite F200 (Tecan Austria GmbH). Maximal release was achieved by incubation of target cells with triton-X-100 and spontaneous release was measured in samples containing only target cells alone. Specific cytotoxicity was calculated as:

% specific lysis=(experimental release−spontaneous release)/(maximal release−spontaneous release)×100.

The results of a number of experiments performed with both antibodies on the target cell line SK-BR-3 expressing high levels of HER2 (˜1×10⁶ binding sites per cell), and on the target cell line MCF-7 expressing low HER2 levels (˜3×10⁴ binding sites per cell) using effector cells from different donors of all three allotypes were analysed.

For the approximation of the magnitude of the ADCC enhancement of Fuc− trastuzumab compared to Herceptin®, concentration curves of Fuc− trastuzumab and Herceptin® were measured in parallel on the same plate for each donor. Curve fitting was performed for both antibodies separately using a four-parameter (4PL) logistic plot calculated by GraphPad Prism 5 software version 5.01. From the resulting curves, top and bottom lysis values and EC50 values were calculated. Furthermore, specific lysis values at certain antibody concentrations or the antibody concentration corresponding to certain specific lysis values were interpolated. Maximal specific lysis was calculated as the difference of top and bottom curve values.

Enhancement of ADCC Activity on SK-BR-3 Cells

Thirteen different donors (3 V/V donors, 5 F/V donors, 5 F/F donors) were analyzed for their ADCC activity on SK-BR-3 cells mediated by Fuc− trastuzumab or Herceptin®.

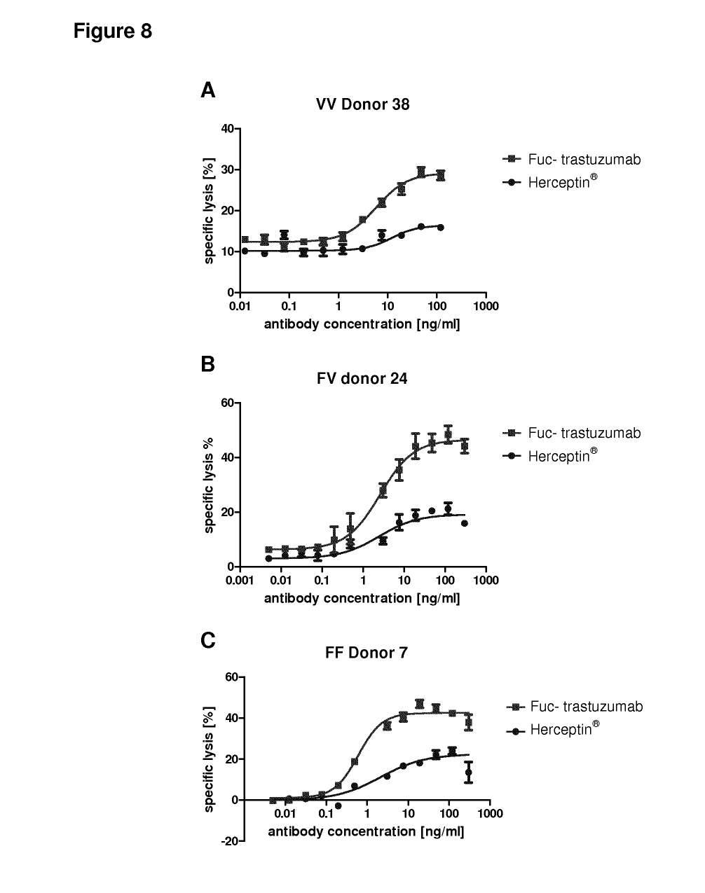

Fuc− trastuzumab mediates ADCC activities that are strongly enhanced compared to Herceptin® on all donor allotypes on the HER2 high level expression cell line SK-BR-3. In FIG. 7, representative examples of the concentration curves obtained with donors from the different allotypes are shown.

The maximal lysis achieved with Fuc− trastuzumab and Herceptin® was comparable for all donors and the curves showed comparable top and bottom values of specific lysis for Fuc− trastuzumab and Herceptin®. Therefore, the magnitude of increase of ADCC activity of Fuc− trastuzumab was estimated for all donors based on the comparison of the curves at other parameters: (i) the increase in specific lysis at a fixed antibody concentration, and (ii) the effective antibody concentration required for half maximal specific lysis of the both antibodies (EC50 values).

At a fixed antibody concentration of 0.5 ng/ml Fuc− trastuzumab shows a remarkable increase in specific lysis for all donor types (see FIG. 7). The mean Fuc− trastuzumab-mediated specific lysis from all 13 donors was 39% compared to 12% Herceptin®-mediated specific lysis. The mean difference of antibody-mediated specific lysis was highest on donors of the F/F allotype (60%) as compared to donors of the V/V or F/V allotype (28 and 26%, respectively). The mean increase factor was 6, indicating a 6 fold increase in ADCC activity of % tumor cells killed for Fuc− trastuzumab.

Furthermore, we compared the antibody concentration at which half maximal (50%) specific lysis is achieved (EC50 values) for Fuc− trastuzumab and Herceptin®. Higher efficacy of the antibody correlates with lower EC50 values. EC50 values were significantly different for both antibodies (p value 0.0009; two tailed paired student's t-test). Fuc− trastuzumab reaches half maximal specific lysis at 9-fold lower EC50 concentration values compared to Herceptin®. The improvement factor was higher for F/F and F/V donors.

In summary, the concentration curves obtained by Fuc− trastuzumab and Herceptin® in ADCC assay with 13 human donors of different allotypes were compared. While the curves showed comparable maximal lysis mediated by both antibodies, Fuc− trastuzumab showed an about 9-fold improvement of ADCC activities, as shown by the 9-fold reduction of the EC50 value and of the required concentration for the same specific lysis (for details see Table 4)

TABLE 4
Summary of analyses of ADCC assays of Fuc− trastuzumab
compared to Herceptin ® on SK-BR-3 cells.

| | Fuc− | Fuc+ | | Range of |
|---|---|---|---|---|
| | trastuzumab | trastuzumab | Factor | the factor |
| Maximal lysis [%] | 73.5 | 66.9 | 1 | 0.9-1.3 |
| EC50 value | 0.7 | 4.9 | 9 | 4-17 |
| Specific lysis at | 39.4 | 11.7 | 6 | 2-28 |
| 0.5 ng/ml [%] | | | | |
| Concentration required | 0.7 | 5.6 | 9 | 4-17 |
| for the same specific | | | | |
| lysis at EC50 of | | | | |
| Fuc− trastuzumab | | | | |
| [ng/ml] | | | | |
| Concentration required | 1.5 | 11.6 | 9 | 4-19 |
| for the same specific | | | | |
| lysis at 2x EC50 of | | | | |
| Fuc− trastuzumab | | | | |
| [ng/ml] | | | | |
| Concentration required | 0.4 | 3.0 | 9 | 6-17 |
| for the same specific | | | | |
| lysis at 0.5x EC50 of | | | | |
| Fuc− trastuzumab | | | | |
| [ng/ml] | | | | |

The mean values of the Fuc− trastuzumab and Herceptin ® values and the mean value of the individual factors of increase/improvement and the range of this factor among the 13 donors (5 FF, 3 VV, 5 FV) are given.

### Enhancement of ADCC Activity on MCF-7 Cells

Twelve different donors (3 V/V donors, 5 F/V donors, 4 F/F donors) were analyzed for their ADCC activity on MCF-7 cells mediated by Fuc− trastuzumab or Herceptin®.

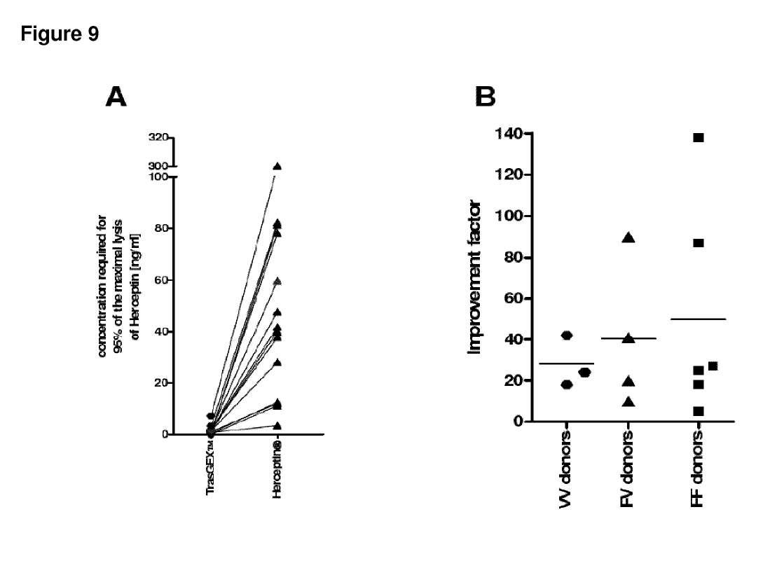

On MCF-7 cells, Fuc− trastuzumab mediates ADCC activities that are strongly enhanced compared to Herceptin® on all donor allotypes. In FIG. 8 representative examples of the concentration curves obtained with donors from the different allotypes are shown.

MCF-7 cells express about 30 times less HER2 antigen on the cell surface than SK-BR-3 cells. As the ADCC activity correlates with antigen density on the cell surface, a lower maximal lysis was expected for MCF-7 cells as compared to SK-BR-3 cells. Indeed, the mean maximal specific lysis of Fuc− trastuzumab was only 40% compared to 74% on SK-BR-3 cells. This is consistent with a report by Suzuki and coworkers (2007) showing higher ADCC activities of Fuc+ trastuzumab on SK-BR-3 cells as compared to MCF-7 cells. Strikingly, the maximal lysis of MCF-7 cells obtained with Fuc− trastuzumab was drastically enhanced compared to Herceptin® (p value<0.0001). While the maximal lysis mediated by Fuc− trastuzumab was between 17% and 72%, Herceptin® mediated maximal lysis was much lower ranging from 6 to 29%. The mean maximal lysis mediated by Fuc− trastuzumab was increased by a factor of 3 (2× to 5×).

Due to the differences in the maximal specific lysis obtained by Fuc− trastuzumab and Fuc+ trastuzumab a comparison of the EC50 values of the antibodies is not informative, as the EC50 value could be the same despite much higher specific lysis of Fuc− trastuzumab. Therefore, the differences of the binding curves of Fuc− trastuzumab and Fuc+ trastuzumab was analyzed by comparing (i) the specific lysis achieved at 10 ng/ml and (ii) the concentrations required for the same specific lysis.

The specific lysis that was mediated by Fuc− trastuzumab and Fuc+ trastuzumab at an antibody concentration of 10 ng/ml shows a significant difference between both antibodies (p value<0.0001). A mean increase in specific lysis of 18% was observed, corresponding to a 3 times higher specific lysis at an antibody concentration of 10 ng/ml obtained with Fuc− trastuzumab in comparison to Fuc+ trastuzumab.

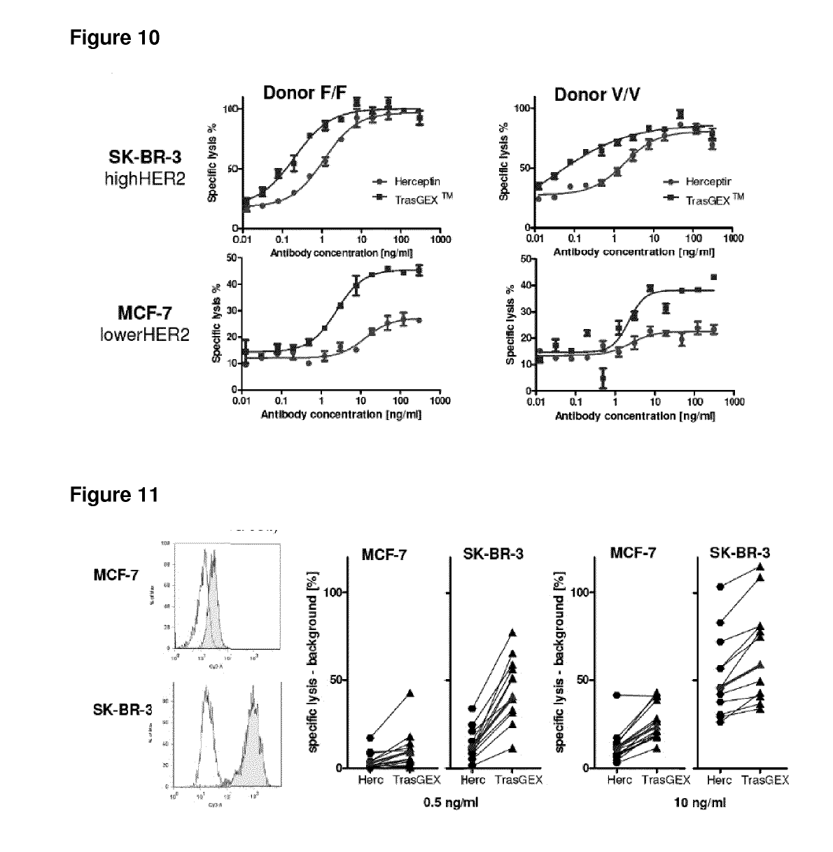

There was a significant difference between the required antibody concentration of Fuc− trastuzumab and Fuc+ trastuzumab that is required for 95% of the maximal specific lysis (p value 0.03). The required antibody concentration in order to achieve the same specific lysis at this point was reduced by factor 5 to factor 138 (improvement factor) among the different donors (see FIG. 9). The reduction in required antibody concentrations is highest for FF donors (improvement factors: FF donors 55, VV donors 28, FV donors 40).

At the HER2 low level expressing MCF-7 cells, comparison of the ADCC activity of Fuc− trastuzumab and Fuc+ trastuzumab at three different points of the concentration curve, showed a drastic increase of ADCC activity for the fucose-reduced trastuzumab antibody. The maximally achieved specific lysis (corresponding to the percentage of target cells the antibody is capable to kill) and the specific lysis at 10 ng/ml were increased by up to 5 fold. For the same specific lysis, up to 138 times lower antibody concentrations of Fuc− trastuzumab were required in comparison to Fuc+ trastuzumab (see Table 5).

TABLE 5
Summary of analyses of ADCC assays of Fuc− trastuzumab
compared to Fuc+ trastuzumab on MCF-7 cells.

| | Fuc− | Fuc+ | | Range of |
|---|---|---|---|---|
| | trastuzumab | trastuzumab | Factor | the factor |
| Maximal lysis [%] | 40 | 16 | 3 | 2-5 |
| Specific lysis at | 30 | 11 | 3 | 2-5 |
| 10 ng/ml [%] | | | | |
| Concentration required | 2 | 58 | 43 | 5-138 |
| for the same specific | | | | |
| lysis at 95% of | | | | |
| maximal lysis of | | | | |
| Herceptin ® [ng/ml] | | | | |

The mean values of the Fuc− trastuzumab and Fuc+ trastuzumab values and the mean value of the individual factors of increase/improvement and the range of this factor among the 12 donors (5 FF, 3 VV, 4 FV) are given.

### Further Results

A characterization of cell lines according to their HER2 status is given in subsequent table 6:

TABLE 6
HER2 status of different cell lines

| | HER2 | HER2 | | | FACS |
|---|---|---|---|---|---|
| | (IHC, | (WB, % of | | FACS | (MFI at, |
| | Hercep | BT474 | | (% pos. | 1 μg/ml, % |
| | Test) | HER2) | FISH | cells) | of BT474) |
| BT474 | 3+ | 100 | Ampl | 99 | 100 |
| SK-BR-3 | 3+ | 103 | Ampl | 98 | 83 |
| NCI-N87 | 2+ | 241 | Ampl | 93 | 83 |
| SKOV-3 | 3+ | 176 | Ampl | 99 | 12 |
| ZR-75-1 | 2+ | 21 | Not ampl | 50 | 7 |
| PANC-1 | 1+ | | | 22 | 4 |
| MCF-7 | 0-1+ | 1.03 | Not ampl | 31 | 4 |

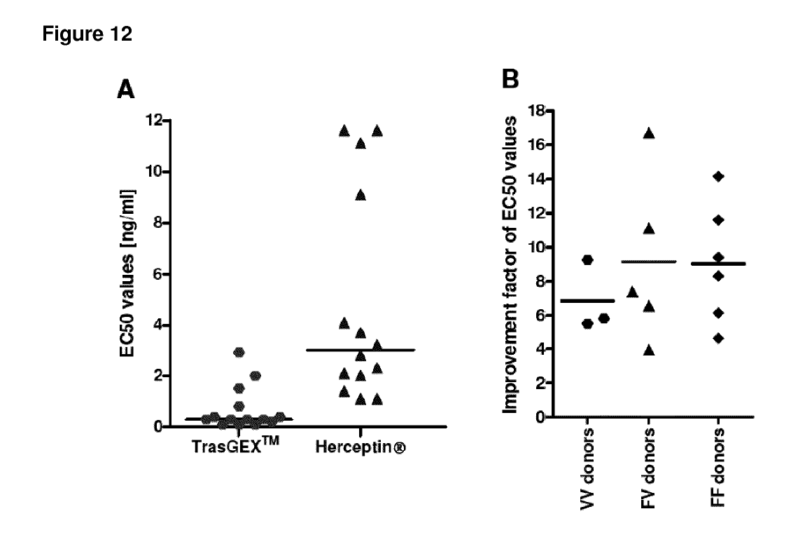

Further results in experiments similar to the ones described above are also shown in FIGS. 10 to 12. FIG. 10 shows that the glycooptimization of the Fuc− trastuzumab (low fucose, increased bisGlcNAc) leads to highly improved ADCC activity for the treatment of all patient subgroups, especially those with lower HER2 expressing tumors. The results shown in FIG. 11 confirm that increased ADCC responses are observed with all PBMC donors. 10 to 140 less antibody concentration is needed for the same ADCC response. The Fuc− trastuzumab showed an enhanced ADCC response in high HER2 cells (SK-BR-3) as well as in low HER2 cells (MCF-7). FIG. 12 shows the EC50 values obtained from ADCC assays with 14 donors of different allotypes for Fuc− trastuzumab as described in example 1 (also referred to as TrasGEX™) using high HER2 SK-BR-3 cells. FIG. 12A shows the EC50 values of the 14 individual donors. Each symbol represents an individual donor. Median levels are given as lines. FIG. 12B shows the improvement factor of the donors (EC50 Herceptin®/EC50 Fuc− trastuzumab). Each symbol represents an individual donor. Mean values are given as lines.

### Summary of the Analyses of ADCC Assays of Fuc− Trastuzumab Compared to Fuc+ Trastuzumab

Comparison of the ADCC activity of Fuc− trastuzumab and Fuc+ trastuzumab (Herceptin®) shows a drastic increase of ADCC activity for the fucose-reduced trastuzumab antibody for tumor cells expressing high as well as low HER2 levels. ADCC increase mediated by Fuc− trastuzumab is especially prominent on tumor cells expressing low HER2 levels.

As is demonstrated by the results, the maximum Fuc− trastuzumab mediated ADCC increase for low HER2 expressing tumors (MCF-7) is up to ˜140 fold, with 43-fold in the mean, and for high HER2 expressing tumors (SK-BR-3) up to ˜30 fold for maximum increase with 9-fold in the mean. Even the maximal % tumor cell lysis is strongly increased with up to 5 fold (mean 3-fold) for low HER2 expressing tumors when using Fuc− trastuzumab. The high effectiveness on low HER2 expressing tumors provides important therapeutic options for the reduced fucose anti-HER2 antibodies as a treatment of HER2 positive cancers which only show a low or moderate amount of HER2 overexpression becomes possible. As described above, a therapeutic effect was even seen in patients showing a HER2 overexpression of only 1+(HER2 1+) as determined by IHC.

Thus, reduced fucose anti-HER2 antibodies according to the present invention show a high increase in the ADCC activity which is a key mode of tumor action. Furthermore, the effects achieved due to the optimized glycosylation allow to broaden the suitable patient spectra to patients so far not benefitting from the corresponding high fucose anti-HER2 antibodies. As is shown herein, a therapeutic effect is seen for all FcγRIIIa allotypes instead of less than 20% for a high fucose anti-HER2 antibody such as Herceptin®. Furthermore, also patients with lower HER2 expression can benefit from the described treatments. This is an important effect, in particular considering the novel therapeutic effects seen on metastases, in particular ulcerating skin metastases and visceral metastases such as lung and liver metastases.

The increased ADCC activity of Fuc− trastuzumab which has been demonstrated by these in vitro experiments is based especially on the low fucose content (less than 10%) and furthermore, is supported by the enhanced amount of bisecting GlcNAc (more than 10%) of the glycosylation of the Fc fragment.

### Example 16

### In Vivo Pharmacology of the Differently Fucosylated Antibody Variant

Several in vivo studies were performed in mice and cynomolgus monkeys to investigate the pharmacological effects of Fuc− trastuzumab, some of them were performed in comparison to Fuc+ trastuzumab (Herceptin®).

Tissue cross-reactivity studies with Fuc+ trastuzumab showed that the antibody reacted only with human and cynomolgus monkey tissue but not with rodent tissues or tissues of any other animal species. As Fuc− trastuzumab shows the same antigen binding specificities, affinities and mode of action as Fuc+ trastuzumab it is expected that its tissue reactivity is identical to Herceptin®. Therefore, rodent studies using xenograft models of human tumor cells are considered as important efficacy studies.

Unlike rodents the cynomolgus monkey was considered as an appropriate species for safety and toxicity testing of Fuc+ trastuzumab and is therefore also relevant for the toxicity testing of Fuc− trastuzumab.

### Antitumor Activity in Animal Models

A pharmacodynamic study in nude mice analysing the efficacy of Fuc− trastuzumab in different tumor models was performed and also compared to Fuc+ trastuzumab. Athymic nude mice xenografted with HER2 positive tumor cells from a human cell line BT474 or from patient derived carcinomas were used.

In the study 1×10⁷ tumor cells were subcutaneously (s.c.) implanted into nude mice (N=8 per group) and allowed to grow until tumors reached palpable size, which was reached approximately 7-13 days post implantation. The target tumor size was ˜0.1 cm³. At this time point antibody treatment was started. Tumor size was measured twice weekly with a calliper-like instrument. Individual tumor volumes were calculated (V=(length+width²)/2) and related to the values at the first day of treatment (relative tumor volume). The therapeutic effect was determined in terms of primary tumor growth inhibition.

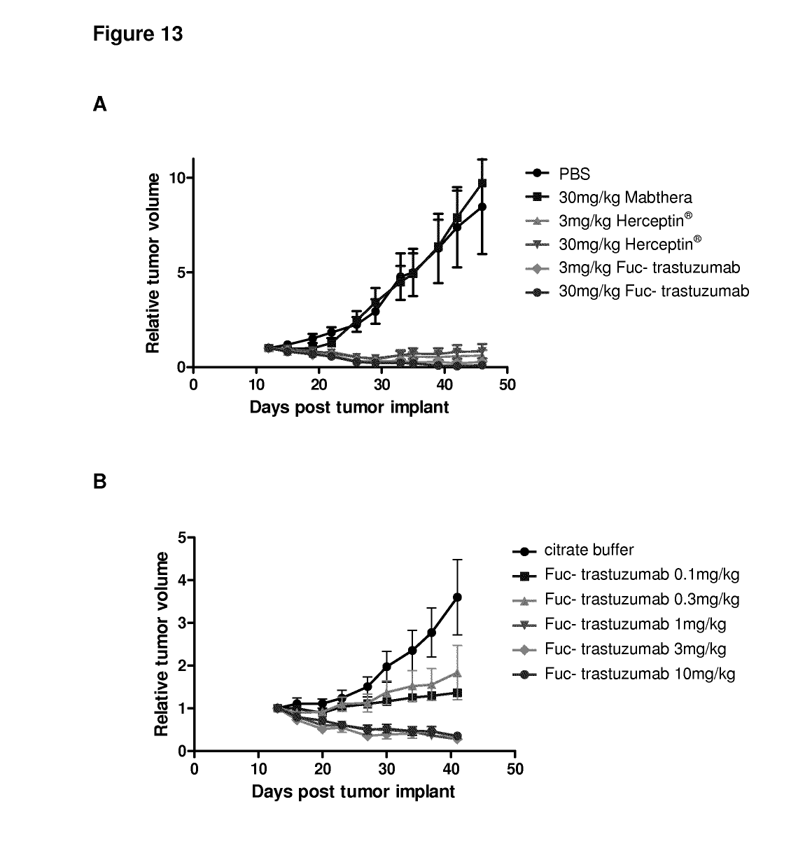

Fuc− trastuzumab and Fuc+ trastuzumab (N=8f/group) were administered intravenously twice weekly for 4 weeks at dose levels of 3 mg/kg and 30 mg/kg. The application volume was 10 μl/g body weight for both antibody formulations. Adjustment of the concentration in the injection solution was done by dilution with PBS. For the set of experiments a body weight based dosing was selected to enhance the dosing accuracy and comparability during the treatment period (Fichtner et al. 2008, Steiner et al. 2007). Mabthera® (Roche) served as an irrelevant antibody control and was administered only at a dose level of 30 mg/kg. Xenografted mice were treated at the indicated dosage level when tumors reached palpable size. Each symbol represents the mean value and SEM of a group of 8 animals. The mean relative tumor volumes of the treated animals are shown in FIG. 13A.

Both antibodies, the Fuc− trastuzumab as well as the Fuc+ trastuzumab inhibit strongly the BT474 tumor growth compared to PBS treated animals (p<0.001). No significant difference between the dose levels was observed. Fuc− trastuzumab caused tumor remissions in 8 of 8 tumors, Herceptin® caused tumor remissions in 7 of 8 tumors. No significant difference between the relative tumor volume and the number of tumor remissions in the Fuc− trastuzumab treated group and the Fuc+ trastuzumab treated group was found in the dose groups. Therefore, this experiment verifies that the Fuc− trastuzumab shows a strong dose dependent anti-tumor activity. A comparable efficacy of Fuc− trastuzumab and Fuc+ trastuzumab was expected in this model, as mice are not sensitive for the performed glycoptimization, i.e. the reduction in fucose and the increase in bisGlcNAc. The improvements and new therapeutic options of the reduced fucose anti-HER2 antibodies according to the present invention are in particular demonstrated by the clinical data shown herein. No significant changes in the body weight of the animals were observed indicating that no toxicity occurred.

A second study was performed to investigate the dose dependency of the antitumor effect of Fuc− trastuzumab. Five different dose levels ranging from 0.1 mg/kg to 10 mg/kg Fuc− trastuzumab were investigated. Fuc− trastuzumab (N=8) was intravenously administered twice weekly for 4 weeks. The mean relative tumor volume of the animals is shown in FIG. 13B. Fuc− trastuzumab inhibited strongly and dose-dependently the BT474 tumor growth compared to vehicle treated animals (p<0.001).

No significant changes in the body weight of the animals were observed indicating that no toxicity occurred.

### Example 17

### Patient Derived Tumor Model

The antitumor activity of Fuc− trastuzumab was studied in immune deficient nude mice bearing human patient derived carcinoma xenografts of gastric origin. Xenografts of patient derived tumor cells are supposed to be more similar to the original tissue than tumor cell lines and therefore considered to be of higher clinical relevance. Tumor model was selected according to its positive HER2 expression status which has been evaluated immunohistochemically. The tumor of gastric carcinoma origin was shown to express moderate levels of HER2 by immunohistochemistry.

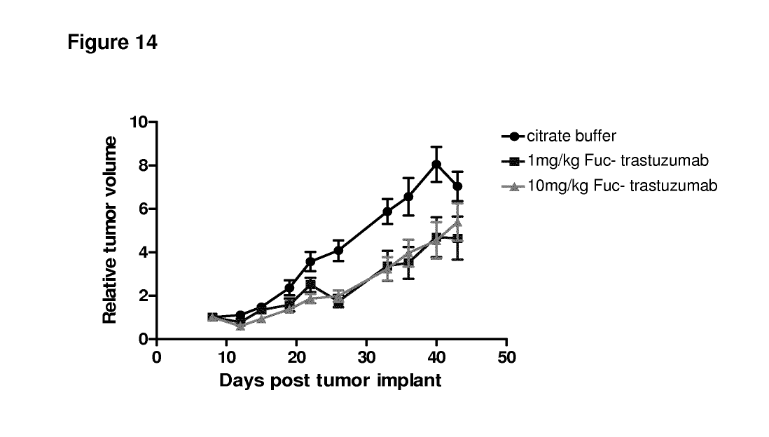

In the gastric model, Fuc− trastuzumab (N=8 m/group) was administered i.v. twice weekly for 4 weeks at dose levels of 1 mg/kg and 10 mg/kg. Adjustment of the concentration in the injection solution was done by dilution with formulation buffer. The application volume was kept constant at 10 μl/g body weight. The mean relative tumor volume of the animals is shown in FIG. 14.

Fuc− trastuzumab inhibited the tumor growth significantly (p<0.001), thereby confirming that the reduced fucose anti-HER2 antibodies according to the present invention are suitable for treatment of HER2 positive tumors which also express moderate levels of HER2. No major difference in efficacy was observed between the two tested dose levels, again confirming that the reduced fucose anti-HER2 antibodies according to the present invention is already highly effective at small dosages. No animal died prematurely. No significant changes in the body weight of the animals were observed indicating that no major toxicity occurred.

### Example 18

### Pharmacokinetics

The PK/TK profile of Fuc− trastuzumab was investigated in a single dose PK study to evaluate the serum half-life of Fuc− trastuzumab in nude mice in comparison to Herceptin®.

Furthermore, the PK/TK profile of Fuc− trastuzumab was characterized in a single dose cynomolgus monkey study comparing the PK profile of Fuc− trastuzumab and Herceptin®. Plasma samples collected in this study were analyzed by an ELISA method.

### Serum Half-Life in Nude Mice

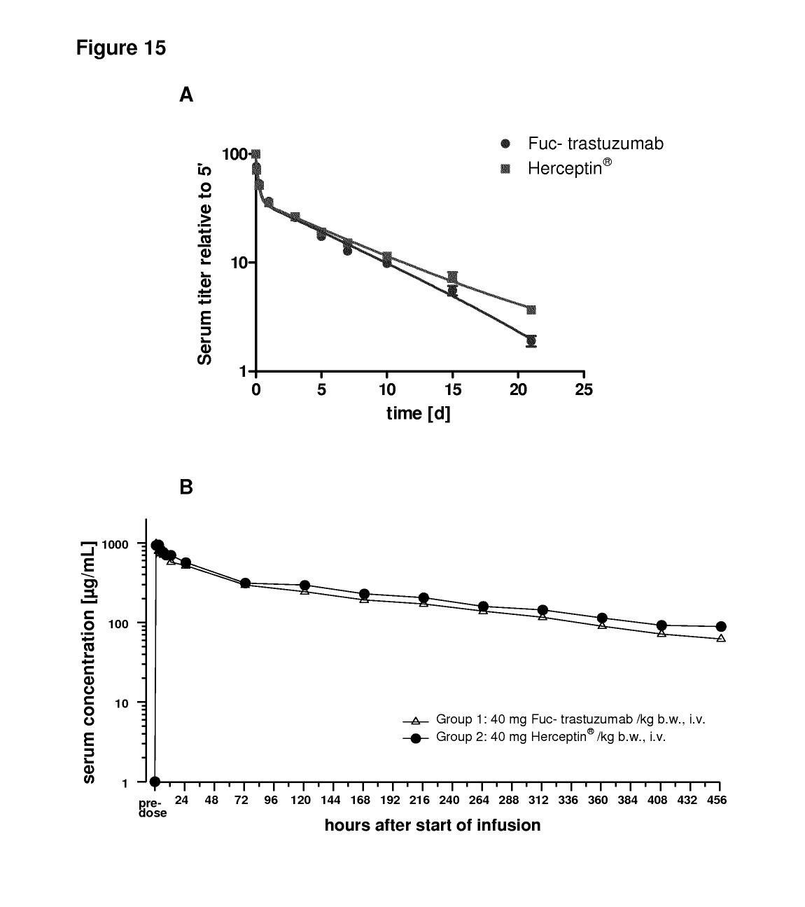

The pharmacokinetic behavior of Fuc− trastuzumab and Herceptin® was studied in nude mice following a single intravenous (i.v.) bolus administration of 30 mg/kg body weight in an application volume of 10 ml/kg body weight (N=3 f/group). Dose levels of 1 mg/kg up to 100 mg/kg are considered to be within the efficacious range (Fujimoto-Ouchi et al., 2007; Baselga et al., 1998; Pietras et al., 1998) and were also used in single dose pharmacokinetic studies with Herceptin® (EMEA, EPAR for Herceptin®). Blood samples were taken predose (−1d), at 5 min, 1, 6, 24 hours and 3, 5, 7, 10, 15, 21 d post dosing. The antibody serum levels were determined by a commercial titer ELISA assay. The calibration range of the assay was 1 up to 1000 ng antibody per ml serum. The results are shown in FIG. 15A.

The concentration of the injection solution of Fuc− trastuzumab and Herceptin® was adjusted by dilution with PBS.

Fuc− trastuzumab as well as Herceptin® exhibited a two phase exponential decay with an initial half-life of 3.5 h and 3.0 h, respectively. The terminal half-life of Fuc− trastuzumab is 5.4 d (equals 130 h) and 5.5 d (equals 132 h) for Herceptin®. The maximum plasma concentration was 801±151 μg/ml for Fuc− trastuzumab and 868±84 μg/ml for Herceptin®. These differences in the plasma concentration and the half life are not statistically significant, thus, the pharmacokinetic behavior of both antibodies is considered to be similar. These results are in good agreement with published data (Palm et al., 2003; t_{1/2}=110 h).

Pharmacokinetic Study of Fuc− Trastuzumab in Comparison to Herceptin after Single Intravenous 1-h Infusion to Cynomolgus Monkeys

The aim of this study was to evaluate the pharmacokinetics of Fuc− trastuzumab in comparison to the reference product Herceptin® after a single 1 hour i.v. infusion to cynomolgus monkeys followed by a 20-day observation period.

2 groups, each comprising 3 male cynomolgus monkeys were treated by a single 1 hour i.v. infusion with either the Fuc− trastuzumab or Fuc+ trastuzumab (Herceptin®) at a dose of 40 mg/kg body weight (bw). The dose was selected referring to pharmacokinetic and toxicological studies in rhesus and cynomolgus monkeys which used doses of 1-47 mg/kg Herceptin® (EMEA, EPAR for Herceptin®).

Animals were observed individually before and after dosing at each time of dosing for any signs of behavioral changes, reaction to treatment or illness. Cage side observations included skin/fur, eyes, mucous membranes, respiratory and circulatory systems, somatomotor activity and behavior patterns. Special attention was paid to the local tolerance of the test or reference item at the infusion site. The body weight of each monkey was recorded predose, at study initiation and thereafter in weekly intervals always on the same day of the week at the same time of the day.

Blood sampling was performed prior to infusion, immediately (within 2 minutes) after the end of the 1 hour infusion and 2, 4, 6, 8, 12 and 24 hours after the end of the infusion. Furthermore on test days 4, 6, 8, 10, 12, 14, 16, 18 and 20 after end of the infusion blood samples were collected. Standard toxicokinetic parameters were assessed.

None of the animals died prematurely during the course of the study or showed clinical signs of systemic toxicity. No test item-related influence or local intolerance was noted. Cₘₐₓ levels were observed immediately (within 2 minutes) after the end of the 1 hour infusion for both, Fuc− trastuzumab and Herceptin®, and were found to be in the same range. The mean terminal serum elimination half-life of Fuc− trastuzumab was 170 hours while the mean terminal serum elimination half-life of Herceptin® was 195 hours, each on test day 1. There were no statistically significant differences (at p≦0.01) between the toxicokinetic parameters of Fuc− trastuzumab compared to Herceptin® (FIG. 15B). Therefore, the reduced fucose anti-HER2 antibodies according to the present invention show a similar pharmacokinetic behavior as the corresponding high fucose anti-HER2 antibody. This confirms, that the improved and novel therapeutic effects that are seen with reduced fucose anti-HER2 antibodies according to the present invention are indeed attributable to the tumor activity that is changed due to the changed glycosylation and is not attributable to a change in the pharmacokinetic behavior.

The mean toxicokinetic parameters in monkey serum are given in Table 7.

TABLE 7
Toxicokinetic Parameters calculated
after a single antibody infusion.

| | | Cₘₐₓ^{#} | tₘₐₓ^{#,##} | t_{1/2} | AUC_{0-t last} | AUC_{0-∞} |
|---|---|---|---|---|---|---|
| Dosage | | [μg/mL] | [h] | [h] | [μg*h/mL] | [μg*h/mL] |
| 40 mg Fuc- | Mean | 970.6 | 1.67 | 170.27 | 92877.5 | 108755.3 |
| trastuzumab/ | SD | 192.9 | 1.15 | 33.89 | 13427.9 | 20822.2 |
| kg | | | | | | |
| 40 mg | Mean | 958.2 | 2.33 | 194.97 | 107565.2 | 131068.3 |
| Herceptin ®/ | SD | 72.0 | 1.15 | 8.98 | 3184.5 | 4254.7 |
| kg | | | | | | |

SD standard deviation;
^{#}Values obtained from serum analysis of Fuc- trastuzumab and Herceptin ®, all other values calculated by toxicokinetic analysis;
^{##}time after end of infusion

Repeated dose toxicity studies were performed in cynomolgus monkeys. In the dose range finding study, the following dose levels were tested: 5, 20, and 40 mg/kg bw/day Fuc− trastuzumab. The dosing schedule was twice weekly, for two weeks and five administrations in total. The administration road was one hour—iv infusions. The standard toxicological parameters were studied and no treatment-related effects were observed.

In a pivotal four week repeated dose study, the following dose levels were tested: 40, 20 and 5 mg/kg Fuc− trastuzumab. The dosing schedules were weekly, for four weeks, and five administrations in total. The administration road was one hour—iv infusions. Standard toxicological parameters including immunogenicity and safety pharmacological parameters were studied. No adverse effects and no-observed-adverse-effect-level above 40 mg/kg b.w./dose was found. Furthermore, tissue cross-reactivity in human and cynomolgus monkey tissue was performed and the tissue cross-reactivity was found to be comparable to the Fuc+ trastuzumab (Herceptin®).

1. A method for treating a human patient with a HER2 positive metastasizing cancer, comprising administering an anti-HER2 antibody having an amount of fucose in the CH2 domain of 50% or less (reduced fucose anti-HER2 antibody) to the patient.

2. The method according to claim 1, wherein the reduced fucose anti-HER2 antibody has the following glycosylation characteristics in the CH2 domain:
(i) a relative amount of carbohydrate chains carrying fucose of 20% or less;
(ii) a relative amount of carbohydrate chains carrying bisecting GlcNAc of at least 8%;
(iii) a relative amount of carbohydrate chains carrying at least one galactose unit of at least 65%; and
(iv) a relative amount of carbohydrate chains carrying at least two galactose units of at least 15%.

3. The method according to claim 2, wherein prior to the treatment with the reduced fucose anti-HER2 antibody said patient has been treated with at least one anti-HER2 antibody having an amount of fucose in the CH2 domain of 60% or more (high fucose anti-HER2 antibody).

4. The method according to claim 1, wherein the metastasizing cancer includes one or more of skin metastases, visceral metastases, and lymph node metastases.

5. The method according to claim 4, wherein the patient has one or more visceral metastases, selected from the group consisting of lung and liver metastases.

6. The method according to claim 1, wherein the metastasizing cancer is breast cancer.

7. The method according to claim 1 wherein the metastazing cancer is selected from the group consisting of colon cancer, salviary gland cancer, lung cancer, and bronchial cancer.

8. The method according to claim 1, wherein the metastasizing cancer has a HER2 overexpression of level 2+ or lower.

9. The method according to claim 1, for the treatment of skin lesions or lymph node lesions caused by a metastasis.

10. The method according to claim 1, wherein prior to the treatment with the reduced fucose anti-HER2 antibody said patient has been treated with
a) at least one chemotherapeutic agent; and/or
b) at least one anti-HER2 antibody having an amount of fucose in the CH2 domain of 60% or more (high fucose anti-HER2 antibody), or at least one anti-HER2 antibody which is not glycosylated;
wherein the preceding treatments a) and b) occurred in any order sequentially or concurrently.

11. The method according to claim 10, wherein prior to the treatment with the reduced fucose anti-HER2 antibody the patient has been treated with at least five different anti-cancer agents.

12. The method according to claim 10, wherein the HER2 positive cancer is resistant to or has progressed after treatment with at least one chemotherapeutic agent and/or is resistant to or has progressed after treatment with high fucose trastuzumab and/or high fucose pertuzumab.

13. The method according to claim 1, wherein the reduced fucose anti-HER2 antibody is repeatedly administered to the patient and wherein a therapeutic effect is obtained at least after the second administration of the reduced fucose anti-HER2 antibody.

14. The method according to claim 13, wherein the therapeutic effect includes a reduction of skin lesions, a reduction of mediastinal adenopathies and/or a reduction of visceral metastases.

15. The method according to claim 1, wherein the anti-HER2 antibody has an amount of fucose in the CH2 domain of 20% or less.

16. The method according to claim 1, wherein the anti-HER2 antibody has the following glycosylation characteristics in the CH2 domain:
(i) an amount of bisecting GlcNAc of at least 8%;
(ii) an amount of galactose of at least 65%;
(iii) no detectable NeuGc;
(iv) no detectable Galα1,3-Gal; and
(v) detectable α2,6-coupled NeuAc.

17. The method according to claim 1, wherein the antibody has one or more characteristics selected from the group consisting of
(i) a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3;
(ii) comprises a heavy chain variable region comprising the amino acid sequence of SEQ ID NO: 7 or an amino acid sequence which is at least 80% identical thereto;
(iii) a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6;
(iv) comprises a light chain variable region comprising the amino acid sequence of SEQ ID NO: 8 or an amino acid sequence which is at least 80% identical thereto;
(v) shows cross-specificity with the antibody trastuzumab; and
(vi) is an IgG antibody.

18. The method according to claim 1, wherein the treatment with the reduced fucose anti-HER2 antibody is a monotherapy; or wherein the treatment with the reduced fucose anti-HER2 antibody is a combination therapy selected from the group consisting of
(i) at least one chemotherapeutic agent;
(ii) at least one further therapeutic antibody which is different from the reduced fucose anti-HER2 antibody;
(iii) cancer surgery; and
(iv) radiotherapy.

19. The method according to claim 1, wherein the administration of the reduced fucose anti-HER2 antibody is in an amount of 1 to 15 mg/kg body weight of the patient every first, second, third or fourth week.

20. The method according to claim 1, wherein the reduced fucose anti-HER2 antibody is for treatment of patients irrespective of their FcγRIIIa allotype.

21. The method of claim 10, wherein prior to the treatment with the reduced fucose anti-HER2 antibody said patient has been further treated with
c) radiotherapy; and/or
d) at least one further therapeutic antibody.
wherein the treatments a), b), c) and d) occurred in any order sequentially or concurrently.
